SEQUENCE LISTING (87 sequences)

SEQ ID NO: 1 - DNA, 1353 bp, synthetic construct
  misc_feature 1..1353: note = Synthetic: Ab1 heavy chain DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatctgtctactgccagctcattggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 451 aa, synthetic construct
  REGION 1..451: note = Synthetic: Ab1 heavy chain peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 363 bp, synthetic construct
  misc_feature 1..363: note = Synthetic: Ab1 VH DNA
  source 1..363: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatctgtctactgccagctcattggactattggggccagggcaccctcgtaacggtgtcatct

SEQ ID NO: 4 - protein, 121 aa, synthetic construct [sequence omitted]

SEQ ID NO: 5 - DNA, 642 bp, synthetic construct
  misc_feature 1..642: note = Synthetic: Ab1 light chain DNA
  source 1..642: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagatataatcgggcgccatataccttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - protein, 214 aa, synthetic construct
  REGION 1..214: note = Synthetic: Ab1 light chain peptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 321 bp, synthetic construct
  misc_feature 1..321: note = Synthetic: Ab1 VL DNA
  source 1..321: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagatataatcgggcgccatataccttcggccagggtacgaaagtcgaaatcaaa

SEQ ID NO: 8 - protein, 107 aa, synthetic construct [sequence omitted]

SEQ ID NO: 9 - DNA, 1353 bp, synthetic construct
  misc_feature 1..1353: note = Synthetic: Ab1 heavy chain ( holes ) DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatctgtctactgccagctcattggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgtaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 451 aa, synthetic construct
  REGION 1..451: note = Synthetic: Ab1 heavy chain ( holes ) peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - DNA, 681 bp, synthetic construct
  misc_feature 1..681: note = Synthetic: heavy chain 2 DNA
  source 1..681: mol_type = other DNA
gacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgtcgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 227 aa, synthetic construct
  REGION 1..227: note = Synthetic: heavy chain 2 peptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1288 bp, synthetic construct
  misc_feature 1..1288: note = Synthetic: 2631/VH1 heavy chain DNA
  source 1..1288: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 451 aa, synthetic construct
  REGION 1..451: note = Synthetic: 2631/VH1 heavy chain peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYHSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 363 bp, synthetic construct
  misc_feature 1..363: note = Synthetic: 2631/VH1 VH DNA
  source 1..363: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatcattctactgccagctcattggactattggggccagggcaccctcgtaacggtgtcatct

SEQ ID NO: 16 - protein, 121 aa, synthetic construct
  REGION 1..121: note = Synthetic: 2631/VH1 VH peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYHSTASSLDYWGQGTLVTVSS

SEQ ID NO: 17 - DNA, 1353 bp, synthetic construct
  misc_feature 1..1353: note = Synthetic: 2631/VH1 heavy chain ( holes ) DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatcattctactgccagctcattggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgtaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 451 aa, synthetic construct
  REGION 1..451: note = Synthetic: 2631/VH1 heavy chain ( holes ) peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYHSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - DNA, 1353 bp, synthetic construct
  misc_feature 1..1353: note = Synthetic: 2632/VH2 heavy chain DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatctgtctactgcccatcatttggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 451 aa, synthetic construct
  REGION 1..451: note = Synthetic: 2632/VH2 heavy chain peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTAHHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 363 bp, synthetic construct
  misc_feature 1..363: note = Synthetic: 2632/VH2, VH DNA
  source 1..363: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatctgtctactgcccatcatttggactattggggccagggcaccctcgtaacggtgtcatct

SEQ ID NO: 22 - protein, 121 aa, synthetic construct
  REGION 1..121: note = Synthetic: 2632/VH2, VH peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTAHHLDYWGQGTLVTVSS

SEQ ID NO: 23 - DNA, 1353 bp, synthetic construct
  misc_feature 1..1353: note = Synthetic: 2632/VH2 heavy chain ( holes ) DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggtttcctatctgtctactgcccatcatttggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgtaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 451 aa, synthetic construct
  REGION 1..451: note = Synthetic: 2632/VH2 heavy chain ( holes ) peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTAHHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - DNA, 1353 bp, synthetic construct
  misc_feature 1..1353: note = Synthetic: 2633/VH3 heavy chain DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggttcattatcattctactgccagctcattggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - protein, 451 aa, synthetic construct
  REGION 1..451: note = Synthetic: 2633/VH3 heavy chain peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVHYHSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 363 bp, synthetic construct
  misc_feature 1..363: note = Synthetic: 2633/VH3, VH DNA
  source 1..363: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggttcattatcattctactgccagctcattggactattggggccagggcaccctcgtaacggtgtcatct

SEQ ID NO: 28 - protein, 121 aa, synthetic construct
  REGION 1..121: note = Synthetic: 2633/VH3, VH peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVHYHSTASSLDYWGQGTLVTVSS

SEQ ID NO: 29 - DNA, 1353 bp, synthetic construct
  misc_feature 1..1353: note = Synthetic: 2633/VH3 heavy chain ( holes ) DNA
  source 1..1353: mol_type = other DNA
gaggtccagcttgtagaaagtggaggcggtttggtgcaacccggacgcagtctgcgactgtcttgtgctgcaagtgggttcacattcgacgattatgcaatgcactgggtgcggcaggctcctggtaaggggctggagtgggtctcagcaattacatggaactccgggcatattgattacgctgattccgtagagggtaggttcacgatctctcgggataatgcgaaaaattcactctatctgcaaatgaactcactccgggcagaagatacagctgtttactactgtgcaaaggttcattatcattctactgccagctcattggactattggggccagggcaccctcgtaacggtgtcatctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgtaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 451 aa, synthetic construct
  REGION 1..451: note = Synthetic: 2633/VH3 heavy chain ( holes ) peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVHYHSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - DNA, 642 bp, synthetic construct
  misc_feature 1..642: note = Synthetic: 2634/VL1 light chain DNA
  source 1..642: mol_type = other DNA
gatatacagatgacacagtctccatcctccctctccgcgtcagttggtgatagggtcaccattacttgtcgcgctagtcaaggcataagaaaccatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagatataatcgggcgccatataccttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 32 - protein, 214 aa, synthetic construct
  REGION 1..214: note = Synthetic: 2634/VL1 light chain peptide
DIQMTQSPSSLSASVGDRVTITCRASQGIRNHLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 321 bp, synthetic construct
  misc_feature 1..321: note = Synthetic: 2634/VL1, VL DNA
  source 1..321: mol_type = other DNA
gatatacagatgacacagtctccatcctccctctccgcgtcagttggtgatagggtcaccattacttgtcgcgctagtcaaggcataagaaaccatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagatataatcgggcgccatataccttcggccagggtacgaaagtcgaaatcaaa

SEQ ID NO: 34 - protein, 107 aa, synthetic construct
  REGION 1..107: note = Synthetic: 2634/VL1, VL peptide
DIQMTQSPSSLSASVGDRVTITCRASQGIRNHLAWYQQKPGKAPKLLIYAASTLQSGVPSRFSGSGSGTDFTLTISSLQPEDVATYYCQRYNRAPYTFGQGTKVEIK

SEQ ID NO: 35 - DNA, 642 bp, synthetic construct
  misc_feature 1..642: note = Synthetic: 2635/VL2 light chain DNA
  source 1..642: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaacattatcatcgggcgccatataccttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 36 - protein, 214 aa, synthetic construct
  REGION 1..214: note = Synthetic: 2635/VL2 light chain peptide
DIQMTQSPSSLSASVGDRVTITCRASQGIRNYLAWYQQKPGKAPKLLIYAASTLQSGVPSRFSGSGSGTDFTLTISSLQPEDVATYYCQHYHRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 321 bp, synthetic construct
  misc_feature 1..321: note = Synthetic: 2635/VL2, VL DNA
  source 1..321: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaacattatcatcgggcgccatataccttcggccagggtacgaaagtcgaaatcaaa

SEQ ID NO: 38 - protein, 107 aa, synthetic construct
  REGION 1..107: note = Synthetic: 2635/VL2, VL peptide
DIQMTQSPSSLSASVGDRVTITCRASQGIRNYLAWYQQKPGKAPKLLIYAASTLQSGVPSRFSGSGSGTDFTLTISSLQPEDVATYYCQHYHRAPYTFGQGTKVEIK

SEQ ID NO: 39 - DNA, 642 bp, synthetic construct
  misc_feature 1..642: note = Synthetic: 2636/VL3 light chain DNA
  source 1..642: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaacattatcatcgggcgccatatcacttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 40 - protein, 214 aa, synthetic construct
  REGION 1..214: note = Synthetic: 2636/VL3 light chain peptide
DIQMTQSPSSLSASVGDRVTITCRASQGIRNYLAWYQQKPGKAPKLLIYAASTLQSGVPSRFSGSGSGTDFTLTISSLQPEDVATYYCQHYHRAPYHFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 321 bp, synthetic construct
  misc_feature 1..321: note = Synthetic: 2636/VL3, VL DNA
  source 1..321: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaacattatcatcgggcgccatatcacttcggccagggtacgaaagtcgaaatcaaa

SEQ ID NO: 42 - protein, 107 aa, synthetic construct
  REGION 1..107: note = Synthetic: 2636/VL3, VL peptide
DIQMTQSPSSLSASVGDRVTITCRASQGIRNYLAWYQQKPGKAPKLLIYAASTLQSGVPSRFSGSGSGTDFTLTISSLQPEDVATYYCQHYHRAPYHFGQGTKVEIK

SEQ ID NO: 43 - DNA, 642 bp, synthetic construct
  misc_feature 1..642: note = Synthetic: 2637/VL4 light chain DNA
  source 1..642: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcatcattatcatcgggcgccatataccttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 44 - protein, 214 aa, synthetic construct
  REGION 1..214: note = Synthetic: 2637/VL4 light chain peptide
DIQMTQSPSSLSASVGDRVTITCRASQGIRNYLAWYQQKPGKAPKLLIYAASTLQSGVPSRFSGSGSGTDFTLTISSLQPEDVATYYCHHYHRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 321 bp, synthetic construct
  misc_feature 1..321: note = Synthetic: 2637/VL4, VL DNA
  source 1..321: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcatcattatcatcgggcgccatataccttcggccagggtacgaaagtcgaaatcaaa

SEQ ID NO: 46 - protein, 107 aa, synthetic construct
  REGION 1..107: note = Synthetic: 2637/VL4, VL peptide
DIQMTQSPSSLSASVGDRVTITCRASQGIRNYLAWYQQKPGKAPKLLIYAASTLQSGVPSRFSGSGSGTDFTLTISSLQPEDVATYYCHHYHRAPYTFGQGTKVEIK

SEQ ID NO: 47 - DNA, 642 bp, synthetic construct
  misc_feature 1..642: note = Synthetic: 2638/VL5 light chain DNA
  source 1..642: mol_type = other DNA
gatatacagatgacacagtctccatcctccctctccgcgtcagttggtgatagggtcaccattacttgtcgcgctagtcaaggcataagaaaccatcatgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagatataatcgggcgccatataccttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 48 - protein, 214 aa, synthetic construct
  REGION 1..214: note = Synthetic: 2638/VL5 light chain peptide
DIQMTQSPSSLSASVGDRVTITCRASQGIRNHHAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 321 bp, synthetic construct
  misc_feature 1..321: note = Synthetic: 2638/VL5, VL DNA
  source 1..321: mol_type = other DNA
gatatacagatgacacagtctccatcctccctctccgcgtcagttggtgatagggtcaccattacttgtcgcgctagtcaaggcataagaaaccatcatgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagatataatcgggcgccatataccttcggccagggtacgaaagtcgaaatcaaa

SEQ ID NO: 50 - protein, 107 aa, synthetic construct
  REGION 1..107: note = Synthetic: 2638/VL5, VL peptide
DIQMTQSPSSLSASVGDRVTITCRASQGIRNHHAWYQQKPGKAPKLLIYAASTLQSGVPSRFSGSGSGTDFTLTISSLQPEDVATYYCQRYNRAPYTFGQGTKVEIK

SEQ ID NO: 51 - DNA, 642 bp, synthetic construct
  misc_feature 1..642: note = Synthetic: 2639/VL6 light chain DNA
  source 1..642: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagacataatcgggcgccatataccttcggccagggtacgaaagtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 52 - protein, 214 aa, synthetic construct
  REGION 1..214: note = Synthetic: 2639/VL6 light chain peptide
DIQMTQSPSSLSASVGDRVTITCRASQGIRNYLAWYQQKPGKAPKLLIYAASTLQSGVPSRFSGSGSGTDFTLTISSLQPEDVATYYCQRHNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 321 bp, synthetic construct
  misc_feature 1..321: note = Synthetic: 2639/VL6, VL DNA
  source 1..321: mol_type = other DNA
gatatacagatgacacagtctccatcctccctctccgcgtcagttggtgatagggtcaccattacttgtcgcgctagtcaaggcataagaaactatcttgcatggtatcagcagaagcccggcaaagctcccaaactcctcatctacgccgcgtctacactgcagagcggtgtcccatctaggtttagtggaagtgggtctggaacagattttactcttacaatatcttccttgcaacccgaagatgttgcaacctattactgtcaaagacataatcgggcgccatataccttcggccagggtacgaaagtcgaaatcaaa

SEQ ID NO: 54 - protein, 107 aa, synthetic construct
  REGION 1..107: note = Synthetic: 2639/VL6, VL peptide
DIQMTQSPSSLSASVGDRVTITCRASQGIRNYLAWYQQKPGKAPKLLIYAASTLQSGVPSRFSGSGSGTDFTLTISSLQPEDVATYYCQRHNRAPYTFGQGTKVEIK

SEQ ID NO: 55 - protein, 5 aa, synthetic construct
  REGION 1..5: note = Synthetic: Ab1 H-CDR1
DYAMH

SEQ ID NO: 56 - protein, 17 aa, synthetic construct
  REGION 1..17: note = Synthetic: Ab1 H-CDR2
AITWNSGHIDYADSVEG

SEQ ID NO: 57 - protein, 12 aa, synthetic construct
  REGION 1..12: note = Synthetic: Ab1 H-CDR3
VSYLSTASSLDY

SEQ ID NO: 58 - protein, 11 aa, synthetic construct
  REGION 1..11: note = Synthetic: Ab1 L-CDR1
RASQGIRNYLA

SEQ ID NO: 59 - protein, 7 aa, synthetic construct
  REGION 1..7: note = Synthetic: Ab1 L-CDR2
AASTLQS

SEQ ID NO: 60 - protein, 9 aa, synthetic construct
  REGION 1..9: note = Synthetic: Ab1 L-CDR3
QRYNRAPYT

SEQ ID NO: 61 - protein, 12 aa, synthetic construct
  REGION 1..12: note = Synthetic: variant Ab1 H-CDR3 (VH1)
VSYHSTASSLDY

SEQ ID NO: 62 - protein, 12 aa, synthetic construct
  REGION 1..12: note = Synthetic: variant Ab1 H-CDR3 (VH2)
VSYLSTAHHLDY

SEQ ID NO: 63 - protein, 12 aa, synthetic construct
  REGION 1..12: note = Synthetic: variant Ab1 H-CDR3 (VH3)
VHYHSTASSLDY

SEQ ID NO: 64 - protein, 11 aa, synthetic construct
  REGION 1..11: note = Synthetic: variant Ab1 L-CDR1 (VL1)
RASQGIRNHLA

SEQ ID NO: 65 - protein, 11 aa, synthetic construct
  REGION 1..11: note = Synthetic: variant Ab1 L-CDR1 (VL5)
RASQGIRNHHA

SEQ ID NO: 66 - protein, 9 aa, synthetic construct
  REGION 1..9: note = Synthetic: variant Ab1 L-CDR3 (VL2)
QHYHRAPYT

SEQ ID NO: 67 - protein, 9 aa, synthetic construct
  REGION 1..9: note = Synthetic: variant Ab1 L-CDR3 (VL3)
QHYHRAPYH

SEQ ID NO: 68 - protein, 9 aa, synthetic construct
  REGION 1..9: note = Synthetic: variant Ab1 L-CDR3 (VL4)
HHYHRAPYT

SEQ ID NO: 69 - protein, 9 aa, synthetic construct
  REGION 1..9: note = Synthetic: variant Ab1 L-CDR3 (VL6)
QRHNRAPYT

SEQ ID NO: 70 - protein, 121 aa, synthetic construct
  REGION 1..121: note = Synthetic: Ab1 pH switch variant VH
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSHDYWGQGTLVTVSS

SEQ ID NO: 71 - protein, 107 aa, synthetic construct
  REGION 1..107: note = Synthetic: Ab1 pH switch variant VL
DIQMTQSPSSLSASVGDRVTITCRASQGIRNYLAWYQQKPGKAPKLLIYAASTLQSGVPSRFSGSGSGTDFTLTISSLQPEDVATYYCHRHNRAPYTFGQGTKVEIK

SEQ ID NO: 72 - protein, 450 aa, synthetic construct
  REGION 1..450: note = Synthetic: Ab2 HC
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 214 aa, synthetic construct
  REGION 1..214: note = Synthetic: Ab2 LC
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 456 aa, synthetic construct
  REGION 1..456: note = Synthetic: Ab3 HC
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 215 aa, synthetic construct
  REGION 1..215: note = Synthetic: Ab3 LC
EIVLTQSPATLSLSPGERATLSCRASQSVYSYLAWYQQKPGQAPRLLIYDASNRATGIPARFSGSGSGTDFTLTISSLEPZDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 229 aa, synthetic construct
  REGION 1..229: note = Synthetic: Ab4 HC
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 77 - protein, 214 aa, synthetic construct
  REGION 1..214: note = Synthetic: Ab4 LC
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 233 aa, Homo sapiens
  REGION 1..233: note = misc_feature - human TNF-alpha peptide
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 79 - protein, 157 aa, Homo sapiens [sequence omitted]

SEQ ID NO: 80 - DNA, 663 bp, Homo sapiens
  misc_feature 1..663: note = human delta-77-88 TNF-alpha DNA
  source 1..663: mol_type = genomic DNA
atgtcaacagagagtatgatccgcgatgtggaactggccgaagaagcactgcctaagaagactgggggacctcaggggagtagacgctgcctgttcctgtccctgttcagctttctgatcgtggccggcgctaccacactgttctgtctgctgcactttggcgtgatcggcccacagagagaggagtttccccgcgacctgtctctgatctccccactggctcaggctgtggctcatgtggtggctaaccctcaggctgagggacagctgcagtggctgaacaggagggctaatgccctgctggctaacggcgtggagctgagagataatcagctggtggtgccatctgagggcctgtacctgatctattcccaggtgctgttcaagggacagggatgcccaagcacccacgtgctgctgacccatacaatctccaggatcgctgtgagctaccagaccaaggtgaacctgctgagcgccatcaagtctccttgtcagagggagacaccagagggagctgaggctaagccatggtacgagcctatctatctgggcggcgtgtttcagctggagaagggcgaccgcctgtctgccgagatcaatcgccccgactatctggattttgctgaaagtggacaggtgtattttggtatcattgctctg

SEQ ID NO: 81 - protein, 221 aa, Homo sapiens
  REGION 1..221: note = misc_feature - human delta-77-88 TNF-alpha peptide
MSTESMIRDVELAEEALPKKTGGPQGSRRCLFLSLFSFLIVAGATTLFCLLHFGVIGPQREEFPRDLSLISPLAQAVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 82 - protein, 233 aa, Macaca fascicularis
  REGION 1..233: note = misc_feature - cynomolgus TNF-alpha peptide
MSTESMIQDVELAEEALPRKTAGPQGSRRCWFLSLFSFLLVAGAATLFCLLHFGVIGPQREEFPKDPSLISPLAQAVRSSSRTPSDKPVAHVVANPQAEGQLQWLNRRANALVANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 83 - protein, 157 aa, Macaca fascicularis
  REGION 1..157: note = misc_feature - cynomolgus TNF-alpha soluble ECD peptide
VRSSSRTPSDKPVAHVVANPQAEGQLQWLNRRANALVANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 84 - protein, 235 aa, Mus musculus
  REGION 1..235: note = misc_feature - murine TNF-alpha peptide
MSTESMIRDVELAEEALPQKMGGFQNSRRCLCLSLFSFLLVAGATTLFCLLNFGVIGPQRDEKFPNGLPLISSMAQTLTLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 85 - protein, 156 aa, Mus musculus
  REGION 1..156: note = misc_feature - murine TNF-alpha soluble ECD peptide
LRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 86 - protein, 12 aa, synthetic construct
  REGION 1..12: note = Synthetic: Ab1 pH switch variant H-CDR3
VSYLSTASSHDY

SEQ ID NO: 87 - protein, 9 aa, synthetic construct
  REGION 1..9: note = Synthetic: Ab1 pH switch variant L-CDR3
HRHNRAPYT

(19) United States
(12) Patent Application Publication
(10) Pub. No.: US 2025/0154242 A1
(43) Pub. Date: May 15, 2025

(54) ANTI-TNF-ALPHA ANTIBODIES AND COMPOSITIONS

(71) Applicant: Adafre Biosciences, LLC, Encinitas, CA (US)

(72) Inventors: Jeffry D. WATKINS, Encinitas, CA (US); J. Monty WATKINS, La Jolla, CA (US)

(73) Assignee: Adafre Biosciences, LLC, Encinitas, CA (US)

(21) Appl. No.: 18/711,465

(22) PCT Filed: Nov. 18, 2022
(86) PCT No.: PCT/US2022/080097

§ 371 Date: May 17, 2024

Related U.S. Application Data
(60) Provisional application No. 63/356,175, filed on Jun. 28, 2022.
(60) Provisional application No. 63/280,904, filed on Nov. 18, 2021.

(51) Int. Cl. C07K 16/24; A61K 39/00

(52) U.S. Cl. CPC C07K 16/241; A61K 2039/505; C07K 2317/35; C07K 2317/92

(57) ABSTRACT

This invention relates to anti-TNFα antibodies and methods of using them in treating diseases and conditions related to TNFα activity. e.g., autoimmune or inflammatory conditions.

### CROSS-REFERENCE TO RELATED APPLICATIONS

This application claims priority from U.S. Provisional Patent Application 63/280,904, filed Nov. 18, 2021, and United States Provisional Patent Application 63/356,175, filed Jun. 28, 2022. The disclosures of those priority applications are incorporated by reference herein in their entirety.

### SEQUENCE LISTING

The instant application contains a Sequence Listing which has been submitted electronically in XML format and is hereby incorporated by reference in its entirety. The electronic copy of the Sequence Listing, created on Nov. 16, 2022, is named 123314.WO002.xml and is 140,409 bytes in size.

### BACKGROUND OF THE INVENTION

TNFα is a pleiotropic, pro-inflammatory cytokine expressed by cells of the immune system, including monocytes/macrophages (de Waal Malefyt et al., J Exp Med. (1991) 174:1209-20), dendritic cells (DCs) (Ho et al., J Immunol. (2001) 166:1499-506), lymphocytes (Brehm et al., J. Immunol. (2005) 175:5043-49; Fauriat et al., Blood (2010) 115:2167-76; Williamson et al., Proc Natl Acad Sci. USA (1983) 80:5397-401) and neutrophils (Coulthard et al., Clin Exp Immunol. (2012) 170:36-46). It is synthesized as a transmembrane protein on the plasma membrane, and subsequently may be proteolytically processed by TNFα-converting enzyme (TACE), liberating soluble TNFα homotrimer protein (Sedger and McDermott, Cytokine & Growth Factor Reviews (2014) 25:453-72). TNFα is a potent mediator of inflammation and is implicated in the pathogenesis of inflammatory and autoimmune diseases (Kalliolias and Ivashkiv, Nat Rev Rheumatol. (2016) 12:49-62).

TNFα is a well-validated therapeutic target, and multiple TNFα antibodies (infliximab, adalimumab, golimumab, certolizumab) are approved for the treatment of certain rheumatic and inflammatory bowel diseases (IBD). Although the antibodies have dramatically improved the treatment outcome of rheumatic diseases, significant immunogenicity is observed with all four antibodies (van Schouenburg et al., Nat Rev Rheumatol. (2013) 9:164-72). Immunogenicity is associated with lower drug levels, which are associated with discontinuation of treatment, lower efficacy, or treatment failure (Adedokun et al., J. Crohn's Colitis (2017) 11:35-46; Adedokun et al., Inflamm Bowel Dis. (2019) 25:1532-40; Atiqi et al., Frontiers Immunol. (2020) 11:312; Bartelds et al., JAMA (2011) 305:1460-8; Gorovits et al., Clinical & Experimental Immunology (2018) 192:348-65; Jani et al., Ann Rheum Dis (2017) 76:208-13; Kennedy et al., Lancet Gastroenterol. Hepatol. (2019) 4:341-53; Radstake et al., Ann Rheum Dis (2009) 68:1739-45; van Schouenburg et al., supra). Optimization of treatment regimens (e.g., dosage, frequency, co-administration of immunomodulators) diminishes, but does not resolve, the immunogenicity issues (Atiqi et al., supra).

The precise molecular mechanism of the immunogenicity of TNFα antibodies is not clear, and it appears that antibodies directed to TNFα may be inherently prone to generating a greater immune response than antibodies directed to other targets. The FDA-approved TNFα antibodies infliximab (chimeric), certolizumab (humanized), adalimumab (human) and golimumab (human) have varying degrees of protein sequence homology to human antibodies, yet all display significant immunogenicity. By contrast, vedolizumab (anti-α₄β₇) and ustekinumab (anti-IL-12/23), two non-TNFα therapeutic antibodies approved for the treatment of certain rheumatic and inflammatory bowel diseases, bind membrane-associated and soluble targets, respectively, and do not elicit significant immunogenicity (Hanauer et al., J Crohn's Colitis (2019) 14:23-32; Sandborn et al., Gastroenterology (2019) 156: Supplement 1, S-1097, AGA Abstract Tu1718; Van den Berghe et al., J Gastro Hepatol. (2018) 34:1175-81; Wyant et al., J Clin Pharmacol. (2021) 61:1174-81).

Two characteristics of the target protein, TNFα, may contribute to the immunogenicity of the entire class of anti-TNFα antibodies. First, TNFα is expressed as a homotrimer protein, and therefore soluble TNFα can form immune complexes (IC) of varying sizes with antibodies, depending on the relative stoichiometries. Large IC are multivalent lattices of varying antigen-antibody ratios, bind IgG receptors with high avidity, and are internalized into processing pathways that promote cross-presentation of MHC class I and presentation of MHC class II-restricted epitopes (Baker et al., Cell Mol Life Sci (2013) 70:1319-34; Krishna and Nadler, Front Immunol. (2016) 7:21; Weflen et al., Mol Biol Cell (2013) 24:2398-405). Consequently, large IC are potent drivers of immunogenicity, and the pre-formation of IC has long been used as a strategy to drive enhanced immune responses (Terres and Wolins, J Immunol. (1961) 86:361-8; Morrison and Terres, J Immunol. (1966) 96:901-5; Klaus, Immunology (1978) 34:643-52). More recently, a crucial role for IC in immunization against anti-TNFα antibodies in mice was demonstrated (Arnoult et al., J Immunol. (2017) 199:418-24).

The second characteristic of TNFα that potentially contributes to the enhanced immunogenicity of anti-TNFα antibodies is its expression on the plasma membrane of antigen presenting cells of the immune system, including dendritic cells (DC). Membrane-associated TNFα (mTNFα) may allow for the internalization and delivery of TNFα antibodies to the endocytic compartment. Antibody-based targeting of membrane proteins on DC has been exploited as a strategy to induce rapid immune responses (Chen et al., Human Vaccines Immunotherapeutics (2016) 12:612-22; Wang et al., Proc Natl Acad Sci. USA (2000) 96:847-52). Related to this, it was recently demonstrated that antibody bound to mTNFα expressed on dendritic cells was rapidly internalized to the endosomes, trafficked to lysosomes, digested, and the antibody peptides were presented by MHC class II molecules (Deora et al., MABS (2017) 9:680-95). Furthermore, tetanus toxin peptides fused to an anti-TNFα antibody were also presented by DCs, initiating a T cell recall proliferation response (Deora et al., supra).

A less immunogenic TNFα antibody might enable maintenance of more consistent serum antibody levels, have fewer treatment failures, and thus, not require treatment discontinuation or a switch to alternative therapeutic agents.

In view of the critical role of TNFα in the pathogenesis of autoimmune and inflammatory conditions, there is a need for new and improved immune therapies that target TNFα.

### SUMMARY OF THE INVENTION

The present disclosure is directed to novel anti-TNFα antibodies, as well as pharmaceutical compositions comprising one or more of these antibodies, and use of the antibodies and pharmaceutical compositions for treatment of autoimmune and inflammatory conditions. In some embodiments, an antibody of the present disclosure is a variant of a well-characterized, clinically validated anti-TNFα antibody engineered both to prevent the formation of large IC and to enhance its dissociation from TNFα at acidic pH. These characteristics are expected to diminish its trafficking to lysosomes after binding soluble or membrane-associated TNFα, and thus, reduce its immunogenicity. Compared to currently available treatments for autoimmune and inflammatory conditions, including antibody treatments, it is contemplated that the antibodies of the present disclosure may provide a superior clinical response either alone or in combination with another therapeutic for treating autoimmune and/or inflammatory conditions.

In some aspects, the present disclosure provides an anti-TNFα antibody or an antigen-binding portion thereof that binds to the same epitope of human TNFα as a reference antibody comprising:
a) a heavy chain (HC) that comprises the amino acid sequence of SEQ ID NO: 2 and a light chain (LC) that comprises the amino acid sequence of SEQ ID NO: 6;
b) a heavy chain (HC) that comprises the amino acid sequence of SEQ ID NO: 72 and a light chain (LC) that comprises the amino acid sequence of SEQ ID NO: 73;
c) a heavy chain (HC) that comprises the amino acid sequence of SEQ ID NO: 74 and a light chain (LC) that comprises the amino acid sequence of SEQ ID NO: 75; or
d) a heavy chain (HC) that comprises the amino acid sequence of SEQ ID NO: 76 and a light chain (LC) that comprises the amino acid sequence of SEQ ID NO: 77;
wherein said anti-TNFα antibody comprises HC and LC at least 90% identical to the HC and LC of the reference antibody, respectively; and wherein said anti-TNFα antibody or antigen-binding portion is monovalent and has a binding affinity for TNFα that is lower at pH 6.0 than at pH 7.4. In certain embodiments, the anti-TNFα antibody or antigen-binding portion is less immunogenic than said reference antibody. The anti-TNFα antibody may comprise, e.g., a monovalent antigen-binding protein comprising an HC at least 90% identical to the HC of the reference antibody and an LC at least 90% identical to the LC of the reference antibody, and a truncated HC lacking the variable domain and CH1 domain, wherein the antigen-binding protein HC and the truncated HC are capable of dimerization. In some embodiments, the antigen-binding protein HC and the truncated HC comprise knobs-into-holes modifications. For example, the antigen-binding protein HC may comprise mutations T366S, L368A, and Y407A in the CH3 domain, and the truncated HC may comprise the mutation T366W in the CH3 domain, or vice-versa. Additionally or alternatively, the antigen-binding protein HC may comprise the mutation Y349C and the truncated HC may comprise the mutation S354C. Unless otherwise specified, residues are numbered according to the Eu system. In particular embodiments, the anti-TNFα antibody or antigen-binding portion:
a) inhibits TNFα stimulation of monocytes;
b) does not form large immune complexes;
c) binds membrane-associated TNFα;
d) is less immunogenic in vivo than said reference antibody;
e) has a longer half-life in vivo than said reference antibody; or
f) any combination of a)-e).

In some aspects, the present disclosure provides a monovalent anti-TNFα antibody that comprises heavy chain (HC) CDR1-3 and light chain (LC) CDR1-3 comprising:
a) SEQ ID NOs: 55, 56, 57, 58, 59, and 68, respectively;
b) SEQ ID NOs: 55, 56, 63, 58, 59, and 60, respectively;
c) SEQ ID NOs: 55, 56, 62, 58, 59, and 60, respectively;
d) SEQ ID NOs: 55, 56, 57, 58, 59, and 60, respectively;
e) SEQ ID NOs: 55, 56, 61, 58, 59, and 60, respectively;
f) SEQ ID NOs: 55, 56, 57, 64, 59, and 60, respectively;
g) SEQ ID NOs: 55, 56, 57, 58, 59, and 66, respectively;
h) SEQ ID NOs: 55, 56, 57, 58, 59, and 67, respectively;
i) SEQ ID NOs: 55, 56, 57, 65, 59, and 60, respectively;
j) SEQ ID NOs: 55, 56, 57, 58, 59, and 69, respectively;
k) SEQ ID NOs: 55, 56, 61, 64, 59, and 60, respectively;
l) SEQ ID NOs: 55, 56, 61, 58, 59, and 66, respectively;
m) SEQ ID NOs: 55, 56, 61, 58, 59, and 67, respectively;
n) SEQ ID NOs: 55, 56, 61, 58, 59, and 68, respectively;
o) SEQ ID NOs: 55, 56, 61, 65, 59, and 60, respectively;
p) SEQ ID NOs: 55, 56, 61, 58, 59, and 69, respectively;
q) SEQ ID NOs: 55, 56, 62, 58, 59, and 66, respectively;
r) SEQ ID NOs: 55, 56, 62, 58, 59, and 67, respectively;
s) SEQ ID NOs: 55, 56, 62, 58, 59, and 68, respectively;
t) SEQ ID NOs: 55, 56, 62, 65, 59, and 60, respectively;
u) SEQ ID NOs: 55, 56, 63, 58, 59, and 66, respectively;
v) SEQ ID NOs: 55, 56, 63, 58, 59, and 67, respectively;
w) SEQ ID NOs: 55, 56, 63, 58, 59, and 68, respectively;
x) SEQ ID NOs: 55, 56, 63, 65, 59, and 60, respectively;
y) SEQ ID NOs: 55, 56, 63, 58, 59, and 69, respectively; or
z) SEQ ID NOs: 55, 56, 86, 58, 59, and 87, respectively.

In some embodiments, the heavy chain variable domain (VH) and the light chain variable domain (VL) of said antibody comprise:
a) SEQ ID NOs: 4 and 46, respectively;
b) SEQ ID NOs: 28 and 8, respectively;
c) SEQ ID NOs: 22 and 8, respectively;
d) SEQ ID NOs: 4 and 8, respectively;
e) SEQ ID NOs: 16 and 8, respectively;
f) SEQ ID NOs: 4 and 34, respectively;
g) SEQ ID NOs: 4 and 38, respectively;
h) SEQ ID NOs: 4 and 42, respectively;
i) SEQ ID NOs: 4 and 50, respectively;
j) SEQ ID NOs: 4 and 54, respectively;
k) SEQ ID NOs: 16 and 34, respectively;
l) SEQ ID NOs: 16 and 38, respectively;
m) SEQ ID NOs: 16 and 42, respectively;
n) SEQ ID NOs: 16 and 46, respectively;
o) SEQ ID NOs: 16 and 50, respectively;
p) SEQ ID NOs: 16 and 54, respectively;
q) SEQ ID NOs: 22 and 38, respectively;
r) SEQ ID NOs: 22 and 42, respectively;
s) SEQ ID NOs: 22 and 46, respectively;
t) SEQ ID NOs: 22 and 50, respectively;
u) SEQ ID NOs: 28 and 38, respectively;
v) SEQ ID NOs: 28 and 42, respectively;
w) SEQ ID NOs: 28 and 46, respectively;
x) SEQ ID NOs: 28 and 50, respectively;
y) SEQ ID NOs: 28 and 54, respectively; or
z) SEQ ID NOs: 70 and 71, respectively.

In some embodiments, the heavy chain (HC) and the light chain (LC) of said antibody comprise:
a) SEQ ID NOs: 2 and 44, respectively;
b) SEQ ID NOs: 26 and 6, respectively;
c) SEQ ID NOs: 20 and 6, respectively;
d) SEQ ID NOs: 14 and 6, respectively;
e) SEQ ID NOs: 2 and 32, respectively;
f) SEQ ID NOs: 2 and 36, respectively;
g) SEQ ID NOs: 2 and 40, respectively;
h) SEQ ID NOs: 2 and 48, respectively;
i) SEQ ID NOs: 2 and 52, respectively;
j) SEQ ID NOs: 14 and 32, respectively;
k) SEQ ID NOs: 14 and 36, respectively;
l) SEQ ID NOs: 14 and 40, respectively;
m) SEQ ID NOs: 14 and 44, respectively;
n) SEQ ID NOs: 14 and 48, respectively;
o) SEQ ID NOs: 14 and 52, respectively;
p) SEQ ID NOs: 20 and 36, respectively;
q) SEQ ID NOs: 20 and 40, respectively;
r) SEQ ID NOs: 20 and 44, respectively;
s) SEQ ID NOs: 20 and 48, respectively;
t) SEQ ID NOs: 26 and 36, respectively;
u) SEQ ID NOs: 26 and 40, respectively;
v) SEQ ID NOs: 26 and 44, respectively;
w) SEQ ID NOs: 26 and 48, respectively; or
x) SEQ ID NOs: 26 and 52, respectively.

In some embodiments, the monovalent anti-TNFα antibody comprises
a) a monovalent antigen-binding protein that comprises an HC comprising a VH, and an LC comprising a VL, from an above-described VH/VL pair; and
b) a truncated HC lacking the variable domain and CH1 domain;
wherein the antigen-binding protein HC and the truncated HC are capable of dimerization. In certain embodiments, the antigen-binding protein HC and the truncated HC comprise knobs-into-holes modifications. For example, the antigen-binding protein HC may comprise mutations T366S, L368A, and Y407A in the CH3 domain, and the truncated HC may comprise the mutation T366W in the CH3 domain, or vice-versa. Additionally or alternatively, the antigen-binding protein HC may comprise the mutation Y349C and the truncated HC may comprise the mutation S354C. In particular embodiments, the antigen-binding protein HC, the antigen-binding protein LC, and the truncated HC comprise:
a) SEQ ID NOs: 10, 44, and 12, respectively;
b) SEQ ID NOs: 30, 6, and 12, respectively;
c) SEQ ID NOs: 24, 6, and 12, respectively;
d) SEQ ID NOs: 10, 6, and 12, respectively;
e) SEQ ID NOs: 18, 6, and 12, respectively;
f) SEQ ID NOs: 10, 32, and 12, respectively;
g) SEQ ID NOs: 10, 36, and 12, respectively;
h) SEQ ID NOs: 10, 40, and 12, respectively;
i) SEQ ID NOs: 10, 48, and 12, respectively;
j) SEQ ID NOs: 10, 52, and 12, respectively;
k) SEQ ID NOs: 18, 32, and 12, respectively;
l) SEQ ID NOs: 18, 36, and 12, respectively;
m) SEQ ID NOs: 18, 40, and 12, respectively;
n) SEQ ID NOs: 18, 44, and 12, respectively;
o) SEQ ID NOs: 18, 48, and 12, respectively;
p) SEQ ID NOs: 18, 52, and 12, respectively;
q) SEQ ID NOs: 24, 36, and 12, respectively;
r) SEQ ID NOs: 24, 40, and 12, respectively;
s) SEQ ID NOs: 24, 44, and 12, respectively;
t) SEQ ID NOs: 24, 48, and 12, respectively;
u) SEQ ID NOs: 30, 36, and 12, respectively;
v) SEQ ID NOs: 30, 40, and 12, respectively;
w) SEQ ID NOs: 30, 44, and 12, respectively;
x) SEQ ID NOs: 30, 48, and 12, respectively; or
y) SEQ ID NOs: 30, 52, and 12, respectively.

In some embodiments, the monovalent anti-TNFα antibody comprises
a) a single-chain variable fragment (scFv) that comprises an above-described VH/VL pair, linked to an Fc monomer domain; and
b) a truncated HC lacking the variable domain and CH1 domain;
wherein the Fc monomer linked to the scFv, and the truncated HC, are capable of dimerization. The Fc monomer linked to the scFv, and the truncated HC, may be of isotype subclass IgG1. In certain embodiments, the Fc monomer linked to the scFv, and the truncated HC, comprise knobs-into-holes modifications. For example, the Fc monomer linked to the scFv may comprise mutations T366S, L368A, and Y407A in the CH3 domain, and the truncated HC may comprise the mutation T366W in the CH3 domain, or vice-versa. Additionally or alternatively, the Fc monomer linked to the scFv may comprise the mutation Y349C and the truncated HC may comprise the mutation S354C.

The present disclosure also provides a monovalent anti-TNFα antibody that comprises H-CDR1-3 and L-CDR1-3 that comprise SEQ ID NOs: 55, 56, 57, 58, 59, and 68, respectively. In some embodiments, the antibody comprises a VH comprising SEQ ID NO: 4 and a VL comprising SEQ ID NO: 46. In certain embodiments, the antibody comprises an HC comprising SEQ ID NO: 2 and an LC comprising SEQ ID NO: 44. In particular embodiments, the antibody comprises a monovalent antigen-binding protein comprising an HC and an LC, and further comprises a truncated HC lacking the variable domain and CH1 domain, wherein said antigen-binding protein comprises SEQ ID NOs: 10 and 44 and said truncated HC comprises SEQ ID NO: 12, wherein the antigen-binding protein HC and the truncated HC are capable of dimerization.

The present disclosure also provides a monovalent anti-TNFα antibody that comprises H-CDR1-3 and L-CDR1-3 that comprise SEQ ID NOs: 55, 56, 63, 58, 59, and 60, respectively. In some embodiments, the antibody comprises a VH comprising SEQ ID NO: 28 and a VL comprising SEQ ID NO: 8. In certain embodiments, the antibody comprises an HC comprising SEQ ID NO: 26 and an LC comprising SEQ ID NO: 6. In particular embodiments, the antibody comprises a monovalent antigen-binding protein comprising an HC and an LC, and further comprises a truncated HC lacking the variable domain and CH1 domain, wherein said antigen-binding protein comprises SEQ ID NOs: 30 and 6 and said truncated HC comprises SEQ ID NO: 12, wherein the antigen-binding protein HC and the truncated HC are capable of dimerization.

The present disclosure also provides a monovalent anti-TNFα antibody that comprises H-CDR1-3 and L-CDR1-3 that comprise SEQ ID NOs: 55, 56, 62, 58, 59, and 60, respectively. In some embodiments, the antibody comprises a VH comprising SEQ ID NO: 22 and a VL comprising SEQ ID NO: 8. In certain embodiments, the antibody comprises an HC comprising SEQ ID NO: 20 and an LC comprising SEQ ID NO: 6. In particular embodiments, the antibody comprises a monovalent antigen-binding protein comprising an HC and an LC, and further comprises a truncated HC lacking the variable domain and CH1 domain, wherein said antigen-binding protein comprises SEQ ID NOs: 24 and 6 and said truncated HC comprises SEQ ID NO: 12, wherein the antigen-binding protein HC and the truncated HC are capable of dimerization.

In some embodiments, a monovalent anti-TNFα antibody described herein has a binding affinity for human TNFα that is lower at pH 6.0 than at pH 7.4. In certain embodiments, the antibody binds to human TNFα with a KD of 50 nM or less at pH 7.4 and has a k_{dis} of 2e-004 s⁻¹ or greater at pH 6.0.

In some embodiments, a monovalent anti-TNFα antibody described herein
a) inhibits TNFα stimulation of monocytes;
b) does not form large immune complexes;
c) binds membrane-associated TNFα;
d) is less immunogenic in vivo than an antibody comprising an HC that comprises SEQ ID NO: 2 and an LC that comprises SEQ ID NO: 6;
e) has a longer half-life in vivo than an antibody comprising an HC that comprises SEQ ID NO: 2 and an LC that comprises SEQ ID NO: 6; or
f) any combination of a)-e).

The present disclosure also provides a bispecific binding molecule having the binding specificity of an anti-TNFα antibody described herein and the binding specificity of a second, distinct antibody. In some embodiments, the second antibody is an anti-IL17A antibody, an anti-IL23 antibody, or an anti-angiopoietin 2 (Ang2) antibody.

The present disclosure also provides isolated nucleic acid molecule(s) comprising nucleotide sequences that encode the heavy and light chains of an anti-TNFα antibody or an antigen-binding portion thereof as described herein. In some embodiments, the isolated nucleic acid molecule(s) comprise the nucleotide sequences of:
a) SEQ ID NOs: 3 and 45, respectively;
b) SEQ ID NOs: 27 and 7, respectively;
c) SEQ ID NOs: 21 and 7, respectively;
d) SEQ ID NOs: 3 and 7, respectively;
e) SEQ ID NOs: 15 and 7, respectively;
f) SEQ ID NOs: 3 and 33, respectively;
g) SEQ ID NOs: 3 and 37, respectively;
h) SEQ ID NOs: 3 and 41, respectively;
i) SEQ ID NOs: 3 and 49, respectively;
j) SEQ ID NOs: 3 and 53, respectively;
k) SEQ ID NOs: 15 and 33, respectively;
l) SEQ ID NOs: 15 and 37, respectively;
m) SEQ ID NOs: 15 and 41, respectively;
n) SEQ ID NOs: 15 and 45, respectively;
o) SEQ ID NOs: 15 and 49, respectively;
p) SEQ ID NOs: 15 and 53, respectively;
q) SEQ ID NOs: 21 and 37, respectively;
r) SEQ ID NOs: 21 and 41, respectively;
s) SEQ ID NOs: 21 and 45, respectively;
t) SEQ ID NOs: 21 and 49, respectively;
u) SEQ ID NOs: 27 and 37, respectively;
v) SEQ ID NOs: 27 and 41, respectively;
w) SEQ ID NOs: 27 and 45, respectively;
x) SEQ ID NOs: 27 and 49, respectively; or
y) SEQ ID NOs: 27 and 53, respectively.

In some embodiments, the isolated nucleic acid molecule(s) comprise the nucleotide sequences of:
a) SEQ ID NOs: 9, 43, and 11, respectively;
b) SEQ ID NOs: 29, 5, and 11, respectively;
c) SEQ ID NOs: 23, 5, and 11, respectively;
d) SEQ ID NOs: 9, 5, and 11, respectively;
e) SEQ ID NOs: 17, 5, and 11, respectively;
f) SEQ ID NOs: 9, 31, and 11, respectively;
g) SEQ ID NOs: 9, 35, and 11, respectively;
h) SEQ ID NOs: 9, 39, and 11, respectively;
i) SEQ ID NOs: 9, 47, and 11, respectively;
j) SEQ ID NOs: 9, 51, and 11, respectively;
k) SEQ ID NOs: 17, 31, and 11, respectively;
l) SEQ ID NOs: 17, 35, and 11, respectively;
m) SEQ ID NOs: 17, 39, and 11, respectively;
n) SEQ ID NOs: 17, 43, and 11, respectively;
o) SEQ ID NOs: 17, 47, and 11, respectively;
p) SEQ ID NOs: 17, 51, and 11, respectively;
q) SEQ ID NOs: 23, 35, and 11, respectively;
r) SEQ ID NOs: 23, 39, and 11, respectively;
s) SEQ ID NOs: 23, 43, and 11, respectively;
t) SEQ ID NOs: 23, 47, and 11, respectively;
u) SEQ ID NOs: 29, 35, and 11, respectively;
v) SEQ ID NOs: 29, 39, and 11, respectively;
w) SEQ ID NOs: 29, 43, and 11, respectively;
x) SEQ ID NOs: 29, 47, and 11, respectively; or
y) SEQ ID NOs: 29, 51, and 11, respectively.

In some embodiments, the isolated nucleic acid molecule(s) comprise the nucleotide sequences of:
a) SEQ ID NOs: 1 and 43, respectively;
b) SEQ ID NOs: 15 and 5, respectively;
c) SEQ ID NOs: 9 and 5, respectively;
d) SEQ ID NOs: 13 and 5, respectively;
e) SEQ ID NOs: 1 and 31, respectively;
f) SEQ ID NOs: 1 and 35, respectively;
g) SEQ ID NOs: 1 and 39, respectively;
h) SEQ ID NOs: 1 and 47, respectively;
i) SEQ ID NOs: 1 and 51, respectively;
j) SEQ ID NOs: 13 and 31, respectively;
k) SEQ ID NOs: 13 and 35, respectively;
l) SEQ ID NOs: 13 and 39, respectively;
m) SEQ ID NOs: 13 and 43, respectively;
n) SEQ ID NOs: 13 and 47, respectively;
o) SEQ ID NOs: 13 and 51, respectively;
p) SEQ ID NOs: 9 and 35, respectively;
q) SEQ ID NOs: 9 and 39, respectively;
r) SEQ ID NOs: 9 and 43, respectively;
s) SEQ ID NOs: 9 and 47, respectively;
t) SEQ ID NOs: 15 and 35, respectively;
u) SEQ ID NOs: 15 and 39, respectively;
v) SEQ ID NOs: 15 and 43, respectively;
w) SEQ ID NOs: 15 and 47, respectively; or
x) SEQ ID NOs: 15 and 51.

The present disclosure also provides vector(s) comprising isolated nucleic acid molecule(s) described herein. In some embodiments, the vector(s) further comprise expression control sequence(s) linked operatively to the isolated nucleic acid molecule(s).

The present disclosure also provides a host cell comprising a nucleotide sequence that encodes the heavy chain sequence(s), and/or a nucleotide sequence that encodes the light chain sequence, of an anti-TNFα antibody or antigen-binding portion thereof described herein. In some embodiments, the host cell comprises nucleotide sequences encoding both the heavy chain sequence(s) and the light chain sequence. In some embodiments, the host cell comprises isolated nucleic acid molecule(s) described herein.

The present disclosure also provides a method for producing an anti-TNFα antibody or an antigen-binding portion thereof described herein, comprising providing a host cell described herein, culturing the host cell under conditions suitable for expression of the antibody or portion, and isolating the resulting antibody or portion.

The present disclosure also provides a pharmaceutical composition comprising an anti-TNFα antibody or an antigen-binding portion thereof described herein and a pharmaceutically acceptable excipient. In some embodiments, the pharmaceutical composition may comprise an additional therapeutic agent.

The present disclosure also provides a method for treating a condition (e.g., an autoimmune or inflammatory condition) in a patient in need thereof, comprising administering to the patient a therapeutically effective amount of an anti-TNFα antibody or an antigen-binding portion thereof described herein. The anti-TNFα antibody or antigen-binding portion may also be used in the manufacture of a medicament for treating the condition, or may be for use in treating the condition. In some embodiments, the condition is an autoimmune or inflammatory condition selected from rheumatoid arthritis, psoriatic arthritis, plaque psoriasis, ankylosing spondylitis, axial spondyloarthritis, Crohn's disease, ulcerative colitis, hidradenitis suppurativa, polyarticular juvenile idiopathic arthritis, panuveitis, and Alzheimer's disease. In some embodiments, the patient may be treated with an additional therapeutic agent (e.g., methotrexate).

The present disclosure also provides a kit comprising an anti-TNFα antibody or an antigen-binding portion thereof described herein. In some embodiments, the kit is for use in a treatment described herein.

The present disclosure also provides an article of manufacture comprising an anti-TNFα antibody or an antigen-binding portion thereof described herein. In some embodiments, the article of manufacture is suitable for treating a condition (e.g., a condition described herein, such as an autoimmune or inflammatory condition described herein) in a patient in need thereof. In some embodiments, the treatment is as described herein.

Other features, objectives, and advantages of the invention are apparent in the detailed description that follows. It should be understood, however, that the detailed description, while indicating embodiments and embodiments of the invention, is given by way of illustration only, not limitation. Various changes and modifications within the scope of the invention will become apparent to those skilled in the art from the detailed description.

### BRIEF DESCRIPTION OF THE DRAWINGS

FIG. 1. SDS-PAGE of Ab1 and monovalent Ab1 (AF-M2630). Ab1 and AF-M2630 were transiently expressed in HEK-293 cells, and purified by protein A chromatography, and 2 μg of each antibody was loaded on an SDS-PAGE in the absence (A) or presence (B) of reducing agent.

FIG. 2. Ab1 and AF-M2630 (monovalent Ab1) bind TNFα with high affinity. The binding of Ab1 (closed circles) and AF-M2630 (open circles) to TNFα following (A) brief and (B) prolonged wash steps at pH 7.4 was quantitated.

FIG. 3. AF-M2630 prevents the formation of large precipitating IC. The ability of Ab1 and AF-M2630 (monovalent Ab1) to form large precipitating IC was evaluated using an Ouchterlony double diffusion assay. Human TNFα (right) or anti-human Fc antibody (left) was loaded in the central well of an Ouchterlony agarose gel. Ab1, AF-M2630, a second monovalent anti-TNFα antibody (AF-M2633), and control IgG were loaded in the surrounding wells. Following diffusion for 16 h at 37° C., protein was detected with SimplyBlue SafeStain. Precipitation of protein complexes generates dark protein staining (*).

FIG. 4. AF-M2630 forms small IC with TNFα, regardless of the stoichiometry. IC formed with Ab1 and AF-M2630 were characterized by analytical size exclusion chromatography (SEC). (A) Ab1, (B) AF-M2630, Ab1-TNFα mixtures incubated overnight at 4° C. at a (C) 10:1 and (E) 1:1 stoichiometry, and AF-M2630-TNFα mixtures incubated overnight at 4° C. at a (D) 10:1 and (F) 1:1 stoichiometry were applied to a TSKgel UP-SW3000 column and protein retention time was monitored at A290. Protein peaks are highlighted corresponding to small IC (a single homotrimer TNFα complexed with 1-3 antibodies) eluting with retention times consistent with a MW<550 kDa, and large IC (comprising 2 or more TNFα molecules crosslinked by three or more antibodies) eluting with retention times consistent with a MW>669 kDa. Molecular weight standards thyroglobulin dimer (1,338 kDa), thyroglobulin (669 kDa), ferritin (440 kDa), aldolase (158 kDa) and conalbumin (75 kDa) were also resolved and the elution times are indicated with hash marks on the x-axis (E and F). Conalbumin standard was not included with the AF-M2630 experiment.

FIG. 5. Binding of Ab1 and monovalent pH switch variants to human TNFα following prolonged wash at pH 7.4 or pH 6.0. Binding of Ab1 and the variants to human TNFα was characterized by ELISA. Following binding of the antibodies to TNFα, the plates were subjected to a prolonged wash at (A) pH 7.4 or at (B) pH 6.0.

FIG. 6. AF-M2644 and AF-M2648 do not bind immobilized TNFα in ELISA format. (A) No detectable binding of AF-M2648 (squares) was detected following pH 7.4 wash. By contrast, Ab1 (with abrogated effector function) binds tightly with an EC50=6.312E-11. (B) Little binding of AF-M2644 (triangles) was detected following pH 7.4 wash. By contrast, Ab1 (EC50=9.759E-11) and AF-M2630 (EC50=1.345E-10) bind tightly.

FIG. 7. Binding of Ab1 and variants to human TNFα following pH 7.4 wash. The binding of: (A) AF-M2645 (VH1/VL6), AF-M2646 (VH2/VL2), AF-M2649 (VH2/VL5), AF-M2653 (VH3/VL5) and AF-M2654 (VH3/VL6), (B) Ab1, AF-M2641 (VH1/VL2), AF-M2643 (VH1/VL4), AF-M2650 (VH3/VL2), AF-M2652 (VH3/VL4) and (C) AF-M2634 (VL1), AF-M2635 (VL2), AF-M2639 (VL6), AF-M2640 (VH1/VL1), and Ab1 was quantitated following a pH 7.4 wash.

FIG. 8. Binding of AF-M2630 (monovalent Ab1) and pH switch variants to human TNFα following a prolonged wash at pH 7.4 or pH 6.0. The binding of AF-M2632 (VH2), AF-M2633 (VH3), AF-M2636 (VL3), AF-M2637 (VL4), AF-M2642 (VH1/VL3) and AF-M2651 (VH3/VL3) to human TNFα was characterized by ELISA. Following binding of the antibodies to TNFα the plates were subjected to a prolonged wash at (A) pH 7.4 or at (B) pH 6.0.

FIG. 9. Binding of Ab1 and bivalent pH switch variants to human TNFα following a prolonged wash at pH 7.4 or pH 6.0. Binding of Ab1 and the variants to human TNFα was characterized by ELISA. Following binding of the antibodies to TNFα the plates were subjected to a prolonged wash at (A) pH 7.4 or at (B) pH 6.0.

FIG. 10. Binding of soluble human TNFα to immobilized Ab1 and variants. Biotinylated human TNFα was titrated against Ab1 and variants. The binding of the bivalent (closed symbols) and monovalent (open symbols) format of Ab1 (circles) and the pH switch variants AF-2631 (squares), AF-2637 (triangles), and AF-M2632 (inverted triangles), and AF-M2633 (diamonds) was characterized. Binding was quantitated following a prolonged wash at (A) pH 7.4 or (B) at pH 6.0.

FIG. 11. Demonstration of the binding of Ab1 and variants to mTNFα-expressing CHO cells by flow cytometry. (A) Ab1, (B) AF-M2630, (C) AF-B2631, (D) AF-M2631, (E) AF-B2637, and (F) AF-M2637 were incubated with the mTNFα-expressing CHO cell line at 10 μg/mL (1 μg/500,000 cells), and binding was detected with an FITC-labeled secondary antibody (black histograms). An isotype-matched, irrelevant humanized IgG1 (HUC2-MRVA) was used as a negative control (gray histogram).

FIG. 12. Ab1 and variants bind to mTNFα-expressing CHO cells in an ELISA format. Recombinant CHO cells expressing mTNFα were plated on a 96-well microtiter cell culture plate. Subsequently, Ab1 and variants were serially diluted and binding was quantitated following the addition of goat anti-human IgG Fc-HRP.

FIG. 13. HEK-Blue cell dose response to human and cynomolgus TNFα. Human (open circles) or cynomolgus (closed circles) TNFα was titrated on HEK-Blue cells and SEAP expression was quantitated 20 h later. The EC50 values are based on human TNFα MW=54 kDa and cynomolgus TNFα MW=52.5 kDa.

FIG. 14. Inhibition of human TNFα stimulation of HEK-Blue cells. Ab1 and variants were titrated with 5 pM human TNFα and the mixture was incubated with the cells for 20 h. Stimulation of the cells was assessed by quantitating secretion of the reporter gene product, SEAP, in the culture supernatant. Bivalent constructs (closed symbols) and the corresponding monovalent construct (open symbols) of Ab1 (circles), pH switch variant AF-2631 (VH1, squares) and pH switch variant AF-2637 (VL4, triangles) were characterized.

FIG. 15. Comparison of TNFα sensitivity of THP1-Blue™ NF-kB cells versus HEK-Blue™ TNFα cells. Recombinant human TNFα was titrated on both cell lines and expression of SEAP in the culture supernatant was quantitated as described in the Materials and Methods. The EC50 values are based on human TNFα MW=52.2 kDa.

FIG. 16. Inhibition of human TNFα stimulation of THP1-Blue™ TNFα cells. Ab1 and variants were titrated with 10 pM human TNFα and the mixture was incubated with the cells for 20 h. Stimulation of the cells was assessed by quantitating secretion of the reporter gene product, SEAP, in the culture supernatant. Bivalent constructs (closed symbols) and the corresponding monovalent construct (open symbols) of Ab1 (circles), pH switch variant AF-2631 (VH1, squares) and pH switch variant AF-2637 (VL4, triangles) were characterized.

FIG. 17. Inhibition of human TNFα stimulation of THP1-Blue™ TNFα cells. Ab1 and variants were titrated with 10 pM human TNFα and the mixture was incubated with the cells for 20 h. Stimulation of the cells was assessed by quantitating secretion of the reporter gene product, SEAP, in the culture supernatant. Ab1 (open circles) and the monovalent constructs AF-M2630 (closed circles), AF-M2631 (squares), AF-M2632 (triangles), AF-M2633 (inverted triangles) and AF-M2637 (diamonds) were characterized.

FIG. 18. Binding of soluble murine TNFα to immobilized Ab1 and variants. Biotinylated murine TNFα was titrated against Ab1 and variants. The binding of the bivalent (closed symbols) and monovalent (open symbols) format of Ab1 (circles) and the pH switch variants AF-2631 (squares) and AF-2637 (triangles) was characterized. Binding was quantitated following a (A) brief or (B) prolonged wash at pH 7.4 or following a (C) prolonged wash at pH 6.0.

FIG. 19. Binding of soluble murine TNFα to immobilized Ab1 and variants. Biotinylated murine TNFα was titrated against Ab1 and variants. The binding of the bivalent (closed symbols) and monovalent (open symbols) format of Ab1 (circles) and the pH switch variants AF-2631 (squares), AF-2637 (triangles), AF-M2632 (inverted triangles), and AF-M2633 (diamonds) was characterized. Binding was quantitated following a prolonged wash at (A) pH 7.4 or (B) at pH 6.0.

FIG. 20. Sequence alignment of the soluble domains of human and murine TNFα. The soluble domain of murine TNFα (SEQ ID NO: 85) is 79% (124/157) identical to the soluble domain of human TNFα (SEQ ID NO: 79) and contains one gap. Human residues in bold indicate epitope residues of Ab1 Fab (Hu et al., J Biol Chem. (2013) 288:27,059-67). Murine residues that differ from the corresponding to the human epitope residues are also indicated in bold (H20, gap at residue 72, Y73, L111, V136, L138, and K140).

FIG. 21. AF-M2637 is less immunogenic than Ab1 in mice. The murine anti-drug antibody (ADA) response against Ab1 and variants was characterized. A single intravenous 4 mg/kg dose of (A) Ab1, (B) AF-M2630, (C) AF-B2631, (D) AF-M2631, (E) AF-B2637 or (F) AF-M2637 was administered to mice on day 0. Serum was collected on days 1, 3, 6, 7, 9, 14, and 21 and was screened by ELISA for IgG (black circles) or IgM (open circles) antibodies against the test article. Each serum sample was diluted 1:100 and assayed in duplicate and each data point corresponds to mean±SEM of absorbance values from three mice.

FIG. 22. AF-M2637 has a slower elimination than Ab1 in mice. The serum concentration of Ab1 and variants was characterized following a single intravenous injection. Mice were administered a single, intravenous 4 mg/kg dose of (A) Ab1, (B) AF-M2630, (C) AF-B2631, (D) AF-M2631, (E) AF-B2637 or (F) AF-M2637 and serum antibody levels were quantitated by ELISA on days 1, 3, 6, 7, 9, 14, and 21. Each serum sample was diluted 1:10 and then serially 3-fold and all values obtained from the linear portion of the standard curve were averaged to determine the serum antibody concentration for each mouse. Each data point corresponds to the mean±SEM from three mice.

FIG. 23. Ab1 and pH switch variants bind cynomolgus TNFα. Ab1 and variants were immobilized on a 96-well plate. Subsequently, biotinylated cynomolgus TNFα was titrated and the plate was washed with either (A) PBS, 0.05% Tween 20, pH 7.4 or with (B) PBS, 0.05% Tween 20, pH 7.4 and binding was quantitated using neutravidin HRP.

FIG. 24. Sequence alignment of the soluble domains of human and cynomolgus TNFα. The soluble domain of cynomolgus TNFα (SEQ ID NO: 83) is 97% (153/157) identical to the soluble domain of human TNFα (SEQ ID NO: 79). Human residues in bold indicate epitope residues of Ab1 Fab (Hu et al., supra). Cynomolgus residues that differ from those corresponding to the human epitope residues are also indicated in bold (N72 and L138).

FIG. 25. The pH switch variants inhibit cynomolgus TNFα stimulation of HEK-Blue cells. Ab1 and variants were titrated with 5 pM cynomolgus TNFα and the mixture was incubated with the cells for 20-24 h. Stimulation of the cells was assessed by quantitating secretion of the reporter gene product, SEAP, in the culture supernatant. Bivalent constructs (closed symbols) and the corresponding monovalent construct (open symbols) of Ab1 (circles), pH switch variant AF-2631 (squares) and pH switch variant AF-2637 (triangles) were characterized.

### DETAILED DESCRIPTION OF THE INVENTION

The present disclosure provides novel anti-human TNFα antibodies and antigen-binding portions thereof that can be used to treat autoimmune and/or inflammatory conditions. Unless otherwise stated, as used herein, “TNFα” refers to human TNFα. A human TNFα polypeptide sequence is shown below:

(SEQ ID NO: 78)
MSTESMIRDVELAEEALPKKTGGPQGSRRCLFLSLESFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

The term “antibody” (Ab) or “immunoglobulin” (Ig), as used herein, may refer to a tetramer comprising two heavy (H) chains (about 50-70 kDa) and two light (L) chains (about 25 kDa) interconnected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. In some embodiments, an antibody described herein may be a bivalent antibody. The term “bivalent antibody,” as used herein, refers to an antibody with two antigen-binding sites. In some embodiments, an antibody described herein may be a monovalent antibody comprising less than two HCs and two LCs (e.g., comprising a single VH and VL, or HC and LC, from an anti-TNFα antibody). The term “monovalent antibody,” as used herein, refers to an antibody with one antigen-binding site.

The assignment of amino acid numbers, and/or of FR and CDR regions, in the heavy or light chain may be in accordance with IMGT® definitions (Lefranc et al., Dev Comp Immunol. (2003) 27(1):55-77), Eu numbering, or the definitions of Kabat, Sequences of Proteins of Immunological Interest (National Institutes of Health, Bethesda, MD (1987 and 1991); Chothia & Lesk, J Mol Biol. (1987) 196:901-17; Chothia et al., Nature (1989) 342:878-83; MacCallum et al., J Mol Biol. (1996) 262:732-45; or Honegger and Plückthun, J Mol Biol. (2001) 309 (3): 657-70 (“AHo” numbering).

In some embodiments, an antibody or antigen-binding portion thereof of the present disclosure is an isolated antibody or antigen-binding portion. The term “isolated protein”, “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between an antigen and an antibody or an antigen-binding fragment thereof, or a related molecule such as a bispecific binding molecule. The intrinsic attractiveness of the antibody for the antigen is typically expressed as the binding affinity equilibrium constant (K_{D}) of a particular antibody-antigen interaction. An antibody or antigen-binding portion is said to specifically bind to an antigen when the K_{D} is ≤1 μM, e.g., ≤100 nM or ≤10 nM. A K_{D} binding affinity constant can be measured, e.g., by surface plasmon resonance (BIAcore™) or Bio-Layer Interferometry, for example using the IBIS MX96 SPR system from IBIS Technologies, the Carterra LSA SPR platform, or the Octet™ system from ForteBio.

The term “epitope” as used herein refers to a portion (determinant) of an antigen that specifically binds to an antibody or an antigen-binding portion thereof. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., TNFα) or a relevant portion thereof, then screening for binding to the epitope.

One can determine whether an antibody binds to the same epitope of TNFα as or competes for binding with an antibody described herein by using methods known in the art, including, without limitation, competition assays, epitope binning, and alanine scanning. In some embodiments, one allows an antibody described herein to bind to TNFα under saturating conditions, and then measures the ability of the test antibody to bind to said antigen. If the test antibody is able to bind to said antigen at the same time as the reference antibody, then the test antibody binds to a different epitope than the reference antibody. However, if the test antibody is not able to bind to the antigen at the same time, then the test antibody binds to the same epitope, an overlapping epitope, or an epitope that is in close proximity to the epitope bound by the antibody described herein. This experiment can be performed using, e.g., ELISA, RIA, BIACORE™, SPR, Bio-Layer Interferometry or flow cytometry. To test whether an antibody described herein cross-competes with another antibody for binding to TNFα, one may use the competition method described above in two directions, i.e., determining if the known antibody blocks the test antibody and vice versa. Such cross-competition experiments may be performed, e.g., using an IBIS MX96 SPR instrument or the Octet™ system.

The term “antigen-binding portion” or “antigen-binding fragment” of an antibody, as used herein, refers to one or more portions or fragments of an antibody that retain the ability to specifically bind to an antigen (e.g., human TNFα, or a portion thereof). It has been shown that certain fragments of a full-length antibody can perform the antigen-binding function of the antibody. Examples of binding fragments encompassed within the term “antigen-binding portion” include (i) a Fab fragment: a monovalent fragment consisting of the VL, VH, CL and CH1 domains; (ii) a F(ab′)2 fragment: a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR) capable of specifically binding to an antigen. Furthermore, although the two domains of the Fv fragment, VL and VH, are encoded by separate genes, they can be joined, using recombinant methods, by a synthetic linker that enables them to be made as a single protein chain in which the VL and VH domains pair to form monovalent molecules known as single chain variable fragments (scFvs). Also within the present disclosure are antigen-binding molecules comprising a VH and/or a VL. In the case of a VH, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites. The present disclosure also contemplates antigen-binding portions of the anti-TNFα antibodies described herein, wherein the antigen-binding portions retain the functional properties of the cognate antibodies. Such antigen-binding portions may be used where the cognate antibody is used.

### Anti-TNFα Antibodies

The present disclosure is based on the discovery of strategies for engineering less immunogenic forms of therapeutic anti-TNFα antibodies. Such engineered antibodies may maintain more consistent serum antibody levels and have greater or more prolonged therapeutic efficacy compared to the parent antibodies. In some embodiments, the antibodies of the present disclosure are engineered to prevent the formation of large immune complexes (IC), to enhance their dissociation from TNFα at acidic pH, or both. As used herein, “large IC” refers to immune complexes that comprise ≥2 TNFα trimers and ≥3 antibodies or antigen-binding portions. In certain embodiments, the antibodies are monovalent. Without wishing to be bound by theory, it is contemplated that monovalency reduces or eliminates the formation of large IC via antibody-mediated cross-linking of TNFα. In certain embodiments, the antibodies have a pH-sensitive antigen binding function (“pH switch”). The pH switch allows the antibody to bind and neutralize serum (soluble) and membrane-associated TNFα at physiological pH (e.g., ˜pH 7.4), while also enabling dissociation following internalization into the acidic endosomal environment (e.g., ˜pH 6.0). The dissociated antibody may then be recycled to the cell surface via the FcRn, while the antigen is trafficked to the lysosomes for degradation. In particular embodiments, the antibodies of the present disclosure are monovalent and incorporate a pH switch.

In some embodiments, the present disclosure provides an engineered anti-TNFα antibody or an antigen-binding portion thereof that binds to the same epitope of human TNFα as a reference antibody comprising
a) a heavy chain (HC) that comprises the amino acid sequence of SEQ ID NO: 2 and a light chain (LC) that comprises the amino acid sequence of SEQ ID NO: 6 (“Ab1”);
b) a heavy chain (HC) that comprises the amino acid sequence of SEQ ID NO: 72 and a light chain (LC) that comprises the amino acid sequence of SEQ ID NO: 73 (“Ab2”);
c) a heavy chain (HC) that comprises the amino acid sequence of SEQ ID NO: 74 and a light chain (LC) that comprises the amino acid sequence of SEQ ID NO: 75 (“Ab3”); or
d) a heavy chain (HC) that comprises the amino acid sequence of SEQ ID NO: 76 and a light chain (LC) that comprises the amino acid sequence of SEQ ID NO: 77 (“Ab4”), wherein said anti-TNF antibody or antigen-binding portion is less immunogenic than the reference antibody, wherein the anti-TNFα antibody or antigen-binding portion
i) is monovalent;
ii) has a binding affinity for TNFα that is lower at pH 6.0 than at pH 7.4; or
iii) i) and ii).

In particular embodiments, the anti-TNFα antibody comprises HC and LC at least 90% identical to the HC and LC of the reference antibody, respectively. For example, in some embodiments, the anti-TNFα antibody may comprise no more than one, two, three, four, or five amino acid substitutions from the reference antibody (which in certain embodiments may be in H-CDR1, H-CDR2, H-CDR3, L-CDR1, L-CDR2, L-CDR3, or any combination thereof).

In some embodiments, an anti-TNFα antibody or antigen-binding portion thereof of the present disclosure is engineered from parent anti-TNFα antibody “Ab1,” which comprises the amino acid sequences shown below (variable domains italicized, CDRs underlined):

Ab1 HC
(SEQ ID NO: 2)
EVQLVESGGGLVQPGRSLRLSCAASGFTEDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVMHEALHNHYTQKSLSLSPG
K
Ab1 LC
(SEQ ID NO: 6)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

In some embodiments, the anti-TNFα antibody or antigen-binding portion thereof engineered from Ab1
i) is monovalent;
ii) has a binding affinity for TNFα that is lower at pH 6.0 than at pH 7.4; or
iii) i) and ii).

In particular embodiments, the anti-TNFα antibody or antigen-binding portion thereof is monovalent and has a binding affinity for TNFα that is lower at pH 6.0 than at pH 7.4.

In some embodiments, the anti-TNFα antibody or antigen-binding portion has VH and VL amino acid sequences that comprise, in total, at least one, two, three, four, or five amino acid substitutions from the VH and VL amino acid sequences of Ab1. In certain embodiments, the VH and VL amino acid sequences comprise, in total, one amino acid substitution from the VH and VL amino acid sequences of Ab1. In certain embodiments, the VH and VL amino acid sequences comprise, in total, two amino acid substitutions from the VH and VL amino acid sequences of Ab1. In certain embodiments, the VH and VL amino acid sequences comprise, in total, three amino acid substitutions from the VH and VL amino acid sequences of Ab1. In particular embodiments, the amino acid substitutions may alter the binding affinity of the antibody or portion at certain pH values; for example, the altered antibody or portion may have a binding affinity for TNFα that is reduced at a lower pH (e.g., pH 6.0) compared to a higher pH (e.g., pH 7.4). In some embodiments, the EC50 for binding at the lower pH may be increased by at least 2-, 5-, 10-, 15-, 20-, 25-, 30-, 50-, 75-, 100-, 500-, 1000-, 2000-, or 4000-fold compared to the binding affinity at the higher pH.

In some embodiments, the amino acid substitution(s) are in the FRs, or the FRs and the CDRs, of the anti-TNFα antibody or antigen-binding portion. In some embodiments, the amino acid substitution(s) are in the CDRs of the anti-TNFα antibody or antigen-binding portion. In certain embodiments, the amino acid substitution(s) are in H-CDR3, L-CDR1, L-CDR3, or any combination thereof (e.g., H-CDR3 and L-CDR1, H-CDR3 and L-CDR3, or H-CDR3, L-CDR1, and L-CDR3). The CDRs may be delineated by the Kabat, Chothia, IMGT, contact, or AHo method, or any combination thereof. In particular embodiments, the CDRs are delineated as shown in the Ab1 sequences above (SEQ ID NOs: 2 and 6).

In certain embodiments, the anti-TNFα antibody or portion comprises an H-CDR1 comprising SEQ ID NO: 55; an H-CDR2 comprising SEQ ID NO: 56; an H-CDR3 comprising a sequence selected from SEQ ID NOs: 57, 61, 62, 63, and 86; an L-CDR1 comprising a sequence selected from SEQ ID NOs: 58, 64, and 65; an L-CDR2 comprising SEQ ID NO: 59; and an L-CDR3 comprising a sequence selected from SEQ ID NOs: 60, 66, 67, 68, 69, and 87. In particular embodiments, the anti-TNFα antibody or portion comprises H-CDR1-3 and L-CDR1-3 that comprise:
a) SEQ ID NOs: 55, 56, 57, 58, 59, and 68, respectively;
b) SEQ ID NOs: 55, 56, 63, 58, 59, and 60, respectively;
c) SEQ ID NOs: 55, 56, 62, 58, 59, and 60, respectively;
d) SEQ ID NOs: 55, 56, 57, 58, 59, and 60, respectively;
e) SEQ ID NOs: 55, 56, 61, 58, 59, and 60, respectively;
f) SEQ ID NOs: 55, 56, 57, 64, 59, and 60, respectively;
g) SEQ ID NOs: 55, 56, 57, 58, 59, and 66, respectively;
h) SEQ ID NOs: 55, 56, 57, 58, 59, and 67, respectively;
i) SEQ ID NOs: 55, 56, 57, 65, 59, and 60, respectively;
j) SEQ ID NOs: 55, 56, 57, 58, 59, and 69, respectively;
k) SEQ ID NOs: 55, 56, 61, 64, 59, and 60, respectively;
l) SEQ ID NOs: 55, 56, 61, 58, 59, and 66, respectively;
m) SEQ ID NOs: 55, 56, 61, 58, 59, and 67, respectively;
n) SEQ ID NOs: 55, 56, 61, 58, 59, and 68, respectively;
o) SEQ ID NOs: 55, 56, 61, 65, 59, and 60, respectively;
p) SEQ ID NOs: 55, 56, 61, 58, 59, and 69, respectively;
q) SEQ ID NOs: 55, 56, 62, 58, 59, and 66, respectively;
r) SEQ ID NOs: 55, 56, 62, 58, 59, and 67, respectively;
s) SEQ ID NOs: 55, 56, 62, 58, 59, and 68, respectively;
t) SEQ ID NOs: 55, 56, 62, 65, 59, and 60, respectively;
u) SEQ ID NOs: 55, 56, 63, 58, 59, and 66, respectively;
v) SEQ ID NOs: 55, 56, 63, 58, 59, and 67, respectively;
w) SEQ ID NOs: 55, 56, 63, 58, 59, and 68, respectively;
x) SEQ ID NOs: 55, 56, 63, 65, 59, and 60, respectively;
y) SEQ ID NOs: 55, 56, 63, 58, 59, and 69, respectively; or
z) SEQ ID NOs: 55, 56, 86, 58, 59, and 87, respectively.

In certain embodiments, the anti-TNFα antibody or antigen-binding portion thereof comprises a VH comprising a sequence selected from SEQ ID NOs: 4, 16, 22, 28, and 70 and a VL comprising a sequence selected from SEQ ID NOs: 8, 34, 38, 42, 46, 50, 54, and 71. In particular embodiments, the anti-TNFα antibody or portion comprises a VH and VL that comprise:
a) SEQ ID NOs: 4 and 46, respectively;
b) SEQ ID NOs: 28 and 8, respectively;
c) SEQ ID NOs: 22 and 8, respectively;
d) SEQ ID NOs: 4 and 8, respectively;
e) SEQ ID NOs: 16 and 8, respectively;
f) SEQ ID NOs: 4 and 34, respectively;
g) SEQ ID NOs: 4 and 38, respectively;
h) SEQ ID NOs: 4 and 42, respectively;
i) SEQ ID NOs: 4 and 50, respectively;
j) SEQ ID NOs: 4 and 54, respectively;
k) SEQ ID NOs: 16 and 34, respectively;
l) SEQ ID NOs: 16 and 38, respectively;
m) SEQ ID NOs: 16 and 42, respectively;
n) SEQ ID NOs: 16 and 46, respectively;
o) SEQ ID NOs: 16 and 50, respectively;
p) SEQ ID NOs: 16 and 54, respectively;
q) SEQ ID NOs: 22 and 38, respectively;
r) SEQ ID NOs: 22 and 42, respectively;
s) SEQ ID NOs: 22 and 46, respectively;
t) SEQ ID NOs: 22 and 50, respectively;
u) SEQ ID NOs: 28 and 38, respectively;
v) SEQ ID NOs: 28 and 42, respectively;
w) SEQ ID NOs: 28 and 46, respectively;
x) SEQ ID NOs: 28 and 50, respectively;
y) SEQ ID NOs: 28 and 54, respectively; or
z) SEQ ID NOs: 70 and 71, respectively.

In certain embodiments, the anti-TNFα antibody comprises an HC comprising a sequence selected from SEQ ID NOs: 2, 14, 20, and 26 and an LC comprising a sequence selected from SEQ ID NOs: 6, 32, 36, 40, 44, 48, and 52. In particular embodiments, the anti-TNFα antibody comprises an HC and an LC that comprise:
a) SEQ ID NOs: 2 and 44, respectively;
b) SEQ ID NOs: 26 and 6, respectively;
c) SEQ ID NOs: 20 and 6, respectively;
d) SEQ ID NOs: 14 and 6, respectively;
e) SEQ ID NOs: 2 and 32, respectively;
f) SEQ ID NOs: 2 and 36, respectively;
g) SEQ ID NOs: 2 and 40, respectively;
h) SEQ ID NOs: 2 and 48, respectively;
i) SEQ ID NOs: 2 and 52, respectively;
j) SEQ ID NOs: 14 and 32, respectively;
k) SEQ ID NOs: 14 and 36, respectively;
l) SEQ ID NOs: 14 and 40, respectively;
m) SEQ ID NOs: 14 and 44, respectively;
n) SEQ ID NOs: 14 and 48, respectively;
o) SEQ ID NOs: 14 and 52, respectively;
p) SEQ ID NOs: 20 and 36, respectively;
q) SEQ ID NOs: 20 and 40, respectively;
r) SEQ ID NOs: 20 and 44, respectively;
s) SEQ ID NOs: 20 and 48, respectively;
t) SEQ ID NOs: 26 and 36, respectively;
u) SEQ ID NOs: 26 and 40, respectively;
v) SEQ ID NOs: 26 and 44, respectively;
w) SEQ ID NOs: 26 and 48, respectively; or
x) SEQ ID NOs: 26 and 52.

An anti-TNFα antibody described herein may be monovalent/in a monomeric format. Examples of such formats include any format comprising a single antigen-binding domain (e.g., a single VH/VL pair), including Fab, scFv, single domain antibody, VHH/nanobody, UniDab, VNAR etc. Also contemplated are monovalent forms of binding molecules such as adnexins, affibodies, affilins, anticalins, avimers, and DARPins, wherein the binding molecules have the binding specificity of an anti-TNFα antibody described herein. A monovalent antibody described herein may comprise a constant (Fc) region component (e.g., a full Fc region) that provides effector function (e.g., full effector function).

In certain embodiments, a monovalent anti-TNFα antibody described herein comprises an antigen-binding protein, which may be monovalent, bivalent, or multivalent. In some embodiments, the antigen-binding protein is monovalent (also termed a “Fab” herein) and comprises a VH and a VL, or an HC and an LC, of an anti-TNFα antibody described herein. In particular embodiments, the antigen-binding protein is monovalent and comprises an HC and an LC of an anti-TNFα antibody described herein. In some embodiments, a monovalent anti-TNFα antibody described herein is a heterotrimer comprising an antibody HC coupled to an antibody LC to form an antigen-binding domain, wherein the antibody HC dimerizes with a polypeptide that is a “truncated heavy chain” (i.e., an HC lacking the variable and CH1 domains) to form an Fc domain. The truncated heavy chain may comprise or consist of an Fc monomer (i.e., one of two polypeptides that dimerize to form an Fc domain). In certain embodiments, the Fc monomer comprises CH2 and CH3 of an antibody heavy chain such as an IgG heavy chain; the IgG may be IgG1, IgG2, IgG2, or IgG4. In particular embodiments, dimerization between the antibody HC and the truncated HC provides a fully functional Fc domain, which may preserve the pharmacokinetic and effector function properties of the parent antibody (e.g., Ab1).

In certain embodiments, a monovalent anti-TNFα antibody described herein comprises an scFv. In certain embodiments, the scFv comprises a VH and a VL of an anti-TNFα antibody described herein. In particular embodiments, the monovalent anti-TNFα antibody described herein is a heterodimer (e.g., a single chain comprising an scFv and Fc monomer of an anti-TNFα antibody described herein, and an additional (truncated) HC lacking the variable domain and CH1 domain (e.g., a constant domain fragment such as an Fc monomer). The single chain may be arranged, for example, as VL-linker-VH-Fc monomer. In particular embodiments, dimerization between the Fc monomer portion of the single chain and the Fc monomer portion of the additional HC provides a fully functional Fc domain, which may preserve the pharmacokinetic and effector function properties of the parent antibody (e.g., Ab1).

In certain embodiments of the monovalent anti-TNFα antibody heterotrimer or heterodimer, the heavy chain Fc heterodimer is, e.g., in a format described in Brinkmann and Kontermann, MAbs 9:182-212 (2017). For example, a “knobs-into-holes,” HA-TF, ZW1, CH3 charge pair, EW-RVT, LUZ-Y, Strand Exchange Engineered Domain body (SEEDbody), Biclonic, DuoBody, BEAT, 7.8.60, 20.8.34, Triomab/Quadroma, or CrossMAb strategy may be used to promote heterodimerization (e.g., over homodimerization) of the antibody heavy chain Fc monomer and the truncated heavy chain Fc monomer. In certain embodiments, a “knobs-into-holes” approach may be used, wherein a “knob” variant of a domain is obtained by replacing an amino acid with a small side chain (for example, alanine, asparagine, aspartic acid, glycine, serine, threonine or valine) with another amino acid with a larger side chain (for example, arginine, phenylalanine, tyrosine, or tryptophan). A “hole” variant of a domain is obtained by replacing an amino acid with a large side chain (for example, arginine, phenylalanine, tyrosine, or tryptophan) with another amino acid with a smaller side chain (for example, alanine, asparagine, aspartic acid, glycine, serine, threonine or valine). In certain embodiments, the knob and/or hole mutations are in the CH3 domain. In particular embodiments, both Fc monomers are derived from IgG1, and the antibody heavy chain Fc monomer may comprise mutations T366S, L368A, and Y407A in the CH3 domain and the truncated heavy chain Fc monomer may comprise the mutation T366W in the CH3 domain, or vice-versa, wherein the residues are numbered according to the Eu system. Additionally or alternatively, the antibody heavy chain Fc monomer may comprise the mutation Y349C and the truncated heavy chain Fc monomer may comprise the mutation S354C, or vice-versa, wherein the residues are numbered according to the Eu system.

In certain embodiments, a monovalent anti-TNFα antibody of the present disclosure comprises an antigen-binding protein HC comprising a sequence selected from 10, 18, 24, and 30; an antigen-binding protein LC comprising a sequence selected from 6, 32, 36, 40, 44, 48, and 52; and a truncated HC comprising SEQ ID NO: 12. In particular embodiments, the antigen-binding protein HC, antigen-binding protein LC, and truncated HC comprise:
a) SEQ ID NOs: 10, 44, and 12, respectively;
b) SEQ ID NOs: 30, 6, and 12, respectively;
c) SEQ ID NOs: 24, 6, and 12, respectively;
d) SEQ ID NOs: 10, 6, and 12, respectively;
e) SEQ ID NOs: 18, 6, and 12, respectively;
f) SEQ ID NOs: 10, 32, and 12, respectively;
g) SEQ ID NOs: 10, 36, and 12, respectively;
h) SEQ ID NOs: 10, 40, and 12, respectively;
i) SEQ ID NOs: 10, 48, and 12, respectively;
j) SEQ ID NOs: 10, 52, and 12, respectively;
k) SEQ ID NOs: 18, 32, and 12, respectively;
l) SEQ ID NOs: 18, 36, and 12, respectively;
m) SEQ ID NOs: 18, 40, and 12, respectively;
n) SEQ ID NOs: 18, 44, and 12, respectively;
o) SEQ ID NOs: 18, 48, and 12, respectively;
p) SEQ ID NOs: 18, 52, and 12, respectively;
q) SEQ ID NOs: 24, 36, and 12, respectively;
r) SEQ ID NOs: 24, 40, and 12, respectively;
s) SEQ ID NOs: 24, 44, and 12, respectively;
t) SEQ ID NOs: 24, 48, and 12, respectively;
u) SEQ ID NOs: 30, 36, and 12, respectively;
v) SEQ ID NOs: 30, 40, and 12, respectively;
w) SEQ ID NOs: 30, 44, and 12, respectively;
x) SEQ ID NOs: 30, 48, and 12, respectively; or
y) SEQ ID NOs: 30, 52, and 12, respectively.

The present disclosure also provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 55, 56, 57, 58, 59, and 68, respectively. In some embodiments, the antibody or portion comprises a VH comprising SEQ ID NO: 4 and a VL comprising SEQ ID NO: 46. In some embodiments, the antibody comprises an HC comprising SEQ ID NO: 2 and an LC comprising SEQ ID NO: 44. In certain embodiments, the antibody is monovalent and comprises a monovalent antigen-binding protein and a truncated HC lacking the variable domain and CH1 domain, wherein said antigen-binding protein has an HC that comprises SEQ ID NO: 10 and an LC that comprises SEQ ID NO: 44 and said truncated HC comprises SEQ ID NO: 12, wherein the antigen-binding protein HC and the truncated HC are capable of dimerization.

The present disclosure also provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 55, 56, 63, 58, 59, and 60, respectively. In some embodiments, the antibody or portion comprises a VH comprising SEQ ID NO: 28 and a VL comprising SEQ ID NO: 8. In some embodiments, the antibody comprises an HC comprising SEQ ID NO: 26 and an LC comprising SEQ ID NO: 6. In certain embodiments, the antibody is monovalent and comprises a monovalent antigen-binding protein and a truncated HC lacking the variable domain and CH1 domain, wherein said antigen-binding protein has an HC that comprises SEQ ID NO: 30 and an LC that comprises SEQ ID NO: 6 and said truncated HC comprises SEQ ID NO: 12, wherein the antigen-binding protein HC and the truncated HC are capable of dimerization.

The present disclosure also provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 55, 56, 62, 58, 59, and 60, respectively. In some embodiments, the antibody or portion comprises a VH comprising SEQ ID NO: 22 and a VL comprising SEQ ID NO: 8. In some embodiments, the antibody comprises an HC comprising SEQ ID NO: 20 and an LC comprising SEQ ID NO: 6. In certain embodiments, the antibody is monovalent and comprises a monovalent antigen-binding protein and a truncated HC lacking the variable domain and CH1 domain, wherein said antigen-binding protein has an HC that comprises SEQ ID NO: 24 and an LC that comprises SEQ ID NO: 6 and said truncated HC comprises SEQ ID NO: 12, wherein the antigen-binding protein HC and the truncated HC are capable of dimerization.

In some embodiments, the constant region(s) of an anti-TNFα antibody or antigen-binding portion thereof described herein are mutated, e.g., to increase the effector function of the antibody or antigen-binding portion (e.g., as described in Wang et al., Protein Cell (2018) 9(1):63-73; Kellner et al., Transfus Med Hemother. (2017) 44:327-36; or Robkopf et al., Antibodies (2020) 9 (4): 63). In certain embodiments, the mutations enhance ADCC or CDC. In some embodiments, the mutations are in an IgG1 and comprise (Eu numbering) L235V, G236A, S239D, F243L, S267E, H268F, R292P, S298A, Y300L, V305I, S324T, N325S, K326W, L328F, A330L, 1332E, E333A, E333S, K334A, P396L, or any combination thereof. For example, the mutations may comprise F243L/R292P/Y300L/V305I/P396L; S239D/1332E; S239D/1332E/A330L; S298A/E333A/K334A; L235V/F143L/R292P/Y300L/P396L; G236A/S239D/1332E; K326W/E333S; S267E/H268F/S324T; S267E/H268F/S324T/G236A/1332E; S267E/L328F; or N325S/L328F. In some embodiments, the mutations may comprise L234Y/L235Q/G236W/S239M/H268D/D270E/S298A on one heavy chain and D270E/K326D/A330M/K334E on the other heavy chain.

Additionally or alternatively, the constant region(s) of an anti-TNFα antibody or antigen-binding portion thereof described herein may be mutated to prolong the half-life of the antibody or portion (e.g., as described in Maeda et al., MAbs (2017) 9 (5): 844-53; Wang et al., supra; or PCT Patent Publication WO 00/09560). In some embodiments, the mutations are in an IgG1 and comprise (Eu numbering) M252Y, S254T, T256E, M428L N434A, N434S, Y436T, Y436V, Q438R, S440E, or any combination thereof. For example, the mutations may comprise M252Y/S254T/T256E, M428L/N434S, N434A/Y436T/Q438R/S440E; N434A/Y436V/Q438R/S440E; M428L/N434A/Y436T/Q438R/S440E; M428L/N434A/Y436V/Q438R/S440E; or M428L/N434A/Q438R/S440E.

In some embodiments, the antibody is glycoengineered to enhance effector function (e.g., as described in Li et al., Proc Natl Acad Sci USA (2017) 114(13):3485-90; or Robkopf et al., supra). In certain embodiments, the antibody is glycoengineered to reduce fucose (e.g., afucosylated variants) or sialic acid content or through GlycoMAb™ technology.

In some embodiments, the framework or constant region(s) of an anti-TNFα antibody or antigen-binding portion thereof described herein are mutated to alter the immunogenicity of the antibody, and/or to provide a site for covalent or non-covalent binding to another molecule.

Any combination of the mutations described herein is also contemplated.

In some embodiments, an anti-TNFα antibody or antigen-binding portion of the present disclosure binds to human TNFα with an EC50 of no more than 1e-007 M, 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M, e.g., at pH 7.4. In certain embodiments, binding of the antibody or antigen-binding portion to human TNFα is reduced by at least 2-, 5-, 10-, 15-, 20-, 25-, 30-, 100-, 500-, 1000-, 1500-, 2000-, 2500-, 3000-, or 4000-fold at pH 6.0. In particular embodiments, the antibody or antigen-binding portion has a dissociation rate at pH 6.0 that is at least 20-, 30-, 40-, 50-, 75-, 100-, 150-, 200-, 250-, 300-, 400-, 500-, 600-, 700-, 800-, 900-, 1000-, 1500-, 2000-, or 2500-fold faster than that of Ab1 or monovalent Ab1. In some embodiments, the antibody or antigen-binding portion binds to human TNFα with an EC50 of no more than 50 nM at pH 7.4 and has a dissociation rate for human TNFα at pH 6.0 that is at least 10-fold, 100-fold, or 1000-fold greater than the dissociation rate of Ab1 or monovalent Ab1. In some embodiments, the antibody or antigen-binding portion binds to human TNFα with an EC50 of no more than 50 or 100 nM at pH 7.4 and has a dissociation rate for human TNFα of greater than 2e-004 s⁻¹ at pH 6.0.

In some embodiments, an anti-TNFα antibody or antigen-binding portion of the present disclosure binds to cynomolgus TNFα with an EC50 of no more than 1e-007 M, 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M, e.g., at pH 7.4. In certain embodiments, binding of the antibody or antigen-binding portion to cynomolgus TNFα is reduced by at least 2-, 5-, 10-, 15-, 20-, 25-, 30-, 100-, 500-, 1000-, 1500-, 2000-, 2500-, 3000-, or 4000-fold at pH 6.0. In particular embodiments, the antibody or antigen-binding portion has a dissociation rate at pH 6.0 that is at least 20-, 30-, 40-, 50-, 75-, 100-, 150-, 200-, 250-, 300-, 400-, 500-, 600-, 700-, 800-, 900-, 1000-, 1500-, 2000-, or 2500-fold faster than that of Ab1. In some embodiments, the antibody or antigen-binding portion binds to cynomolgus TNFα with an EC50 of no more than 50 nM at pH 7.4 and has a dissociation rate for cynomolgus TNFα at pH 6.0 that is at least 10-fold, 100-fold, or 1000-fold greater than the dissociation rate of Ab1 or monovalent Ab1. In some embodiments, the antibody or antigen-binding portion binds to cynomolgus TNFα with an EC50 of no more than 50 or 100 nM at pH 7.4 and has a dissociation rate for cynomolgus TNFα of greater than 2e-004 s⁻¹ at pH 6.0.

In some embodiments, an anti-TNFα antibody or antigen-binding portion of the present disclosure binds to murine TNFα with an EC50 of no more than 1e-006 M, 5e-007 M, 1e-007 M, 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M, e.g., at pH 7.4. In certain embodiments, binding of the antibody or antigen-binding portion to cynomolgus TNFα is reduced by at least 2-, 5-, 10-, 15-, 20-, 25-, 30-, 100-, 500-, 1000-, 1500-, 2000-, 2500-, 3000-, or 4000-fold at pH 6.0. In particular embodiments, the antibody or antigen-binding portion has a dissociation rate at pH 6.0 that is at least 20-, 30-, 40-, 50-, 75-, 100-, 150-, 200-, 250-, 300-, 400-, 500-, 600-, 700-, 800-, 900-, 1000-, 1500-, 2000-, or 2500-fold faster than that of Ab1.

In certain embodiments, an anti-TNFα antibody or antigen-binding portion of the present disclosure binds to human, cynomolgus, and murine TNFα, for example with an EC50 of no more than 1e-008 M, 5e-009 M, 2e-009M, 1e-009M, 5e-010 M, 2e-010 M, 1e-011 M, 5e-011 M, 2e-011 M, 1e-011 M, 5e-012 M, 2e-012 M, or 1e-012 M, or any combination thereof, for each antigen, e.g., at pH 7.4.

In some embodiments, an anti-TNFα antibody or antigen-binding portion of the present disclosure binds to human membrane-associated TNFα (mTNFα) with an EC50 of no more than 5e-008 M, 2e-008 M, 1e-008 M, 5e-009 M, 2e-009 M, 1e-009 M, 5e-010 M, 2e-010 M, 1e-011 M, 5e-011 M, 2e-011 M, or 1e-011 M.

In some embodiments, an anti-TNFα antibody or antigen-binding portion of the present disclosure inhibits TNFα stimulation of monocytes. In certain embodiments, the antibody or antigen-binding portion inhibits human or cynomolgus TNFα stimulation of HEK-Blue™ TNFα cells with an IC50 of no more than 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M. In certain embodiments, the antibody or antigen-binding portion inhibits human TNFα stimulation of THP1-Blue™ NF-κB cells with an IC50 of no more than 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M.

In some embodiments, an anti-TNFα antibody or antigen-binding portion of the present disclosure does not form large IC (i.e., two or more TNFα molecules cross-linked by three or more antibody molecules).

In some embodiments, an anti-TNFα antibody or antigen-binding portion of the present disclosure undergoes less degradation, and/or undergoes increased recycling to the cell surface, in vivo in comparison to Ab1.

In some embodiments, an anti-TNFα antibody or antigen-binding portion of the present disclosure is less immunogenic in vivo than Ab1. The antibody or antigen-binding portion may produce less of an anti-drug antibody (ADA) response in vivo. For example, in certain embodiments, the antibody or antigen-binding portion results in less formation of IgG and/or IgM anti-drug antibodies in mice at 7, 14, or 21 days after administration of the antibody or antigen-binding portion.

In some embodiments, an anti-TNFα antibody or antigen-binding portion of the present disclosure has a longer half-life in vivo than Ab1. In certain embodiments, the half-life may be at least 2, 3, 4, 5, 6, 7, 8, 9, 10, 20, 40, 60, 80, or 100 times longer than the half-life of Ab1.

The present disclosure also contemplates an anti-TNFα antibody or antigen-binding portion with any combination of the above properties.

In some embodiments, an anti-TNFα antibody or antigen-binding portion of the present disclosure has one, two, three, four, five, six, or seven properties selected from:
competes for binding with, or binds to the same epitope of human TNFα as, a reference antibody comprising heavy and light chain amino acid sequences of:
SEQ ID NOs: 1 and 6;
SEQ ID NOs: 72 and 73;
SEQ ID NOs: 74 and 75; or
SEQ ID NOs: 76 and 77;
binds to human TNFα with an affinity similar to that of a reference antibody comprising heavy and light chain amino acid sequences of:
SEQ ID NOs: 1 and 6;
SEQ ID NOs: 72 and 73;
SEQ ID NOs: 74 and 75; or
SEQ ID NOs: 76 and 77;
inhibits TNFα stimulation of monocytes;
does not form large immune complexes (e.g., with two or more TNFα molecules crosslinked by three or more antibodies);
binds membrane-associated TNFα;
is less immunogenic in vivo than a reference antibody comprising heavy and light chain amino acid sequences of:
SEQ ID NOs: 1 and 6;
SEQ ID NOs: 72 and 73;
SEQ ID NOs: 74 and 75; or
SEQ ID NOs: 76 and 77; and
has a longer half-life in vivo than a reference antibody comprising heavy and light chain amino acid sequences of:
SEQ ID NOs: 1 and 6;
SEQ ID NOs: 72 and 73;
SEQ ID NOs: 74 and 75; or
SEQ ID NOs: 76 and 77.

In certain embodiments, the reference antibody has heavy and light chain amino acid sequence of SEQ ID NOs: 1 and 6. In certain embodiments, the anti-TNFα antibody or antigen-binding portion has all of said properties (e.g., wherein the reference antibody has heavy and light chain amino acid sequence of SEQ ID NOs: 1 and 6).

An anti-TNFα antibody or antigen-binding portion of the present disclosure can be derivatized or linked to another molecule (e.g., another peptide or protein). In general, the antibodies or portions thereof are derivatized such that TNFα binding is not affected adversely by the derivatization or labeling. Accordingly, the antibodies and antibody portions of the present disclosure are intended to include both intact and modified forms of the anti-TNFα antibodies and portions described herein. For example, an antibody or antibody portion of the present disclosure can be functionally linked (by chemical coupling, genetic fusion, noncovalent association or otherwise) to one or more other molecular entities, such as another antibody (e.g., to form a bispecific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m maleimidobenzoyl-N-hydroxysuccinimide ester) or homobifunctional (e.g., disuccinimidyl suberate). Such linkers are available, e.g., from Pierce Chemical Company, Rockford, IL.

An anti-TNFα antibody or antigen-binding portion thereof can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody or antigen-binding portion according to the present disclosure may also be labeled. As used herein, the terms “label” or “labeled” refer to incorporation of another molecule in the antibody. In some embodiments, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In some embodiments, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies of the present disclosure may be present in a neutral form (including zwitterionic forms) or as a positively or negatively-charged species. In some embodiments, the antibodies may be complexed with a counterion to form a pharmaceutically acceptable salt.

### Bispecific Binding Molecules

In a further aspect, the present disclosure provides a bispecific binding molecule having the binding specificity (e.g., comprising the antigen-binding portion, such as the six CDRs or the VH and VL) of an anti-TNFα antibody described herein and the binding specificity of a second, distinct antibody. The second antibody may be, e.g., another anti-TNFα antibody (e.g., another antibody described herein), or an antibody that targets a different protein, such as another cell surface molecule whose activity mediates an autoimmune or inflammatory condition. In certain embodiments, the second antibody targets IL17A, IL23, or angiopoietin 2.

The present disclosure also contemplates multispecific antibodies having the binding specificity of an anti-TNFα antibody described herein and the binding specificity of more than one additional antibody (e.g., two or three additional antibodies).

In certain embodiments, a bispecific binding molecule described herein is used in place of an anti-TNFα antibody or antigen-binding portion described herein in any aspect of the present disclosure (e.g., a therapeutic method, article of manufacture, or kit as described herein).

### Immunoconjugates

In a further aspect, the present disclosure provides an immunoconjugate comprising an anti-TNFα antibody or antigen-binding portion described herein conjugated to a therapeutic agent. In some embodiments, the therapeutic agent is an anti-inflammatory or immunosuppressive agent. In certain embodiments, the therapeutic agent is a steroid, such as a glucocorticoid receptor modulator (e.g., agonist). For example, the therapeutic agent may be selected from dexamethasone, prednisolone, budesonide, and the like. In some embodiments, the therapeutic agent may be any payload described in PCT Patent Application WO 2021/161263 or WO 2017/210471, both of which are incorporated by reference in their entirety herein.

In particular embodiments, the therapeutic agent may have the structure of Formula I below.

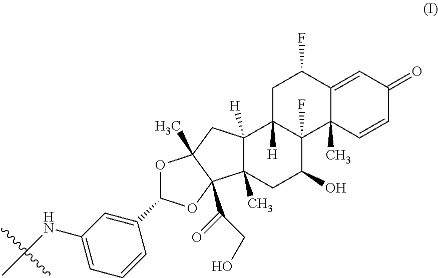

In particular embodiments, the therapeutic agent may have the structure of Formula II below.

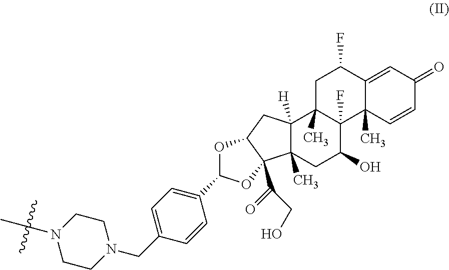

In certain embodiments, an immunoconjugate described herein is used in place of an anti-TNFα antibody or antigen-binding portion described herein in any aspect of the present disclosure (e.g., a therapeutic method, article of manufacture, or kit as described herein).

### Nucleic Acid Molecules and Vectors

The present disclosure also provides nucleic acid molecules and sequences encoding anti-TNFα antibodies or antigen-binding portions described herein. In some embodiments, different nucleic acid molecules encode the heavy chain and light chain amino acid sequences of the anti-TNFα antibody or antigen-binding portion. In other embodiments, the same nucleic acid molecule encodes the heavy chain and light chain amino acid sequences of the anti-TNFα antibody or antigen-binding portion. The present disclosure thus provides an isolated nucleic acid molecule comprising a nucleotide sequence that encodes a heavy chain or an antigen-binding portion thereof, or a nucleotide sequence that encodes a light chain or an antigen-binding portion thereof, or both, of an anti-TNFα antibody or antigen-binding portion described herein.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single- and double-stranded forms.

In any of the above embodiments, the nucleic acid molecules may be isolated. Nucleic acid molecules referred to herein as “isolated” or “purified” are nucleic acids which (1) have been separated away from the nucleic acids of the genomic DNA or cellular RNA of their source of origin; and/or (2) do not occur in nature.

In some embodiments, nucleic acid molecule(s) of the present disclosure comprise nucleotide sequences that encode H-CDR1-3 and/or L-CDR1-3 of an anti-TNFα antibody or antigen-binding portion of the present disclosure. In some embodiments, nucleic acid molecule(s) of the present disclosure comprise nucleotide sequences that encode the VH and/or VL of an anti-TNFα antibody or antigen-binding portion of the present disclosure. In some embodiments, nucleic acid molecule(s) of the present disclosure comprises nucleotide sequences that encode the HC(s) and/or LC of an anti-TNFα antibody or antigen-binding portion of the present disclosure.

In some embodiments, a nucleic acid molecule of the present disclosure comprises one or more nucleotide sequences selected from the group consisting of SEQ ID NOs: 1, 3, 5, 7, 9, 11, 13, 15, 17, 19, 21, 23, 24, 27, 29, 31, 33, 35, 37, 39, 41, 43, 45, 47, 49, 51, and 53.

In certain embodiments, nucleic acid molecule(s) of the present disclosure comprise the nucleotide sequences of:
a) SEQ ID NOs: 3 and 45;
b) SEQ ID NOs: 27 and 7;
c) SEQ ID NOs: 21 and 7;
d) SEQ ID NOs: 3 and 7;
e) SEQ ID NOs: 15 and 7;
f) SEQ ID NOs: 3 and 33;
g) SEQ ID NOs: 3 and 37;
h) SEQ ID NOs: 3 and 41;
i) SEQ ID NOs: 3 and 49;
j) SEQ ID NOs: 3 and 53;
k) SEQ ID NOs: 15 and 33;
l) SEQ ID NOs: 15 and 37;
m) SEQ ID NOs: 15 and 41;
n) SEQ ID NOs: 15 and 45;
o) SEQ ID NOs: 15 and 49;
p) SEQ ID NOs: 15 and 53;
q) SEQ ID NOs: 21 and 37;
r) SEQ ID NOs: 21 and 41;
s) SEQ ID NOs: 21 and 45;
t) SEQ ID NOs: 21 and 49;
u) SEQ ID NOs: 27 and 37;
v) SEQ ID NOs: 27 and 41;
w) SEQ ID NOs: 27 and 45;
x) SEQ ID NOs: 27 and 49; or
y) SEQ ID NOs: 27 and 53.

In certain embodiments, nucleic acid molecule(s) of the present disclosure comprise the nucleotide sequences of:
a) SEQ ID NOs: 9, 43, and 11;
b) SEQ ID NOs: 29, 5, and 11;
c) SEQ ID NOs: 23, 5, and 11;
d) SEQ ID NOs: 9, 5, and 11;
e) SEQ ID NOs: 17, 5, and 11;
f) SEQ ID NOs: 9, 31, and 11;
g) SEQ ID NOs: 9, 35, and 11;
h) SEQ ID NOs: 9, 39, and 11;
i) SEQ ID NOs: 9, 47, and 11;
j) SEQ ID NOs: 9, 51, and 11;
k) SEQ ID NOs: 17, 31, and 11;
l) SEQ ID NOs: 17, 35, and 11;
m) SEQ ID NOs: 17, 39, and 11;
n) SEQ ID NOs: 17, 43, and 11;
o) SEQ ID NOs: 17, 47, and 11;
p) SEQ ID NOs: 17, 51, and 11;
q) SEQ ID NOs: 23, 35, and 11;
r) SEQ ID NOs: 23, 39, and 11;
s) SEQ ID NOs: 23, 43, and 11;
t) SEQ ID NOs: 23, 47, and 11;
u) SEQ ID NOs: 29, 35, and 11;
v) SEQ ID NOs: 29, 39, and 11;
w) SEQ ID NOs: 29, 43, and 11;
x) SEQ ID NOs: 29, 47, and 11; or
y) SEQ ID NOs: 29, 51, and 11.

In certain embodiments, nucleic acid molecule(s) of the present disclosure comprise the nucleotide sequences of:
a) SEQ ID NOs: 1 and 43;
b) SEQ ID NOs: 15 and 5;
c) SEQ ID NOs: 9 and 5;
d) SEQ ID NOs: 13 and 5;
e) SEQ ID NOs: 1 and 31;
f) SEQ ID NOs: 1 and 35;
g) SEQ ID NOs: 1 and 39;
h) SEQ ID NOs: 1 and 47;
i) SEQ ID NOs: 1 and 51;
j) SEQ ID NOs: 13 and 31;
k) SEQ ID NOs: 13 and 35;
l) SEQ ID NOs: 13 and 39;
m) SEQ ID NOs: 13 and 43;
n) SEQ ID NOs: 13 and 47;
o) SEQ ID NOs: 13 and 51;
p) SEQ ID NOs: 9 and 35;
q) SEQ ID NOs: 9 and 39;
r) SEQ ID NOs: 9 and 43;
s) SEQ ID NOs: 9 and 47;
t) SEQ ID NOs: 15 and 35;
u) SEQ ID NOs: 15 and 39;
v) SEQ ID NOs: 15 and 43;
w) SEQ ID NOs: 15 and 47; or
x) SEQ ID NOs: 15 and 51.

In any of the above embodiments of nucleic acid molecule(s), the nucleotide sequences may be on the same nucleic acid molecule, or on a set of nucleic acid molecules.

The present disclosure further provides a vector comprising nucleic acid molecules that encode the heavy chain(s) and light chain of an anti-TNFα antibody as described herein or an antigen-binding portion thereof. In certain embodiments, a vector of the present disclosure comprises nucleic acid molecule(s) as described herein. The vector may further comprise an expression control sequence.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

In some embodiments of the nucleic acid molecule(s) described herein, the nucleotide sequences may be arranged as two coding sequences (e.g., for a heterodimeric monovalent antibody described herein, a first coding sequence encoding the VH, VL, CH1, and Fc monomer regions, and a second coding sequence encoding a truncated HC) or three coding sequences (e.g., for a heterotrimeric monovalent antibody described herein, first and second coding sequences encoding antigen-binding protein HC and LC sequences, respectively, and a third coding sequence encoding an additional truncated HC). In certain embodiments, the coding sequences are in a polycistronic arrangement on a single nucleic acid molecule. The coding sequences of a polycistronic construct can be separated from each other, e.g., by the coding sequence of a self-cleaving peptide, or can be separated by a ribosomal internal entry site (IRES). Thus, the polycistronic construct may be transcribed as a single RNA that is processed and translated as separate polypeptides. In other embodiments, the coding sequences are on two or three separate nucleic acid molecules (e.g., for heterodimeric and heterotrimeric antibodies, respectively). The coding sequences may be under the control of the same or different promoters.

### Host Cells and Methods of Antibody Production

The present disclosure also provides methods for producing the antibodies and antigen-binding portions thereof described herein. In some embodiments, the present disclosure provides a host cell comprising nucleotide sequences that encode the heavy chain(s) and the light chain of an anti-TNFα antibody or antigen-binding portion described herein, wherein the nucleotide sequences may be on the same or different nucleic acid molecules. In some embodiments, the host cell comprises one or more vectors as described herein. In some embodiments, the present disclosure relates to a method for producing an anti-TNFα antibody or antigen-binding portion as described herein, comprising providing said host cell; culturing said host cell under conditions suitable for expression of the antibody or antigen-binding portion; and isolating the resulting antibody or antigen-binding portion. Antibodies or antigen-binding portions produced by such expression in such recombinant host cells are referred to herein as “recombinant” antibodies or antigen-binding portions. The present disclosure also provides progeny cells of such host cells, and antibodies or antigen-binding portions produced by same.

The term “recombinant host cell” (or simply “host cell”), as used herein, means a cell into which a recombinant expression vector has been introduced. By definition, a recombinant host cell does not occur in nature. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-TNFα antibodies and antigen-binding portions thereof described herein, and vectors comprising these nucleic acid molecules, can be used for transfection of a suitable mammalian, plant, bacterial or yeast host cell. In some embodiments, the nucleotide sequence encoding the light chain is transfected into the cell at a ratio of, e.g., 4:1, 2:1, or 1:1 relative to the nucleotide sequence encoding the heavy chain. In some embodiments, where the TNFα antibody has a heterotrimeric architecture as described herein, the nucleotide sequences encoding the antibody light chain, the “knob” heavy chain (e.g., the truncated heavy chain), and the “hole” heavy chain (e.g., the antibody heavy chain) may be transfected at a ratio of, e.g., 4:2:1 or 6:2:1. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

It is likely that antibodies expressed by different cell lines or in transgenic animals will have different glycosylation patterns from each other. However, all antibodies encoded by the nucleic acid molecules provided herein, or comprising the amino acid sequences provided herein are part of the present disclosure, regardless of the glycosylation state of the antibodies, and more generally, regardless of the presence or absence of post-translational modification(s).

In some embodiments, a host cell of the present disclosure comprises nucleotide sequences that encode H-CDR1-3 and/or L-CDR1-3, VH and/or VL, or HC(s) and/or LC of an anti-TNFα antibody or antigen-binding portion of the present disclosure.

In some embodiments, a host cell of the present disclosure comprises one or more nucleotide sequences selected from the group consisting of SEQ ID NOs: 1, 3, 5, 7, 9, 11, 13, 15, 17, 19, 21, 23, 24, 27, 29, 31, 33, 35, 37, 39, 41, 43, 45, 47, 49, 51, and 53.

In certain embodiments, a host cell of the present disclosure comprises the nucleotide sequences of:
a) SEQ ID NOs: 3 and 45;
b) SEQ ID NOs: 27 and 7;
c) SEQ ID NOs: 21 and 7;
d) SEQ ID NOs: 3 and 7;
e) SEQ ID NOs: 15 and 7;
f) SEQ ID NOs: 3 and 33;
g) SEQ ID NOs: 3 and 37;
h) SEQ ID NOs: 3 and 41;
i) SEQ ID NOs: 3 and 49;
j) SEQ ID NOs: 3 and 53;
k) SEQ ID NOs: 15 and 33;
l) SEQ ID NOs: 15 and 37;
m) SEQ ID NOs: 15 and 41;
n) SEQ ID NOs: 15 and 45;
o) SEQ ID NOs: 15 and 49;
p) SEQ ID NOs: 15 and 53;
q) SEQ ID NOs: 21 and 37;
r) SEQ ID NOs: 21 and 41;
s) SEQ ID NOs: 21 and 45;
t) SEQ ID NOs: 21 and 49;
u) SEQ ID NOs: 27 and 37;
v) SEQ ID NOs: 27 and 41;
w) SEQ ID NOs: 27 and 45;
x) SEQ ID NOs: 27 and 49; or
y) SEQ ID NOs: 27 and 53.

In certain embodiments, a host cell of the present disclosure comprises the nucleotide sequences of:
a) SEQ ID NOs: 9, 43, and 11;
b) SEQ ID NOs: 29, 5, and 11;
c) SEQ ID NOs: 23, 5, and 11;
d) SEQ ID NOs: 9, 5, and 11;
e) SEQ ID NOs: 17, 5, and 11;
f) SEQ ID NOs: 9, 31, and 11;
g) SEQ ID NOs: 9, 35, and 11;
h) SEQ ID NOs: 9, 39, and 11;
i) SEQ ID NOs: 9, 47, and 11;
j) SEQ ID NOs: 9, 51, and 11;
k) SEQ ID NOs: 17, 31, and 11;
l) SEQ ID NOs: 17, 35, and 11;
m) SEQ ID NOs: 17, 39, and 11;
n) SEQ ID NOs: 17, 43, and 11;
o) SEQ ID NOs: 17, 47, and 11;
p) SEQ ID NOs: 17, 51, and 11;
q) SEQ ID NOs: 23, 35, and 11;
r) SEQ ID NOs: 23, 39, and 11;
s) SEQ ID NOs: 23, 43, and 11;
t) SEQ ID NOs: 23, 47, and 11;
u) SEQ ID NOs: 29, 35, and 11;
v) SEQ ID NOs: 29, 39, and 11;
w) SEQ ID NOs: 29, 43, and 11;
x) SEQ ID NOs: 29, 47, and 11; or
y) SEQ ID NOs: 29, 51, and 11.

In certain embodiments, a host cell of the present disclosure comprises the nucleotide sequences of:
a) SEQ ID NOs: 1 and 43;
b) SEQ ID NOs: 15 and 5;
c) SEQ ID NOs: 9 and 5;
d) SEQ ID NOs: 13 and 5;
e) SEQ ID NOs: 1 and 31;
f) SEQ ID NOs: 1 and 35;
g) SEQ ID NOs: 1 and 39;
h) SEQ ID NOs: 1 and 47;
i) SEQ ID NOs: 1 and 51;
j) SEQ ID NOs: 13 and 31;
k) SEQ ID NOs: 13 and 35;
l) SEQ ID NOs: 13 and 39;
m) SEQ ID NOs: 13 and 43;
n) SEQ ID NOs: 13 and 47;
o) SEQ ID NOs: 13 and 51;
p) SEQ ID NOs: 9 and 35;
q) SEQ ID NOs: 9 and 39;
r) SEQ ID NOs: 9 and 43;
s) SEQ ID NOs: 9 and 47;
t) SEQ ID NOs: 15 and 35;
u) SEQ ID NOs: 15 and 39;
v) SEQ ID NOs: 15 and 43;
w) SEQ ID NOs: 15 and 47; or
x) SEQ ID NOs: 15 and 51.

### Pharmaceutical Compositions

Another aspect of the present disclosure is a pharmaceutical composition comprising as an active ingredient (or as the sole active ingredient) an anti-TNFα antibody or antigen-binding portion thereof, bispecific binding molecule, or immunoconjugate of the present disclosure. In some embodiments, the pharmaceutical compositions are intended for amelioration, prevention, and/or treatment of an autoimmune or inflammatory condition, e.g., a condition described herein.

Generally, the antibodies and antigen-binding portions, bispecific binding molecules, and immunoconjugates of the present disclosure are suitable to be administered as a formulation in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present disclosure and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP (good manufacturing practices) conditions.

A pharmaceutical composition of the present disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Formulations of a pharmaceutical composition suitable for parenteral administration (e.g., subcutaneous administration) typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation.

### Therapeutic Uses of Antibodies and Compositions of the Present Disclosure

In some embodiments, an anti-TNFα antibody or antigen-binding portion, bispecific binding molecule, or immunoconjugate of the present disclosure is used to treat a condition in a patient, e.g., a cancer, a pulmonary condition, an intestinal condition, or a cardiac condition. In certain embodiments, the condition is an autoimmune or inflammatory condition. The patient may be a mammal, e.g., a human.

In some embodiments, the patient has a condition selected from arthritis (e.g., rheumatoid arthritis, psoriatic arthritis, gouty arthritis, juvenile idiopathic arthritis (e.g., polyarticular juvenile idiopathic arthritis), spondyloarthritis (e.g., peripheral or axial spondyloarthritis), osteoarthritis, oligoarthritis, erosive polyarthritis, or enthesitis related arthritis), Crohn's disease, ulcerative colitis, enterocolitis, inflammatory bowel disease, psoriasis (e.g., plaque psoriasis, pustular psoriasis, psoriasis vulgaris, or nail psoriasis), ankylosing spondylitis, rheymatoid spondylitis, hidradenitis suppurativa, pyoderma gangrenosum, Netherton syndrome, Dupuytren's disease, Hermansky-Pudlak syndrome, atopic dermatitis, asthma, allergy, uveitis (e.g., panuveitis or non-infectious uveitis), age-related macular degeneration, diabetic retinopathy, scleritis, Rasmussen encephalitis, asthma, sarcoidosis (e.g., cutaneous sarcoidosis), arteritis (e.g., Takayasu's arteritis or giant cell arteritis), vasculitis, Kawasaki disease, Behcet's disease, pouchitis, hepatitis, nephrotic syndrome, atherosclerosis, glomerulosclerosis (e.g., focal segmental glomerulosclerosis), multiple sclerosis, mucopolysaccharidosis (e.g., type I, II, or IV), diabetes mellitus (e.g., Type I diabetes or autoimmune diabetes), myocardial inflammation, interstitial cystitis, inflammatory bone disorder, osteomyelitis (e.g., chronic nonbacterial osteomyelitis or chronic recurrent multifocal osteomyelitis), osteoporosis, sciatica, chronic granulomatous disease, systemic lupus erythematosus (SLE), an autoimmune thyroid disorder (e.g., Hashimoto's disease), transplant rejection, graft-versus-host disease, obstructive sleep apnea, amyloidosis, a neuropsychiatric disorder (e.g., depression), and a neurodegenerative disorder (e.g., Alzheimer's disease). In some embodiments, the condition may be a chronic or acute condition, and/or may be an adult or pediatric condition.

In certain embodiments, the autoimmune or inflammatory condition is rheumatoid arthritis, psoriatic arthritis, plaque psoriasis, ankylosing spondylitis, axial spondyloarthritis, Crohn's disease, ulcerative colitis, hidradenitis suppurativa, polyarticular juvenile idiopathic arthritis, panuveitis, or Alzheimer's disease.

In some embodiments, a patient to be treated with an anti-TNFα antibody or antigen-binding portion, bispecific binding molecule, or immunoconjugate of the present disclosure has received prior treatment for the condition to be treated (e.g., autoimmune or inflammatory condition). In other embodiments, the patient has not received such prior treatment. In some embodiments, the patient has failed on a prior treatment for the condition (e.g., a prior TNFα-targeting treatment)

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

An anti-TNFα antibody or antigen-binding portion, bispecific binding molecule, or immunoconjugate of the present disclosure may be administered in a therapeutically effective amount to a patient with a condition described herein. “Therapeutically effective amount” refers to the amount of the therapeutic agent being administered that will relieve to some extent one or more of the symptoms of the disorder being treated, and/or result in clinical endpoint(s) desired by healthcare professionals.

An anti-TNFα antibody or antigen-binding portion, bispecific binding molecule, or immunoconjugate of the present disclosure may be administered without additional therapeutic treatments, i.e., as a stand-alone therapy (monotherapy). Alternatively, treatment with an anti-TNFα antibody or antigen-binding portion, bispecific binding molecule, or immunoconjugate of the present disclosure may include at least one additional therapeutic treatment (combination therapy). In some embodiments, an anti-TNFα antibody or antigen-binding portion, bispecific binding molecule, or immunoconjugate may be co-administered or formulated with another medication/drug for the treatment of the relevant condition (e.g., autoimmune or inflammatory condition).

In some embodiments, an anti-TNFα antibody or antigen-binding portion, bispecific binding molecule, or immunoconjugate of the present disclosure is administered in combination with one or more agents or treatments selected from methotrexate, prednisone, betamethasone, Enstilar®, calcipotriol, metronidazole, azathioprine, tacrolimus, hydroxychloroquine, an oral glucocorticosteroid, a non-steroidal anti-inflammatory drug (NSAID), baricitinib, ciprofloxacin, leflunomide, exenatide, teriparatide, sulfasalazine, thiopurine, 6 mercaptopurine, 2′-fucosyllactose, abatacept, etanercept, infliximab, rituximab, tocilizumab, vedolizumab, golimumab, certolizumab, ustekinumab, sarilumab, andecaliximab, anakinra, an NK cell lectin-like-receptor subfamily K antagonist (e.g., tesnatilimab), anti-thymocyte globulin, IL-2, a homocysteine modulator (e.g., vitamin B12, vitamin B6, or folic acid), and radiation.

It is understood that the antibodies and antigen-binding portions thereof, bispecific binding molecules, and immunoconjugates of the present disclosure may be used in a method of treatment as described herein, may be for use in a treatment as described herein, and/or may be for use in the manufacture of a medicament for a treatment as described herein. It is also understood that the therapies described herein may be carried out not only using the anti-TNFα antibodies or antigen-binding portions thereof, bispecific binding molecules, or immunoconjugates of the present disclosure, but also using any related pharmaceutical compositions described herein. The present disclosure also provides kits and articles of manufacture comprising the antibodies and antigen-binding portions thereof, bispecific binding molecules, immunoconjugates, or pharmaceutical compositions described herein.

### Dose and Route of Administration

The antibodies or antigen-binding portions thereof, bispecific binding molecules, and immunoconjugates of the present disclosure may be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the antibodies, bispecific binding molecules, and immunoconjugates are being administered as a stand-alone treatment or in combination with one or more additional treatments for autoimmune and/or inflammatory diseases.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the present disclosure are generally dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of the present disclosure may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens are well-known in the relevant art and would be understood to be encompassed by the skilled artisan once provided the teachings disclosed herein.

An effective amount for therapy may be measured by its ability to stabilize disease progression and/or ameliorate symptoms in a patient, and preferably to reverse disease progression. The ability of an antibody, antigen-binding portion, bispecific binding molecule, immunoconjugate, or pharmaceutical composition of the present disclosure to inhibit an autoimmune or inflammatory disease may be evaluated by in vitro assays, e.g., as described in the examples, as well as in suitable animal models that are predictive of the efficacy in humans. Suitable dosage regimens will be selected in order to provide an optimum therapeutic response in each particular situation, for example, administered as a single bolus or as a continuous infusion, and with possible adjustment of the dosage as indicated by the exigencies of each case.

The antibodies or antigen-binding portions thereof, bispecific binding molecules, immunoconjugates, and pharmaceutical compositions of the present disclosure may be administered by any method for administering peptides, proteins or antibodies accepted in the art, and are typically suitable for parenteral administration. As used herein, “parenteral administration” includes any route of administration characterized by physical breaching of a tissue of a subject and administration through the breach in the tissue, thus generally resulting in the direct administration into the blood stream, into muscle, or into an internal organ. Parenteral administration thus includes, but is not limited to, administration by injection, by application through a surgical incision, by application through a tissue-penetrating non-surgical wound, and the like. In particular, parenteral administration is contemplated to include, but is not limited to, intravenous, subcutaneous, intraperitoneal, intramuscular, intrasternal, intraarterial, intrathecal, intraurethral, intracranial, and intrasynovial injection or infusions. In a particular aspect, the antibodies or antigen-binding portions, bispecific binding molecules, immunoconjugates, or pharmaceutical compositions described herein are administered subcutaneously.

### Diagnostic Uses and Compositions

The antibodies and antigen-binding portions of the present disclosure also are useful in diagnostic processes (e.g., in vitro, ex vivo). For example, the antibodies and antigen-binding portions can be used to detect and/or measure the level of TNFα in a sample from a patient (e.g., a tissue sample, or a body fluid sample such as an inflammatory exudate, blood, serum, bowel fluid, saliva, or urine). Such detection may, for example, aid with prediction of whether or not the patient will be responsive to TNFα antibody therapy. Suitable detection and measurement methods include immunological methods such as flow cytometry, enzyme-linked immunosorbent assays (ELISA), chemiluminescence assays, radioimmunoassays, and immunohistology. The present disclosure further encompasses kits (e.g., diagnostic kits) comprising the antibodies and antigen-binding portions described herein.

### Articles of Manufacture and Kits

The present disclosure also provides articles of manufacture, e.g., kits, comprising a one or more containers (e.g., single-use or multi-use containers) containing a pharmaceutical composition of the anti-TNFα antibody or antigen-binding portion, bispecific binding molecule, or immunoconjugate of the present disclosure; optionally an additional biologically active molecule (e.g., another therapeutic agent); and instructions for use. The anti-TNFα antibody or antigen-binding portion, bispecific binding molecule, or immunoconjugate, and optional additional biologically active molecule, can be packaged separately in suitable packing such as a vial or ampoule made from non-reactive glass or plastic. In certain embodiments, the vial or ampoule holds lyophilized powder comprising the anti-TNFα antibody or antigen-binding portion, bispecific binding molecule, or immunoconjugate and/or the additional biologically active molecule. In certain embodiments, the vial or ampoule holds a concentrated stock (e.g., 2×, 5×, 10× or more) of the anti-TNFα antibody or antigen-binding portion, bispecific binding molecule, or immunoconjugate and/or the biologically active molecule. In certain embodiments, the articles of manufacture such as kits include a medical device for administering the anti-TNFα antibody or antigen-binding portion, bispecific binding molecule, or immunoconjugate and/or the biologically active molecule (e.g., a syringe and a needle); and/or an appropriate diluent (e.g., sterile water and normal saline). The articles of manufacture may further include instructions for using the anti-TNFα antibody or antigen-binding portion, bispecific binding molecule, or immunoconjugate, and optionally the additional biologically active molecule, in a method described herein. The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

In order that the present disclosure may be better understood, the following examples are set forth. These examples are for purposes of illustration only and are not to be construed as limiting the scope of the present disclosure in any manner.

### EXAMPLES

### Example 1. Design, Expression, and Purification of Monovalent Ab1

To eliminate the formation of large immune complexes (IC) via the cross-linking of TNFα by Ab1, we created a monovalent antibody format consisting of a single Ab1 antigen-binding fragment (Fab) and a modified heterodimeric constant domain fragment (Fc). This approach employed knobs-into-holes (KIH) technology that enables the efficient assembly and purification of heterodimeric antibodies, while minimizing the formation of the undesired homodimer species. Monovalent Ab1, termed AF-M2630, consisted of three distinct polypeptide chains: (1) Ab1 kappa light chain, (2) Ab1 heavy chain with T366S, L368A and Y407V mutations (“holes”) introduced into the CH3 domain, and (3) a truncated heavy chain lacking the variable region and CH1 domain with a T366W mutation (“knob”) in the CH3 domain. Additionally, to further stabilize the monovalent construct, Y349C was introduced into Ab1 “hole” chain and S354C was introduced into the “knob” chain. Thus, AF-M2630 contains a single variable region to prevent the cross-linking of TNFα, as well as a fully functional Fc domain, to preserve both the favorable pharmacokinetic and effector function properties of Ab1.

### Materials and Methods

### Antibody Expression and Purification

The heavy and light chain variable regions of Ab1 were synthesized as gBlocks (IDT) and cloned into pFUSE-CHIg-hG1 and pFUSE2-CLIg-hk vectors (Invivogen), respectively, by Gibson assembly (NEB). DNA was harvested from 5-alpha competent F. coli (High Efficiency) (NEB) cultures using dsDNA isolation kits (Qiagen) and the sequences were confirmed.

Antibodies were expressed in FreeStyle™ 293-F cells (ThermoFisher Scientific) maintained in 293 Freestyle medium (ThermoFisher Scientific). The cells were transfected with purified plasmids using 293fectin (ThermoFisher Scientific) according to manufacturer's protocol, including splitting conditions for the cells. For bivalent constructs, the light chain was transfected at 4:1 ratio relative to the heavy chain. For monovalent constructs, the light chain, truncated heavy chain (knob), and heavy chain (hole) were transfected at a 4:2:1 ratio, respectively. Culture supernatants were harvested four days after transfection and were filtered through a 0.22 mm Steriflip (EMD Millipore).

Antibodies were purified using a 1 mL HiTrap MabSelect SuRe column (GE Healthcare) and all steps were performed using a flow rate of 0.5 mL/min. The column was equilibrated with 10 mL of 20 mM sodium phosphate, 150 mM NaCl, pH 7.4, the sample was loaded and subsequently, the column was washed with 10 mL of 20 mM sodium phosphate, 500 mM NaCl, pH 7.4. Next, the column was washed with 10 mL of 20 mM sodium phosphate, 150 mM NaCl, pH 7.4 and antibody was eluted with 5 mL sodium acetate, pH 3.5. In order to rapidly neutralize the antibody, it was eluted into a tube containing 220 μL 1M Tris, pH 9. Finally, the antibody was buffer exchanged into PBS using an Amicon Ultra-15 centrifugal filter device (30,000 molecular weight cut-off) per manufacturer's directions.

### SDS-PAGE

Samples were diluted in 4X Bolt LDS sample buffer (ThermoFisher Scientific) with and without 10X Bolt sample reducing agent (ThermoFisher Scientific), the samples were heated for 20 min at 80° C., and 2 μg per well was loaded on a 15 well Bolt 4-12% Bis-Tris Plus, 1.0 mm, mini protein gel (Invitrogen). The samples were resolved at 150V for 1 h using 1X Bolt MOPS SDS running buffer (ThermoFisher Scientific) and at completion of electrophoresis, the gel was rinsed three times, 5 min each, with deionized water. The gels were stained in 20 mL of SimplyBlue™ SafeStain (ThermoFisher Scientific) for 1 h at 25° C., de-stained in deionized water for 1 h at 25° C. and then placed in fresh deionized water and de-stained 18 h at 25° C. The gels were imaged using the iBright FL1000 Imaging System (ThermoFisher Scientific).

### Results

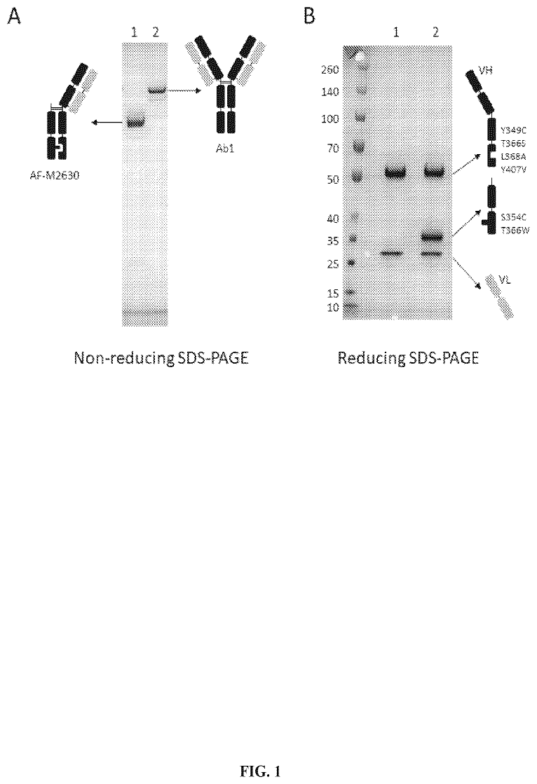

HEK293 cells were transiently transfected with DNA encoding the heavy and light chains of Ab1 or with DNA encoding the three distinct chains of AF-M2630, and immunoglobulin was isolated from the culture supernatants using protein A. Following isolation, the immunoglobulins were characterized by SDS-PAGE under reducing and non-reducing conditions. AF-M2630 contained a single predominant band under non-reducing conditions (FIG. 1A, lane 1). As expected, Ab1 was also predominantly a single band (FIG. 1A, lane 2), though a minor faster migrating species was also observed. When the samples were characterized under reducing conditions, two distinct bands were observed for Ab1 (FIG. 1B, lane 1: heavy and light chain) while three distinct bands were observed for AF-M2630 (FIG. 1B, lane 2; heavy chain “holes,” truncated heavy chain “knobs,” and light chain). Collectively, these data demonstrate the expression and one-step purification of Ab1 and AF-M2630, and furthermore, demonstrate the efficiency of the KIH approach for generating AF-M2630.

### Example 2. AF-M2630 Binds TNFα with High Affinity

The binding affinity of Ab1 and AF-M2630 were characterized by ELISA using plates coated with TNFα.

### Materials and Methods

### Antibody Binding to Human TNFα

A Costar 3366 96-well plate was coated with 1 μg/mL recombinant human TNFα (Genscript) in PBS for 1 h. Subsequently, the plate was washed once (all wash steps were performed with PBS, 0.05% Tween 20) and was then blocked with 100 μL of PBS, 1% (w/v) BSA (Fisher Scientific) for 30 min at 25° C. Antibodies were diluted serially 3-fold in PBS, 1% (w/v) BSA and were incubated for 1 h at 25° C. The plates were washed four times and 50 μL/well of goat anti-human-Fc-HRP (SouthernBiotech) was added for 1 h at 25° C. The plates were washed four times and developed using 50 μL/well of Ultra-TMB-ELISA substrate (ThermoFisher Scientific). The reaction was terminated by the addition of 50 μL/well of 2N H₂SO₄ and the absorbance at 450 nm was quantitated using a Spectramax Plus 384 microplate reader (Molecular Devices).

### Results

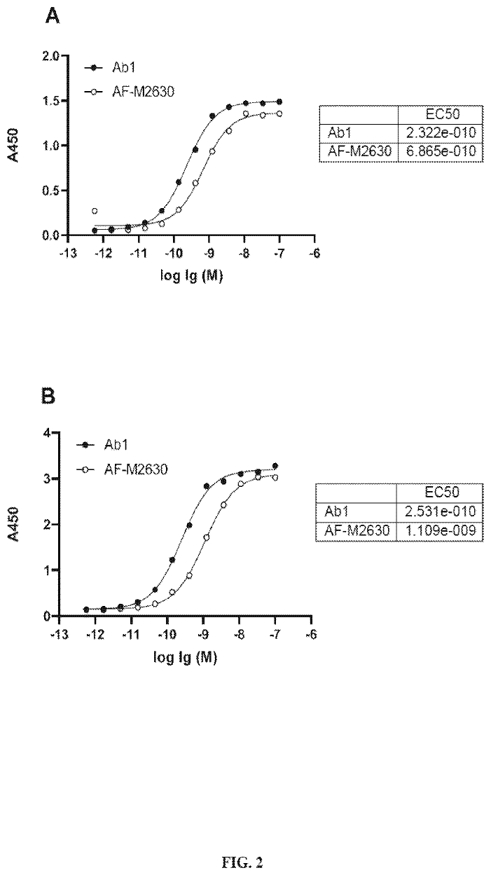

Ab1 and AF-M2630 both bound TNFα with high affinity in a saturable, concentration-dependent manner (FIG. 2A). The binding of Ab1 (EC50=232 pM) was approximately 3-fold stronger than the binding of AF-M2630 (EC50=687 pM). The enhanced binding of Ab1 relative to the monovalent AF-M2630 construct is consistent with Ab1 displaying an avidity effect (multivalent interaction) in this assay format. To further characterize the potential contribution of avidity the antigen-down ELISA was modified. Specifically, after allowing Ab1 or AF-M2630 to bind to immobilized TNFα, unbound antibody was removed with a brief rinse and the plates were subjected to a prolonged 1 h wash prior to adding the secondary detection antibody. Using a prolonged wash step had little impact on Ab1 binding (FIG. 2B, EC50=253 PM) but further diminished the binding of AF-M2630 (FIG. 2B, EC50=1.1 nM). These data further support the notion that Ab1 binding is enhanced by avidity in this assay format.

### Example 3. AF-M2630 does not Form Large Precipitating IC

After confirming that AF-M2630 retained high affinity binding to TNFα, we characterized both Ab1 and AF-M2630 for their ability to form large, precipitating IC using a modified Ouchterlony double diffusion assay (Kohno et al., Journal of Investigative Dermatology Symposium Proceedings (2007) 12:5-8). For this study, we defined large IC as IC comprising two or more TNFα molecules cross-linked by three or more antibodies, and small IC as IC consisting of a single homotrimer TNFα molecule bound by 1-3 antibody molecules. Large IC may be soluble or precipitating, depending on the size of the complex.

### Materials and Methods

The central well of a rosette pattern of wells in an agarose polyethylene glycol micro-Ouchterlony plate (MP Biomedical, LLC) was loaded with 2.5 μg of recombinant human TNFα (Genscript) or with 2.5 μg of goat anti-human Fc polyclonal antibody (SouthernBiotech) and 5 μg of Ab1, Ab1 variant, or control IgG1 was added to the surrounding wells. The gel was placed at 37° C. for 16 h at which time it was removed from the case and incubated in multiple changes of water at 25° C. for 3 h. To visualize protein, the gel was stained with SimplyBlue SafeStain (ThermoFisher Scientific) at 25° C., per manufacturer's directions.

### Results

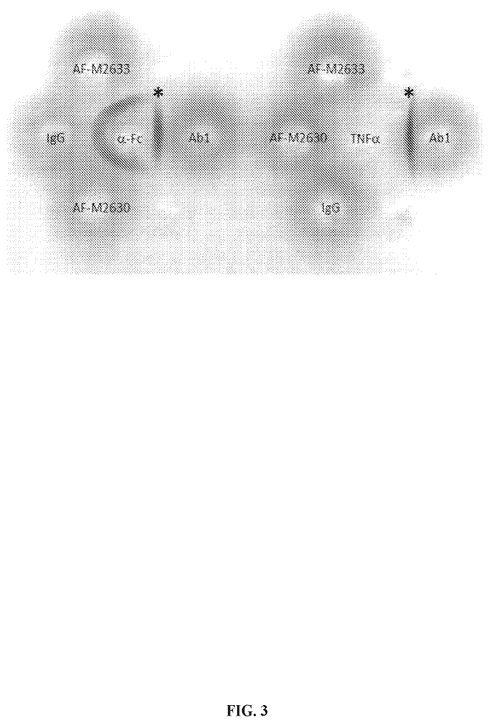

To increase the visibility of the precipitating large IC (precipitin line), we treated the agarose gel with a protein stain. Precipitation of protein complexes generates darker protein staining. Ab1 formed precipitin lines with both TNFα (FIG. 3, right, denoted with asterisk) and with polyclonal anti-human Fc (FIG. 3, left) while AF-M2630 only formed precipitin lines with anti-human Fc (FIG. 3, compare left vs right). A control antibody unreactive with TNFα and a second monovalent Ab1 construct, termed AF-M2633, formed precipitin lines with anti-human Fc (FIG. 3, left), but not with TNFα (FIG. 3, right). These results demonstrate that Ab1 cross-links TNFα and forms large precipitating IC under certain conditions. By contrast, AF-M2630, with a single TNFα binding site, cannot form large IC.

### Example 4. AF-M2630 Forms Small IC with TNFα, Regardless of TNFα Stoichiometry

Next, the ability of Ab1 and AF-M2630 to form small IC or non-precipitating large IC was evaluated. For this study, we defined large IC as IC comprising two or more TNFα molecules cross-linked by three or more antibodies, and small IC as IC consisting of a single homotrimer TNFα molecule bound by 1-3 antibody molecules. Large IC may be soluble or precipitating, depending on the size of the complex.

### Materials and Methods

### Analytical Size Exclusion Chromatography

Samples containing antibody, TNFα, or mixtures of antibodies and TNFα at 10:1 and 1:1 molar ratios (Ig:TNFα) were incubated overnight at 4° C. Prior to analysis, each sample was briefly centrifuged at 14000 rpm for 30 s to remove large precipitate. Subsequently, 20 μL of sample containing 20-50 μg of antibody was applied to a TSKgel UP-SW3000, 2 μm, 30 cm×4.6 mm column on an Agilent Technologies 1200 series instrument. Peaks were resolved using PBS, pH 7.4 as the mobile phase solution at a flow rate of 0.35 mL/min and protein was detected at 290 nm. Peak area was calculated using the Agilent software.

### Results

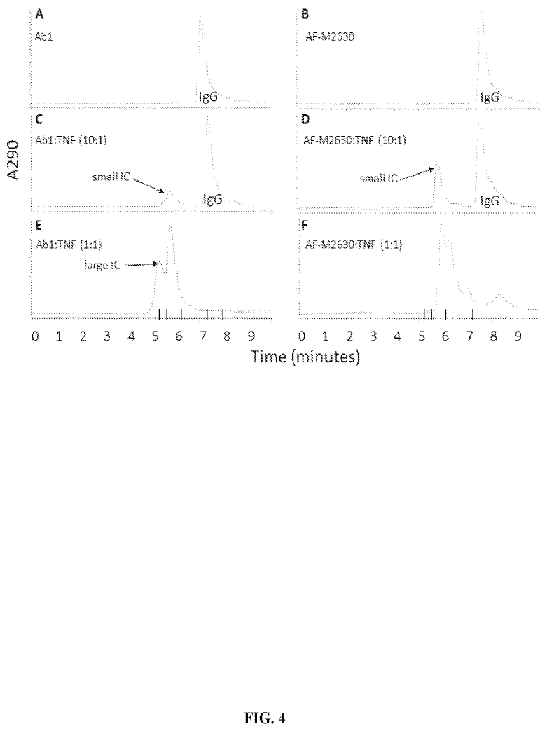

Ab1 and AF-M2630 were incubated with TNFα at varying stoichiometries and the mixtures were analyzed by analytical size exclusion chromatography (SEC) to characterize the ICs formed. Initial analysis of the individual components demonstrated that each eluted predominantly as a single peak. Ab1 (MW˜150 kDa) eluted at 7.3 min (FIG. 4A), AF-M2630 (MW 100 kDa) at 7.6 min (FIG. 4B) and TNFα (MW 51 kDa) at 8.3 min (not shown).

Subsequently, each antibody was mixed with TNFα with antibody in great excess (10:1 molar ratio) and incubated at 4° C. for 12-15 h to drive the formation of IC. Under these conditions two peaks were observed for both the Ab1 and the AF-M2630 samples. For Ab1, the peaks displayed retention times of 5.7 min and 7.3 min, respectively (FIG. 4C), with the peak eluting at 7.3 min being consistent with free, unbound antibody. The retention time of the early eluting first peak was between the thyroglobulin (MW 669 kDa) and ferritin (440 kDa) protein standards and is consistent with a small IC consisting of Ab1 and TNFα in a 3:1 complex (˜500 kDa).

Similar results were observed when AF-M2630 was incubated at a 10:1 molar ratio with TNFα. A peak associated with free, unbound AF-M2630 was observed at 7.6 min and a second, earlier eluting IC peak was observed at 5.9 min (FIG. 4D). The later elution time of AF-M2630 IC relative to Ab1 IC of similar stoichiometry (5.9 min vs 5.7 min) is expected as AF-M2630 is 50 kDa smaller than Ab1.

When Ab1 was preincubated with TNFα at a lower stoichiometry (1:1 molar ratio) a new IC peak eluting as early as 5.3 min, similar to thyroglobulin dimer (1,338 kDa), was observed (FIG. 4E). This peak displayed a shorter retention time than the IC formed when Ab1 was incubated with TNFα at a 10:1 molar ratio. Thus, it is likely that the new peak comprises large IC containing ≥2 TNFα molecules and three or more Ab1 molecules. At the lower Ig/TNFα stoichiometry all Ab1 was bound and no peak corresponding to unbound Ab1 was observed at 7.3 min.

When AF-M2630 was preincubated with TNFα at a lower stoichiometry (1:1 molar ratio) four peaks were observed, with retention times of 5.9 min, 6.1 min, 7.0 min, and 8.3 min (FIG. 4F). However, in contrast to the Ab1 lower stoichiometry sample, none of the peaks had a shorter retention time than was observed when excess AF-M2630 was preincubated with TNFα (10:1 molar ratio). Instead, the data are consistent with the formation of heterogeneous small IC consisting of AF-M2630 and TNFα in 3:1 (5.9 min), 2:1 (6.1 min), and 1:1 (7.0 min) ratios. The 8.3 min retention time of the fourth peak is longer than free AF-M2630 (7.6 min) and is consistent with the elution profile of TNFα alone (not shown).

Collectively, these data are consistent with the Ouchterlony double diffusion assay results. Ab1 can form large IC under certain conditions while its monovalent derivative, AF-M2630, prevents the formation of large IC, regardless of the stoichiometry of immunoglobulin and TNFα.

### Example 5. Engineering a Monovalent pH Switch

A novel class of antibodies has been described that bind antigens in the extracellular environment while releasing the target in acidic (pH 6.0) endosomal compartments following internalization into the cell (Igawa et al., Nat Biotechnol. (2010) 28:1203-7). The use of such antibodies is predicted to result in the dissociation and delivery of antigen to lysosomes, whereas the antibody will be recycled to the cell surface by FcRn. This novel class of antibodies, also referred to as pH switch antibodies, typically integrate histidine residues into complementarity determining regions (CDRs) to create pH dependent binding.

To inhibit the lysosomal trafficking that occurs with many internalized antibodies, we incorporated a pH-sensitive antigen binding function (pH switch) into AF-M2630. The pH switch allows the antibody to bind and neutralize both serum (soluble) and membrane-associated TNFα, while also enabling dissociation following internalization into the acidic endosomal environment (˜pH 6.0). With other pH switch antibodies, dissociated antibody is recycled to the cell surface via the FcRn, while the antigen is trafficked to the lysosomes for degradation.

The goal of the studies described in this example was to identify pH switch variant(s) that works best in a monovalent format, because the monovalent format prevents the formation of large IC. The optimal candidate will preserve high affinity binding of TNFα at pH 7.4 (K_{D}<50 nM or 100 nM), while displaying a large increase in dissociation rate at pH 6.0 (>10-fold faster k_{dis} than Ab1, and/or k_{dis}>2e-004 s⁻¹). Retention of high affinity at pH 7.4 may be important to enable the monovalent format to bind and neutralize both TNFα and mTNFα in the absence of avidity. The high affinity interaction at pH 7.4 leads to the internalization and targeted lysosomal degradation of TNFα (both IC and mTNFα) while the large increase in dissociation rate at pH 6.0 enables the antibody to release from the TNFα in the endocytic pathway and recycle back to the cell surface. It is important to evaluate the strength (enhancement of k_{dis}) of the pH switches in a monovalent format, as the multivalent interaction of bivalent IgG (avidity) can counteract pH sensitivity (Gera et al., PLoS One (2012) 7: e48928; Schröter et al., MABs (2015) 7 (1): 138-51; Traxlmayr et al., Biotechnol J (2014) 9:1013-22). Specifically, certain pH switches will have significantly faster dissociation rates in the monovalent format as compared to the typical bivalent IgG format.

### Materials and Methods

The ELISA was performed as described in Example 2, with minor modifications. For assays using a prolonged pH 7.4 or pH 6.0 dissociation step, after the plate was washed four times following the incubation of test antibodies, it was submerged in 500 mL of either PBS, 0.05% Tween 20, pH 7.4 or PBS, 0.05% Tween 20, pH 6.0 for 1 h at 25° C. To ensure thorough washing, buffer was aspirated from the plate every 15 min, followed by re-submersion in 500 mL of wash buffer.

### Antibody Binding to Biotinylated Human TNFα (pH 7.4 and pH 6.0 Elution)

A 96-well plate (Costar 3366) was coated with 50 μL of 2 μg/mL goat anti-human Fc (SouthernBiotech) in PBS for 1 h at 25° C. Subsequently, the plate was washed once (all wash steps were performed with PBS, 0.05% Tween 20) and was then blocked with 100 μL of PBS, 1% (w/v) BSA (Fisher Scientific) for 1 h at 25° C. Next, 50 μL of 1 μg/mL test article antibody diluted in PBS, 1% (w/v) BSA was added and incubated for 1 h at 25° C. The plate was washed four times and 50 μL of biotinylated human TNFα (Acro Biosystems) diluted serially, 3-fold in PBS, 1% (w/v) BSA was incubated for 1 h at 25° C. The plate was washed once and was submerged in 500 mL of either PBS, 0.05% Tween 20, pH 7.4 or PBS, 0.05% Tween 20, pH 6.0 for 1 h at 25° C. prior to the addition of 50 μL of neutravidin-HRP (ThermoFisher Scientific) diluted 1:2000 in PBS, 1% (w/v) BSA for 1 h at 25° C. To ensure thorough washing, buffer was aspirated from the plate every 15 min, followed by re-submersion in 500 mL of wash buffer. The plate was washed four times and was developed using 50 μL/well of Ultra-TMB-ELISA substrate (ThermoFisher Scientific). The reaction was terminated by the addition of 50 μL/well of 2N H₂SO₄ and the absorbance at 450 nm was quantitated using a Spectramax Plus 384 microplate reader (Molecular Devices).

### Results

We evaluated previously described Ab1 variants (Schröter et al., supra) that were engineered to retain high affinity TNFα binding at pH 7.4 while displaying enhanced dissociation at acidic pH (pH 4.5-6.0). First, Ab1, monovalent Ab1 (AF-M2630), and the monovalent format of three heavy chain variable region (VH), six light chain variable region (VL) and fifteen combinatorial VH/VL pH switch variants were transiently expressed and purified using protein A chromatography.

The compositions of the variants described in these studies are summarized in Table 1.

TABLE 1
Sequences of Ab1, AF-M2630 (monovalent Ab1), and variants Chain 2

| | | | Heavy Chain 1 | | VH | | Heavy Chain 2 | | | Light Chain | | VL | |
|---|---|---|---|---|---|---|---|---|---|---|---|---|---|
| | | Heavy | DNA | Protein | DNA | Protein | DNA | Protein | Light | DNA | Protein | DNA | Protein |
| Construct | Valency | Chain | SEQ | SEQ | SEQ | SEQ | SEQ | SEQ | Chain | SEQ | SEQ | SEQ | SEQ |
| Ab1 | 2 | WT | 1 | 2 | 3 | 4 | — | — | WT | 5 | 6 | 7 | 8 |
| AF-M2630 | 1 | WT | 9 | 10 | 3 | 4 | 11 | 12 | WT | 5 | 6 | 7 | 8 |
| AF-B2631 | 2 | VH1 | 13 | 14 | 15 | 16 | — | — | WT | 5 | 6 | 7 | 8 |
| AF-M2631 | 1 | VH1 | 17 | 18 | 15 | 16 | 11 | 12 | WT | 5 | 6 | 7 | 8 |
| AF-B2632 | 2 | VH2 | 19 | 20 | 21 | 22 | — | — | WT | 5 | 6 | 7 | 8 |
| AF-M2632 | 1 | VH2 | 23 | 24 | 21 | 22 | 11 | 12 | WT | 5 | 6 | 7 | 8 |
| AF-B2633 | 2 | VH3 | 25 | 26 | 27 | 28 | — | — | WT | 5 | 6 | 7 | 8 |
| AF-M2633 | 1 | VH3 | 29 | 30 | 27 | 28 | 11 | 12 | WT | 5 | 6 | 7 | 8 |
| AF-B2634 | 2 | WT | 1 | 2 | 3 | 4 | — | — | VL1 | 31 | 32 | 33 | 34 |
| AF-M2634 | 1 | WT | 9 | 10 | 3 | 4 | 11 | 12 | VL1 | 31 | 32 | 33 | 34 |
| AF-B2635 | 2 | WT | 1 | 2 | 3 | 4 | — | — | VL2 | 35 | 36 | 37 | 38 |
| AF-M2635 | 1 | WT | 9 | 10 | 3 | 4 | 11 | 12 | VL2 | 35 | 36 | 37 | 38 |
| AF-B2636 | 2 | WT | 1 | 2 | 3 | 4 | — | — | VL3 | 39 | 40 | 41 | 42 |
| AF-M2636 | 1 | WT | 9 | 10 | 3 | 4 | 11 | 12 | VL3 | 39 | 40 | 41 | 42 |
| AF-B2637 | 2 | WT | 1 | 2 | 3 | 4 | — | — | VL4 | 43 | 44 | 45 | 46 |
| AF-M2637 | 1 | WT | 9 | 10 | 3 | 4 | 11 | 12 | VL4 | 43 | 44 | 45 | 46 |
| AF-B2638 | 2 | WT | 1 | 2 | 3 | 4 | — | — | VL5 | 47 | 48 | 49 | 50 |
| AF-M2638 | 1 | WT | 9 | 10 | 3 | 4 | 11 | 12 | VL5 | 47 | 48 | 49 | 50 |
| AF-B2639 | 2 | WT | 1 | 2 | 3 | 4 | — | — | VL6 | 51 | 52 | 53 | 54 |
| AF-M2639 | 1 | WT | 9 | 10 | 3 | 4 | 11 | 12 | VL6 | 51 | 52 | 53 | 54 |
| AF-B2640 | 2 | VH1 | 13 | 14 | 15 | 16 | — | — | VL1 | 31 | 32 | 33 | 34 |
| AF-M2640 | 1 | VH1 | 17 | 18 | 15 | 16 | 11 | 12 | VL1 | 31 | 32 | 33 | 34 |
| AF-B2641 | 2 | VH1 | 13 | 14 | 15 | 16 | — | — | VL2 | 35 | 36 | 37 | 38 |
| AF-M2641 | 1 | VH1 | 17 | 18 | 15 | 16 | 11 | 12 | VL2 | 35 | 36 | 37 | 38 |
| AF-B2642 | 2 | VH1 | 13 | 14 | 15 | 16 | — | — | VL3 | 39 | 40 | 41 | 42 |
| AF-M2642 | 1 | VH1 | 17 | 18 | 15 | 16 | 11 | 12 | VL3 | 39 | 40 | 41 | 42 |
| AF-B2643 | 2 | VH1 | 13 | 14 | 15 | 16 | — | — | VL4 | 43 | 44 | 45 | 46 |
| AF-M2643 | 1 | VH1 | 17 | 18 | 15 | 16 | 11 | 12 | VL4 | 43 | 44 | 45 | 46 |
| AF-B2644 | 2 | VH1 | 13 | 14 | 15 | 16 | — | — | VL5 | 47 | 48 | 49 | 50 |
| AF-M2644 | 1 | VH1 | 17 | 18 | 15 | 16 | 11 | 12 | VL5 | 47 | 48 | 49 | 50 |
| AF-B2645 | 2 | VH1 | 13 | 14 | 15 | 16 | — | — | VL6 | 51 | 52 | 53 | 54 |
| AF-M2645 | 1 | VH1 | 17 | 18 | 15 | 16 | 11 | 12 | VL6 | 51 | 52 | 53 | 54 |
| AF-B2646 | 2 | VH2 | 19 | 20 | 21 | 22 | — | — | VL2 | 35 | 36 | 37 | 38 |
| AF-M2646 | 1 | VH2 | 23 | 24 | 21 | 22 | 11 | 12 | VL2 | 35 | 36 | 37 | 38 |
| AF-B2647 | 2 | VH2 | 19 | 20 | 21 | 22 | — | — | VL3 | 39 | 40 | 41 | 42 |
| AF-M2647 | 1 | VH2 | 23 | 24 | 21 | 22 | 11 | 12 | VL3 | 39 | 40 | 41 | 42 |
| AF-B2648 | 2 | VH2 | 19 | 20 | 21 | 22 | — | — | VL4 | 43 | 44 | 45 | 46 |
| AF-M2648 | 1 | VH2 | 23 | 24 | 21 | 22 | 11 | 12 | VL4 | 43 | 44 | 45 | 46 |
| AF-B2649 | 2 | VH2 | 19 | 20 | 21 | 22 | — | — | VL5 | 47 | 48 | 49 | 50 |
| AF-M2649 | 1 | VH2 | 23 | 24 | 21 | 22 | 11 | 12 | VL5 | 47 | 48 | 49 | 50 |
| AF-B2650 | 2 | VH3 | 25 | 26 | 27 | 28 | — | — | VL2 | 35 | 36 | 37 | 38 |
| AF-M2650 | 1 | VH3 | 29 | 30 | 27 | 28 | 11 | 12 | VL2 | 35 | 36 | 37 | 38 |
| AF-B2651 | 2 | VH3 | 25 | 26 | 27 | 28 | — | — | VL3 | 39 | 40 | 41 | 42 |
| AF-M2651 | 1 | VH3 | 29 | 30 | 27 | 28 | 11 | 12 | VL3 | 39 | 40 | 41 | 42 |
| AF-B2652 | 2 | VH3 | 25 | 26 | 27 | 28 | — | — | VL4 | 43 | 44 | 45 | 46 |
| AF-M2652 | 1 | VH3 | 29 | 30 | 27 | 28 | 11 | 12 | VL4 | 43 | 44 | 45 | 46 |
| AF-B2653 | 2 | VH3 | 25 | 26 | 27 | 28 | — | — | VL5 | 47 | 48 | 49 | 50 |
| AF-M2653 | 1 | VH3 | 29 | 30 | 27 | 28 | 11 | 12 | VL5 | 47 | 48 | 49 | 50 |
| AF-B2654 | 2 | VH3 | 25 | 26 | 27 | 28 | — | — | VL6 | 51 | 52 | 53 | 54 |
| AF-M2654 | 1 | VH3 | 29 | 30 | 27 | 28 | 11 | 12 | VL6 | 51 | 52 | 53 | 54 |

SEQ: SEQ ID NO

The CDR sequences for Ab1 and the variants are shown in Table 2.

TABLE 2
CDR sequences of Ab1, AF-M2630 (monovalent Ab1), and variants
Heavy Chain

| | SEQ | CDR1 | SEQ | CDR2 | SEQ | CDR3 |
|---|---|---|---|---|---|---|
| Ab1 | 55 | DYAMH | 56 | AITWNSGHIDYADSVEG | 57 | VSYLSTASSLDY |
| VH1 | 55 | DYAMH | 56 | AITWNSGHIDYADSVEG | 61 | VSYHSTASSLDY |
| VH2 | 55 | DYAMH | 56 | AITWNSGHIDYADSVEG | 62 | VSYLSTAHHLDY |
| VH3 | 55 | DYAMH | 56 | AITWNSGHIDYADSVEG | 63 | VHYHSTASSLDY |

Light Chain

| | SEQ | CDR1 | SEQ | CDR2 | SEQ | CDR3 |
|---|---|---|---|---|---|---|
| Ab1 | 58 | RASQGIRNYLA | 59 | AASTLQS | 60 | QRYNRAPYT |
| VL1 | 64 | RASQGIRNHLA | 59 | AASTLQS | 60 | QRYNRAPYT |
| VL2 | 58 | RASQGIRNYLA | 59 | AASTLQS | 66 | QHYHRAPYT |
| VL3 | 58 | RASQGIRNYLA | 59 | AASTLQS | 67 | QHYHRAPYH |
| VL4 | 58 | RASQGIRNYLA | 59 | AASTLQS | 68 | HHYHRAPYT |
| VL5 | 65 | RASQGIRNHHA | 59 | AASTLQS | 60 | QRYNRAPYT |
| VL6 | 58 | RASQGIRNYLA | 59 | AASTLQS | 69 | QRHNRAPYT |

SEQ: SEQ ID NO

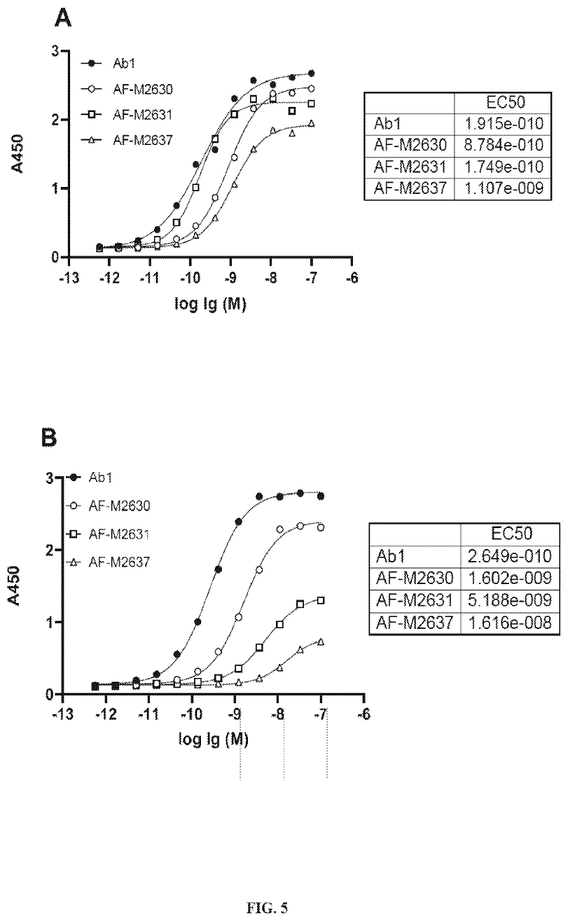

Next, binding was assessed by ELISA using plates coated with TNFα and the initial antibody binding step was performed at pH 7.4 with a subsequent prolonged wash step (dissociation) performed at either pH 7.4 or at pH 6.0. The prolonged pH 6.0 wash step had little impact on the dissociation of Ab1 and the monovalent form of Ab1, AF-M2630 (FIG. 5). The binding EC₅₀ were <2-fold higher and maximal binding at saturation were similar following a pH 6.0 wash as compared to a pH 7.4 wash step.

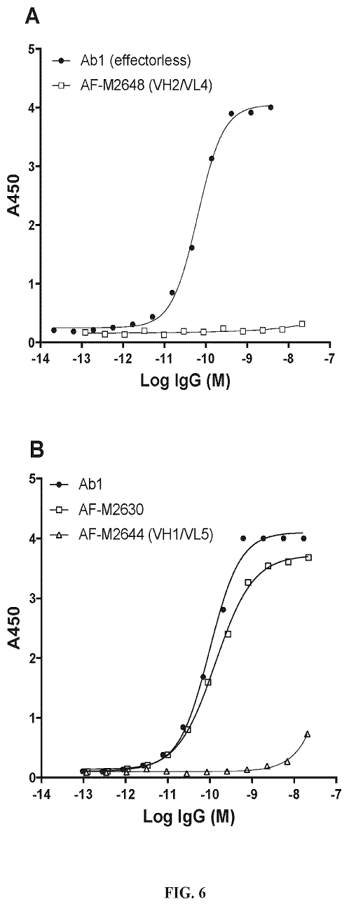

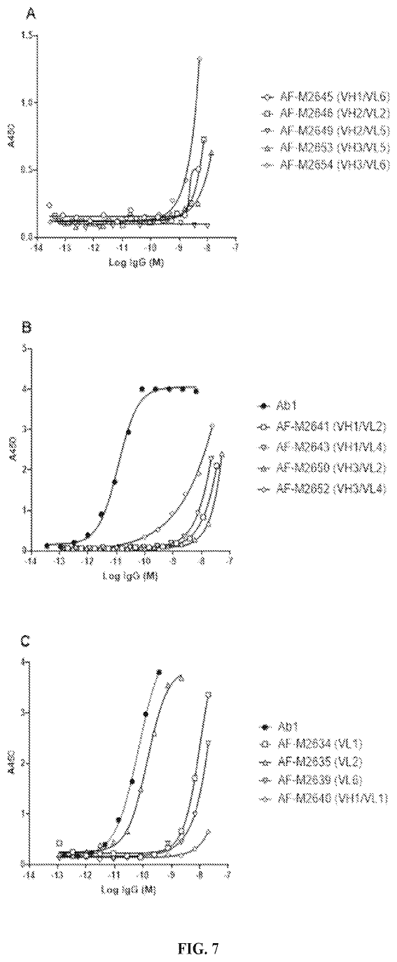

The binding of many of the monovalent pH switch variants at pH 7.4 was diminished significantly relative to the binding of Ab1 (Table 3). Most combinatorial variants, including AF-M2648 (VH2/VL4) and AF-M2644 (VH1/VL5), displayed poor binding. In particular, the binding of twelve of fourteen combinatorial variants, including AF-M2648 (VH2/VL4, FIG. 6A), AF-M2644 (VH1/VL5, FIG. 6B), AF-M2645 (VH1/VL6), AF-M2646 (VH2/VL2), AF-M2649 (VH2/VL5), AF-M2653 (VH3/VL5), AF-M2654 (VH3/VL6) (FIG. 7A), AF-M2641 (VH1/VL2), AF-M2643 (VH1/VL4), AF-M2650 (VH3/VL2), AF-M2652 (VH3/VL4) (FIG. 7B), and AF-M2640 (VH1/VL1) (FIG. 7C) was significantly diminished at pH 7.4. The EC50 of variants that did not achieve saturable binding are designated as “Low” in Table 3. These unexpected data suggest that the original screen used to identify these variants (Schröter et al., supra) had a significant avidity component and highlight the importance of screening in the monovalent format.

TABLE 3
Primary screening summary of Ab1 and variants

| | | Heavy | Light | EC50 (M) | |
|---|---|---|---|---|---|
| Construct | Valency | Chain | Chain | pH 7.4 | pH 6.0 |
| Ab1 | 2 | WT | WT | 7.125E−11 | 6.697E−11 |
| | | | | 2.187E−10 | 2.100E−10 |
| | | | | 1.158E−11 | 1.391E−11 |
| | | | | 9.759E−11 | Not tested |
| | | | | 6.312E−11 | Not tested |
| AF-M2630 | 1 | WT | WT | 2.198E−10 | 3.916E−10 |
| | | | | 1.242E−10 | 1.628E−10 |
| | | | | 1.345E−10 | Not tested |
| AF-M2631 | 1 | VH1 | WT | 4.473E−10 | Low |
| AF-M2632 | 1 | VH2 | WT | 7.983E−10 | Low |
| | | | | 1.299E−09 | Low |
| AF-M2633 | 1 | VH3 | WT | 5.428E−10 | Low |
| | | | | 9.693E−10 | Low |
| AF-M2634 | 1 | WT | VL1 | Low | Low |
| AF-M2635 | 1 | WT | VL2 | 1.426E−10 | 6.232E−10 |
| AF-M2636 | 1 | WT | VL3 | 2.649E−10 | 2.685E−10 |
| AF-M2637 | 1 | WT | VL4 | 5.221E−10 | 1.351E−09 |
| | | | | 3.215E−10 | Low |
| | | | | 5.469E−10 | Low |
| AF-M2638 | 1 | WT | VL5 | Low | Low |
| AF-M2639 | 1 | WT | VL6 | Low | Low |
| AF-M2640 | 1 | VH1 | VL1 | Low | Low |
| AF-M2641 | 1 | VH1 | VL2 | Low | Low |
| AF-M2642 | 1 | VH1 | VL3 | 2.916E−10 | 5.313E−10 |
| | | | | 1.460E−10 | 2.048E−10 |
| AF-M2643 | 1 | VH1 | VL4 | Low | Low |
| AF-M2644 | 1 | VH1 | VL5 | Low | Not tested |
| AF-M2645 | 1 | VH1 | VL6 | Low | Low |
| AF-M2646 | 1 | VH2 | VL2 | Low | Low |
| AF-M2647 | 1 | VH2 | VL3 | Not tested | Not tested |
| AF-M2648 | 1 | VH2 | VL4 | Low | Low |
| AF-M2649 | 1 | VH2 | VL5 | Low | Low |
| AF-M2650 | 1 | VH3 | VL2 | Low | Low |
| AF-M2651 | 1 | VH3 | VL3 | 5.675E−10 | Low |
| | | | | Low | Low |
| AF-M2652 | 1 | VH3 | VL4 | Low | Low |
| AF-M2653 | 1 | VH3 | VL5 | Low | Low |
| AF-M2654 | 1 | VH3 | VL6 | Low | Low |

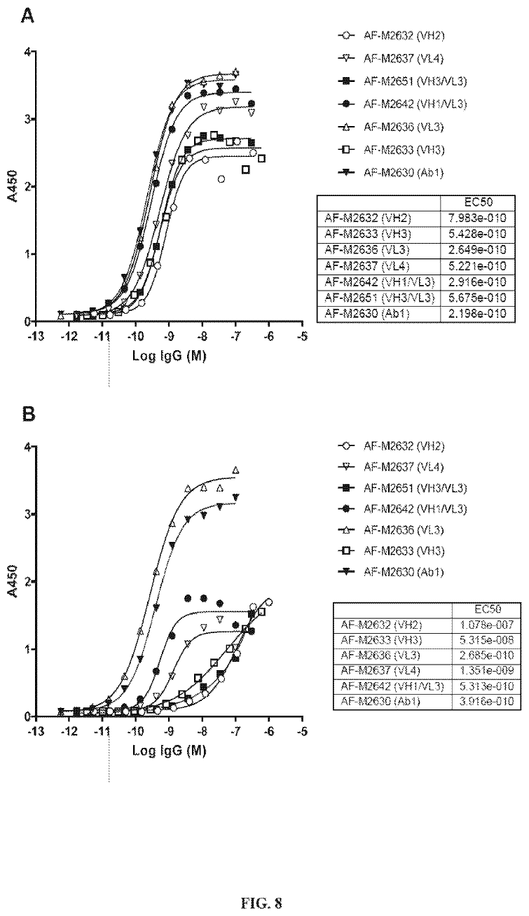

By contrast, six of nine non-combinatorial variants displayed high affinity binding following pH 7.4 washes. Four of these (AF-M2632, AF-M2633, AF-M2636, and AF-M2637) are highlighted in FIG. 8A. Moreover, the binding EC50s of these variants, with the exception of AF-M2636 and AF-M2642, were significantly diminished following a pH 6.0 wash step (FIG. 8B). Although the EC50 of AF-M2642 was not diminished the maximal binding signal was reduced by 2-fold (pH 7.4 A450=3.4 to pH 6.0 A450=1.7). Variants AF-M2632 (open circles), AF-M2633 (open squares), and AF-M2651 (closed squares) did not display saturable binding following a pH 6.0 wash step. Consequently, the EC50 values for these variants may be underestimated or were not calculated (AF-M2651). Variant AF-M2637 (inverted open triangles) displayed diminished binding both in EC50 (right shift) and the maximal signal was reduced following the pH 6.0 wash step.

The primary screen binding data for all variants is summarized in Table 3. Eighteen variants were eliminated from further consideration; fifteen due to weak binding following pH 7.4 washes and three that did not display a significant differential in dissociation rate between a pH 6.0 wash versus a pH 7.4 wash.

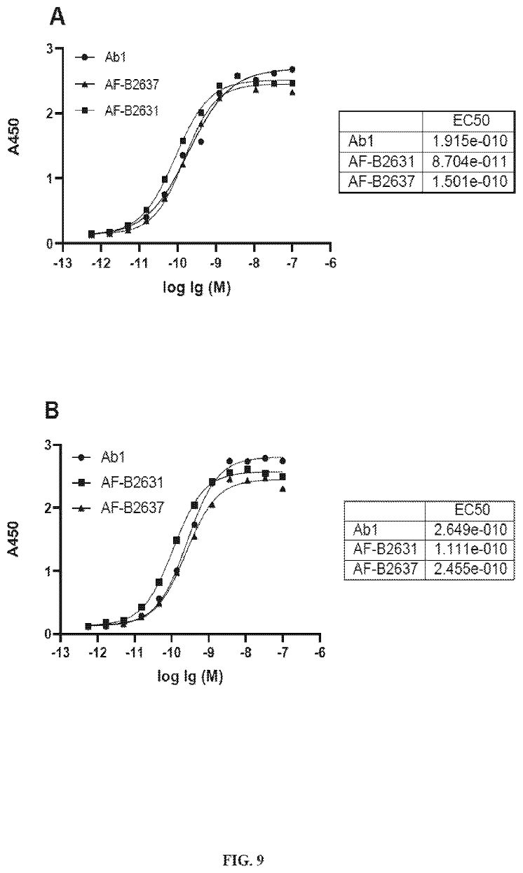

To examine the potential effect of avidity on screening pH switch variants, both the monovalent and bivalent formats of certain variants, including VH1 (AF-M2631 and AF-B2631) and VL4 (AF-M2637 and AF-B2637) were characterized. Ab1 and AF-M2630 (monovalent Ab1) were included as controls. Following pH 7.4 washes AF-M2631 bound with high affinity, equivalent to Ab1 (FIG. 5A). This was surprising because the monovalent format of Ab1, AF-M2630, displayed somewhat weaker binding than Ab1 (FIG. 2 and FIG. 5). These data are consistent with the pH switch mutation of AF-M2631 enhancing the affinity of its interaction with TNFα at pH 7.4. Following a pH 6.0 wash the EC50 of AF-M2631 increased by 30-fold while the EC50 of AF-M2637 increased by 15-fold (FIG. 5). Furthermore, the maximal binding of both variants was diminished by ˜2-fold. By contrast, the binding EC50 and maximal binding of the bivalent form of both variants was relatively unchanged following a pH 6.0 wash (FIG. 9). Specifically, the EC50 of AF-B2631 increased by 1.3-fold and the EC50 of AF-B2637 increased by 1.6-fold. These data highlight the importance of the valency of the format on the strength of activity of the pH switch.

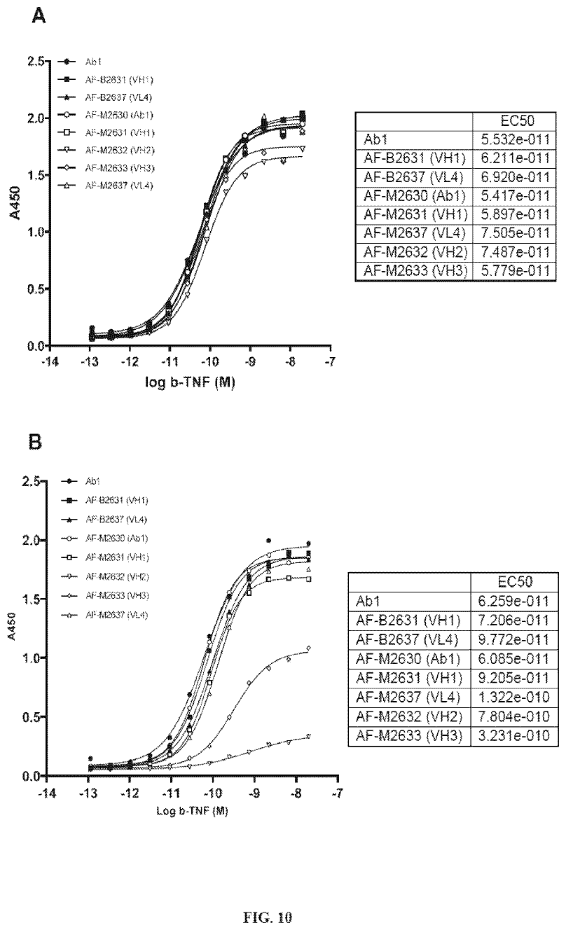

Additional experiments were performed to understand the potential impact of avidity on the activity of the pH switch. The binding of certain antibodies was characterized using a different assay format in which the antibodies were immobilized through the Fc and binding of soluble biotinylated human TNFα was assessed following a prolonged wash step at either pH 7.4 or pH 6.0. This assay format potentially mimics the capture of IC by FcR and FcRn. The pH switch of AF-M2632 (VH2) and AF-M2633 (VH3) was more effective than others, including AF-M2631 (VH1) and AF-M2637 (VL4) when the antibody is clustered in a manner that enables avidity (FIG. 10). In particular, we noted the diminishment of the pH switch activity of AF-M2637 in this assay format as opposed to the ELISA format in which TNFα was immobilized and the antibody was in solution (FIG. 5). The binding EC50s of AF-M2633 (VH3) and AF-M2632 (VH2) were increased by 5- to 10-fold, respectively and the maximal binding was diminished. The maximal binding of AF-M2632 was decreased significantly following the pH 6.0 wash (FIG. 10). These data are consistent with the previous data demonstrating the impact of multivalency on pH switch activity. Moreover, these data demonstrate that close clustering of monovalent constructs, such as that which may occur in endosomes, may also promote an avidity effect that diminishes the activity of pH switches.

### Example 6. Binding Kinetics of Ab1 and Variants-Bio-Layer Interferometry

The binding kinetics of pH switch variants at pH 7.4 or the dissociation kinetics at pH 6.0 were characterized using bio-layer interferometry (BLI). The kinetics of certain variants were characterized in both monovalent and bivalent formats to determine the impact of avidity on the pH switch activity, as it has previously been reported that the multivalent interaction of bivalent IgG can counteract pH sensitivity (Gera et al., supra; Schröter et al., supra; Traxlmayr et al., supra). Optimal candidates preserve high affinity binding of TNFα at pH 7.4 (EC50<100 nM), while displaying a >10-fold increase in dissociation rate at pH 6.0 relative to Ab1. Retention of high affinity at pH 7.4 may be important to enable the monovalent format to bind and neutralize both soluble (serum) TNFα and mTNFα in the absence of avidity. The high affinity interaction at pH 7.4 is expected to enable the internalization and targeted lysosomal degradation of TNFα (both IC and mTNFα) while the increase in dissociation rate at pH 6.0 will allow the antibody to release from the TNFα in the endocytic pathway and recycle back to the cell surface via interaction with FcRn.

### Materials and Methods

### Biotinylation of Human TNFα

To biotinylate human TNFα (Genscript), 200 μg was resuspended in 400 μL of water (500 μg/mL final concentration). Immediately prior to use, 1 mg of EZ-Link Sulfo-NHS-LC-Biotin (ThermoFisher Scientific) was resuspended in 180 μL of water, and subsequently 1.9 μL of the diluted EZ-Link Sulfo-NHS-LC-Biotin was added to 400 μL of the human TNFα to label at a molar ratio of 5:1 (biotin:TNFα). The reaction proceeded for 30 min at 25° C. at which time it was quenched by the addition of 2 μL of 1 M Tris, pH 7.4. The reaction was diluted to 500 μL with PBS and excess unreacted biotin was removed by placing the material on a Zeba 5 mL spin desalting column, 7K MWCO (ThermoFisher Scientific) that had been prepped per manufacturer's directions (4×2.5 mL washes with PBS). Biotinylated human TNFα was collected by centrifugation at 1000×g for 2 min and the final collected volume was measured in order to estimate the concentration of biotinylated TNFα (assuming 100% recovery).

### Bio-Layer Interferometry

BLI-based measurements addressing the pH-sensitive antigen binding were obtained using the Octet Red96 system (Forte-Bio) at 28° C., with orbital sensor agitation at 1000 rpm in 200 μL volume. Following a pre-hydration step for 10 min in PBS, 0.1% BSA, pH 7.4 (Octet Assay Diluent) and a 30 s sensor check, streptavidin biosensors were loaded with biotinylated human TNFα (3 μg/mL in PBS, 0.1% BSA, pH 7.4) for 700 s. After the loading step, the sensors were rinsed in PBS, 0.1% BSA, pH 7.4 for 480 s (baseline step). Association of antibodies at four concentrations (1, 3, 10, and 30 nM) was monitored for 600 s followed by the dissociation step with either PBS, 0.1% BSA, pH 7.4 or with PBS, 0.1% BSA, pH 6.0 for 600 s.

The association and dissociation sensorgrams were fit using a 1:1 Langmuir curve fit (Octet Analysis Software v. 8.2). Binding curves of antibodies generated by association and dissociation at pH 7.4 were analyzed by global full fitting, while binding curves generated by dissociation at pH 6.0 were analyzed using local partial fitting.

### Results

TNFα protein is a homotrimer, and thus enables multivalent interactions if the antibodies are immobilized. Consequently, the assay was configured with biotinylated human TNFα immobilized (ligand) and the antibody was loaded (analyte). Although bivalent antibodies may still display avidity in this assay format, it enabled accurate quantitation of the affinity of the monovalent variants.

The binding and dissociation of Ab1, AF-M2630 (monovalent Ab1) and five variants were characterized in the initial study. As expected, the bivalent nature of Ab1 combined with the assay format and the density of immobilized TNFα resulted in little dissociation of the antibody over the duration of the 600 s dissociation step. By contrast, when the monovalent format of Ab1 was tested in the same format, dissociation of the antibody was readily observed at the highest concentration during the 600 s wash step (data not shown).

Although the high avidity of Ab1 precluded an accurate affinity determination, the affinities of all the monovalent variants, including AF-M2630 (monovalent Ab1), were determined (Table 4). The monovalent pH switch variants displayed a range of affinities. AF-M2633 (monovalent VH3) and AF-M2637 (monovalent VL4) displayed higher affinities than the combinatorial variant, AF-M2652 (monovalent VH3/VL4). The five variants characterized in this study all displayed lower affinity than monovalent Ab1 (1.2 nM), ranging from 7- to 150-fold lower (Table 4). The rank order of the affinities of the variants were consistent with the rank order of affinities determined by ELISA (Example 5).

TABLE 4
Antibody kinetics at pH 7.4

| Construct | | K_{D} | kₒₙ | k_{dis} | | Relative |
|---|---|---|---|---|---|---|
| (Description) | Valency | (M) | (M⁻¹s⁻¹) | (s⁻¹) | R² | K_{D} |
| AF-B2630 | 2 | <1.0E−12 | 1.94E+05 | <1.0E−07 | 1.00 | |
| (Ab1) | | | | | | |
| AF-M2630 | 1 | 1.18E−09 | 7.30E+04 | 8.64E−05 | 1.00 | 1.0 |
| (mono-Ab1) | | | | | | |
| AF-M2632 | 1 | 4.32E−08 | 3.74E+04 | 1.61E−03 | 1.00 | 36.6 |
| (mono-VH2) | | | | | | |
| AF-M2633 | 1 | 1.91E−08 | 9.70E+04 | 1.86E−03 | 1.00 | 16.2 |
| (mono-VH3) | | | | | | |
| AF-M2651 | 1 | 2.76E−08 | 7.37E+04 | 2.04E−03 | 1.00 | 23.4 |
| (mono-VH3/VL3) | | | | | | |
| AF-M2637 | 1 | 8.44E−09 | 5.41E+04 | 4.56E−04 | 1.00 | 7.2 |
| (mono-VL4) | | | | | | |
| AF-M2652 | 1 | 1.80E−07 | 1.24E+05 | 2.22E−02 | 0.99 | 152.5 |
| (mono-VH3/VL4) | | | | | | |

Next, the variants were characterized for binding at pH 7.4 followed by dissociation at pH 6.0 and the data was analyzed using local partial fit (data not shown). AF-M2652 (VH3/VL4) bound weakly and AF-M2630 (monovalent Ab1) displayed the slowest dissociation rate. All pH switch variants characterized in this study displayed faster dissociation rates at pH 6.0 than did AF-M2630 (monovalent Ab1). The dissociation curves of pH switch variants AF-M2632, AF-M2633, and AF-M2651 all returned to baseline within the first 200 s of the dissociation wash step (data not shown). The k_{dis} of the variants relative to AF-M2630 (monovalent Ab1) was quantitated. For this calculation, the relative k_{dis} (Table 5, last column) was defined as the ratio of the kais as determined by local partial fitting of the variants compared to the k_{dis} of AF-M2630 (monovalent Ab1) at pH 7.4, as determined by global fitting. Ignoring AF-M2652 because the binding was so poor, the variants displayed 82- to 479-fold faster dissociation rates than AF-M2630 (monovalent Ab1) (Table 5).

TABLE 5
Monovalent antibody dissociation rates at pH 6.0

| Construct | Concentration | k_{dis} | | Average | Relative |
|---|---|---|---|---|---|
| (Description) | (nM) | (s⁻¹) | R² | k_{dis} (s⁻¹) | k_{dis} |
| AF-M2632 | 30 | 4.09E−02 | 1.00 | 4.46E−02 | 479 |
| (mono-VH2) | 10 | 4.60E−02 | 1.00 | | |
| | 3 | 5.12E−02 | 1.00 | | |
| | 1 | 4.01E−02 | 0.98 | | |
| AF-M2633 | 30 | 2.49E−02 | 1.00 | 2.44E−02 | 262 |
| (mono-VH3) | 10 | 2.37E−02 | 1.00 | | |
| | 3 | 2.44E−02 | 1.00 | | |
| | 1 | 2.44E−02 | 1.00 | | |
| AF-M2651 | 30 | 2.72E−02 | 1.00 | 2.74E−02 | 295 |
| (mono- | 10 | 2.60E−02 | 1.00 | | |
| VH3/VL3) | 3 | 2.89E−02 | 1.00 | | |
| | 1 | 2.76E−02 | 1.00 | | |
| AF-M2637 | 30 | 7.58E−03 | 1.00 | 7.63E−03 | 82 |
| (mono-VL4) | 10 | 7.31E−03 | 1.00 | | |
| | 3 | 7.78E−03 | 1.00 | | |
| | 1 | 7.84E−03 | 1.00 | | |
| AF-M2652 | 30 | 4.03E−01 | 0.97 | 3.05E−01 | 3281 |
| (mono- | 10 | 2.99E−01 | 0.95 | | |
| VH3/VL4) | 3 | 5.17E−01 | 0.86 | | |
| | 1 | 2.99E−03 | 0.76 | | |

A follow up study was performed in which pH switch variants AF-2631 (VH1) and AF-2637 (VL4) were compared to Ab1, in both monovalent and bivalent formats (Table 6). First, AF-M2637 and AF-M2631 proteins were further purified using protein L affinity chromatography. As expected, the bivalent format of Ab1 and the pH switch variant bound tightly and little dissociation was observed. Both AF-M2631 (2.7 nM) and AF-M2637 (4.1 nM) bound TNFα with high affinity. The kinetics of AF-M2630 (K_{D}=1.12 nM) were similar to the first study (K_{D}=1.18 nM). Following the additional purification of AF-M2637, its affinity was determined to be approximately 2-fold higher (K_{D}=4.1 nM) than the first determination with the less purified material (K_{D}=8.4 nM). Additionally, purified AF-M2631 was stored at both 4° C. and −20° C. for 6 months. The binding kinetics of AF-M2631 were similar, regardless of storage conditions (Table 6, K_{D}=2.66 nM vs 2.25 nM).

TABLE 6
Antibody kinetics at pH 7.4

| Construct | | K_{D} | kₒₙ | k_{dis} | | Relative |
|---|---|---|---|---|---|---|
| (Description) | Valency | (M) | (M⁻¹s⁻¹) | (s⁻¹) | R² | K_{D} |
| AF-B2630 | 2 | <1.0E−12 | 1.94E+05 | <1.0E−07 | 1.00 | |
| (Ab1) | | | | | | |
| AF-B2631 | 2 | <1.0E−12 | 4.14E+05 | <1.0E−07 | 1.00 | |
| (VH1) | | | | | | |
| AF-B2637 | 2 | <1.0E−12 | 1.90E+05 | <1.0E−07 | 1.00 | |
| (VL4) | | | | | | |
| AF-M2630 | 1 | 1.12E−09 | 8.33E+04 | 9.31E−05 | 1.00 | 1.0 |
| (mono-Ab1) | | | | | | |
| AF-M2631 | 1 | 2.66E−09 | 2.07E+05 | 5.51E−04 | 1.00 | 2.4 |
| (mono-VH1) | | | | | | |
| 4° C. storage | | | | | | |
| AF-M2631 | 1 | 2.25E−09 | 2.75E+05 | 3.61E−04 | 0.99 | 2.0 |
| (mono-VH1) | | | | | | |
| −20° C. storage | | | | | | |
| AF-M2637 | 1 | 4.06E−09 | 8.90E+04 | 4.56E−04 | 1.00 | 3.6 |
| (mono-VL4) | | | | | | |

The variants were characterized for binding at pH 7.4 followed by dissociation at pH 6.0 and the data was analyzed using local partial fit. The bivalent form of all antibodies bound tightly with little dissociation observed during the 600 s wash step, consistent with the avidity effect observed by ELISA. The dissociation rates of AF-M2637 and AF-M2631 were 32- and 51-fold faster, respectively, than AF-M2630 (Table 7). Characterization of the activity of the pH switch by BLI confirms the importance of both the assay format and the construct valency in identifying an active pH switch. Multiple monovalent variants (AF-M2631, AF-M2632, AF-M2633, AF-M2637, and AF-M2651) displayed affinities <50 nM at pH 7.4 with dissociation rates enhanced by >30-fold at pH 6.0.

TABLE 7
Monovalent antibody dissociation rates at pH 6.0

| Construct | Concentration | k_{dis} | | Average | Relative |
|---|---|---|---|---|---|
| (Description) | (nM) | (s⁻¹) | R² | k_{dis} (s⁻¹) | k_{dis} |
| AF-M2631 | 30 | 5.81E−03 | 1.00 | 4.71E−03 | 51 |
| (mono-VH1) | 10 | 5.10E−03 | 1.00 | | |
| 4° C. storage | 3 | 3.97E−03 | 1.00 | | |
| | 1 | 3.94E−03 | 1.00 | | |
| AF-M2631 | 30 | 6.12E−03 | 1.00 | 4.91E−03 | 53 |
| (mono-VH1) | 10 | 5.50E−03 | 1.00 | | |
| −20° C. storage | 3 | 4.40E−03 | 1.00 | | |
| | 1 | 3.61E−03 | 1.00 | | |
| AF-M2637 | 30 | 3.42E−03 | 1.00 | 2.96E−03 | 32 |
| (mono-VL4) | 10 | 2.87E−03 | 1.00 | | |
| | 3 | 2.60E−03 | 1.00 | | |
| | 1 | ND | 1.00 | | |

### Example 7. Binding of Ab1 and Variants to Membrane-Associated TNFα

There are potentially two distinct TNFα-dependent pathways to traffic anti-TNFα antibodies to lysosomes, wherein the antibodies may be processed and peptides presented on MHC class I and class II molecules. In one, antibodies bind soluble TNFα in the periphery and form IC with TNFα homotrimers. The IC bind FcR and FcRn, then internalize and traffic to the lysosomes for degradation. Alternatively, antibodies may bind membrane-associated TNFα (mTNFα), internalize, and traffic to the lysosomes for degradation. FcR, FcRn, and mTNFα are all present on professional antigen presenting cells, such as dendritic cells. A single APC can simultaneously cross-present antigens via MHC class I to CD8⁺ T cells and present antigens via MHC class II to CD4⁺ T cells which provide the T cell help necessary for initiating CD8⁺ T cell effector responses (reviewed in Baker et al., supra).

The monovalent antibody format cannot cross-link TNFα trimers, and thus prevents the formation of large IC (IC containing two or more TNFα trimers and greater than three antibodies). Monomeric antibodies may form small IC (1-3 antibodies per TNFα homotrimer), but small IC do not traffic to lysosomes to the same extent as large IC (Ortiz et al., Science Translational Medicine (2016) 8:365ra158). Nonetheless, monomeric variants may still bind mTNFα, internalize, and traffic to the lysosomes (Deora et al., supra).

The binding of mTNFα is postulated to be important for mediating the efficacy of Ab1 in IBD (Atreya et al., Gastroenterology (2011) 141:2026-38; reviewed in Levin et al., J. Crohn's and Colitis (2016) doi: 10.1093/ecco-jcc/jw053: 989-97). Ab1 reportedly binds mTNFα (K_{D}=2640 pM) less tightly than soluble TNFα (K_{D}=127 pM; Shealy et al., mAbs (2010) 2:428-39). Because the affinity of Ab1 is weaker for mTNFα than it is for soluble TNFα, avidity may be critical for its binding. The objective of the studies described in this example are twofold: (1) to characterize the impact of the monovalent format on binding to mTNFα and (2) to examine the impact of the pH switch mutations on binding to mTNFα.

### Materials and Methods

Construction of a Stable mTNFα-Expressing CHO Cell Line

DNA encoding a membrane-bound mutant TNFα, with amino acids 77 through 88 removed (Arora et al., Cytokine (2009) 45:124-31), was synthesized (amino acid sequence: SEQ ID NO: 81; nucleotide sequence: SEQ ID NO: 80). A stable CHO K-1 cell pool was generated by Genscript. Subsequently, single cell cloning was performed by flow cytometry using a murine anti-human TNFα membrane form fluorescein-conjugated monoclonal antibody (R & D Systems) to sort cells based on mTNFα expression. The mTNFα-expressing CHO K-1 cell line clone 3 was cultured in Ham's F-12K (Kaighn's) medium with 10% heat inactivated fetal bovine serum (Gibco) and 15 mg/mL puromycin (Pepro Tech Inc.).

### Flow Cytometry

The mTNFα-expressing CHO cell line was split 2-fold and plated in a T-75 flask one day prior to the experiment. The following day the media was removed, the cells were rinsed once with 5 mL DPBS, and the cells were detached by the addition of 2 mL of 0.25% Trypsin-EDTA for 10 min at 37° C. The released cells were collected by centrifugation at 500×g for 5 min. Next, the cells were resuspended at 5E06 cells/mL in 1 mL clear Ham's Nutrient Mixture F-12, GlutaminePlus, no phenol red (R&D Systems) with 10% FBS and 25 mL of FcX (BioLegend, 5 λL FcX per 1 E06 cells) and the cells were placed on ice for 1 h. Subsequently, 100 μL of cells (5E05 cells) was aliquoted and 1 μg of antibody variants (10 μg/mL final concentration), HUC2-MRVA negative control, murine IgG1, 2 μL kappa isotype-FITC control (BioLegend), or 5 μL of human TNFα membrane form fluorescein-conjugated antibody was added. The cells were incubated with antibody on ice for 1 h. The cells were washed two times, 1 mL each, with Ham's Nutrient Mixture F-12, GlutaminePlus, no phenol red with 10% FBS and 2.5 μL of FcX. Cells were collected between washes by centrifugation at 500×g for 5 min at 4° C. Following the final wash, cells were resuspended in 100 μL Ham's Nutrient Mixture F-12, GlutaminePlus, no phenol red with 10% FBS and 2.5 μL of FcX. To detect antibody binding, 10 μL of a 1:100 dilution of goat F(ab)₂ anti-human kappa-FITC (SouthernBiotech) was added and incubated for 1 h on ice. The cells were washed two times, 1 mL each, with Ham's Nutrient Mixture F-12, GlutaminePlus, no phenol red with 10% FBS and 2.5 μL of FcX. Cells were collected between washes by centrifugation at 500×g for 5 min at 4° C. Finally. the samples were resuspended in 300 μL Ham's Nutrient Mixture F-12, GlutaminePlus, no phenol red with 10% FBS and analyzed on an Attune NXT flow cytometer.

mTNFα-Expressing CHO Cell Line ELISA

One day prior to the experiment, mTNFα CHO cells were plated in a 96-well cell culture plate at 3.5 E04 cells/well in 100 μL of Ham's F-12K (Kaighn's) medium with 10% heat inactivated FBS and 15 μg/mL puromycin. Twenty hours later, the cells were transferred to an ice bath, the media was removed and replaced with 100 μL of ice-cold Ham's Nutrient Mixture F-12, GlutaminePlus, no phenol red with 10% FBS. Antibodies were diluted serially 2-fold beginning at 10 μg/mL in ice-cold Ham's Nutrient Mixture F-12, GlutaminePlus, no phenol red with 10% FBS and incubated with cells for 1 h on ice. The antibody was removed with gentle aspiration and the cells were washed three times with 200 μL of ice-cold DPBS. Next, 50 μL of goat anti-human IgG Fc-HRP (SouthernBiotech) diluted 1:4000 in Ham's Nutrient Mixture F-12, GlutaminePlus, no phenol red with 10% FBS was added and incubated with cells on ice for 1 h. The antibody was removed with gentle aspiration and the cells were washed three times with 200 μL of ice-cold DPBS. Finally, 50 μL of 1-Step Ultra-TMB-ELISA substrate solution was added and the reaction developed for 10 min at 25° C. The reaction was terminated by the addition of 50 μL of 2N H₂SO₄, the bottom of the plate was wiped to ensure removal of moisture, and the absorbance at 450 nm was quantitated using a Spectramax Plus 384 microplate reader. The cells were checked under the microscope to ensure minimal detachment had occurred during the manipulations. No significant loss of cells was observed in any experiment.

### Results

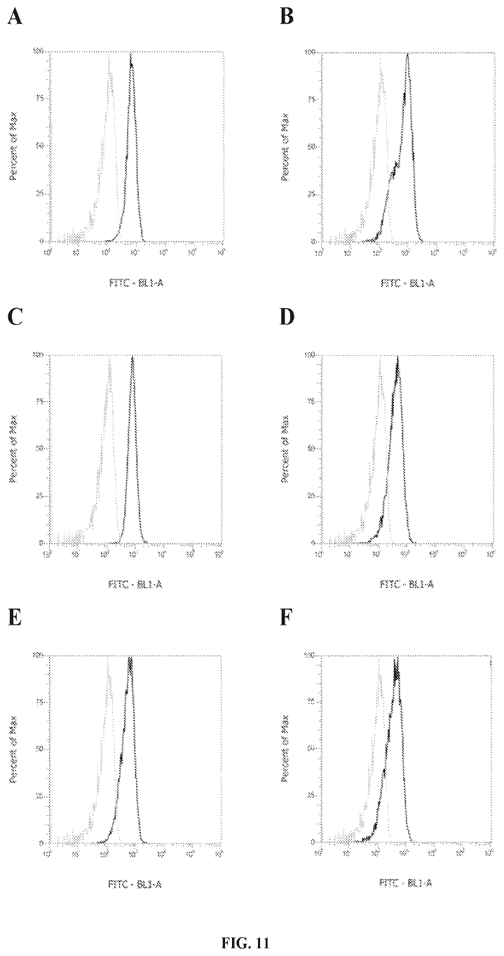

The binding of Ab1 and variants to the mTNFα-expressing CHO cell line clone 3 was initially evaluated by flow cytometry. All constructs bound the cells significantly better than the negative control antibody (FIG. 11, compare black versus gray histograms). The monovalent form of the two pH switch variants bound somewhat more weakly than the corresponding bivalent form (FIG. 11, compare right column versus left column; Table 8, MFI values). Surprisingly, monovalent Ab1 (AF-M2630) bound as strongly, or more strongly, than Ab1 (Table 8).

TABLE 8
Binding of Ab1 and variants to mTNFα-expressing CHO cell line

| Sample | Valency | MFI |
|---|---|---|
| Unstained | — | 94 |
| IgG1 isotype control for mTNF | 2 | 93 |
| Positive control (mTNF) | 2 | 805 |
| Negative control (HUC2-MRVA) | 2 | 102 |
| Ab1 | 2 | 681 |
| AF-M2630 (monovalent Ab1) | 1 | 780 |
| AF-B2631 (VH1) | 1 | 618 |
| AF-M2631 (monovalent VH1) | 1 | 431 |
| AF-B2637 (VL4) | 2 | 593 |
| AF-M2637 (monovalent VL4) | 1 | 417 |

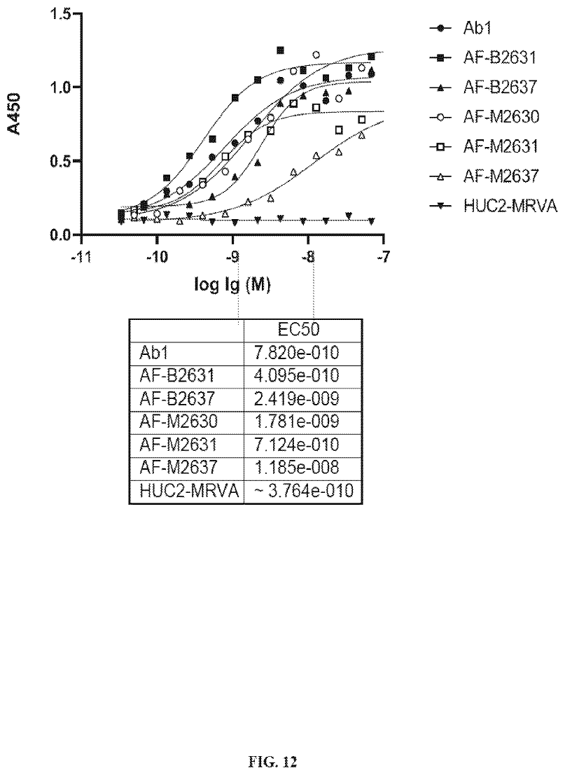

To enable a more quantitative comparison of the relative binding strength of the variants, a cell-based ELISA was developed. Briefly, mTNFα-expressing CHO cells were seeded in 96-well plates and after reaching confluence the cells were incubated with serial dilutions of antibodies. Prior to the addition of antibodies and in all subsequent steps, the cells were maintained on ice and all wash steps were performed with ice-cold buffer to prevent cell trafficking (internalization). Ab1 and the variants displayed dose-dependent binding to the live cells (FIG. 12) while no binding was observed with the irrelevant, isotype-matched control antibody (FIG. 12, inverted solid triangles). In addition, all variants, with the exception of AF-M2637 (FIG. 12, open triangles), displayed saturable binding.

For comparison, the flow cytometry experiments described above characterized the antibody binding at 10 μg/mL, which is slightly higher than the highest concentration of each construct in the ELISA-based assay. The relative signals of the variants to one another in the ELISA were consistent with the relative MFI values obtained by flow cytometry. For example, Ab1, AF-B2631, AF-B2637, and AF-M2630 generated the highest signals in both assays, while AF-M2631 and AF-M2637 generated the lowest. In summary, binding of Ab1 and all variants to membrane-associated TNFα was demonstrated by two different approaches using mTNFα-expressing CHO cells.

The monovalent formats of Ab1 and both pH switch variants bound mTNFα ˜2- to 5-fold weaker than the corresponding bivalent construct (Table 9). Avidity was slightly more pronounced with AF-B2637, likely because its affinity is lower than Ab1 and AF-B2631.

The binding EC50s of Ab1 and all variants to membrane-associated TNFα was compared to the binding EC50s obtained by ELISA using immobilized, recombinant TNFα (Table 9). The binding of Ab1, AF-M2630, AF-B2637, and AF-M2637 to mTNFα-expressing CHO cells was between 7- to 16-fold weaker than their binding to immobilized recombinant TNFα in an ELISA format. By contrast, the binding of the AF-B2631 and AF-M2631 was more similar (2-to 4-fold weaker binding to mTNFα) between the two TNFα assay formats (Table 9).

The concentration for half maximal binding (EC50) of both pH switch variants is below the serum antibody trough levels maintained in patients. The typical Ab1 plasma trough levels in responding Crohn's patients is 70-fold greater than the EC50 of AF-M2631 (0.7 nM) and is 7-fold greater than the EC50 of AF-M2637 (7.6 nM). Consequently, assuming the variants display pharmacokinetics similar to Ab1, saturation of binding to mTNFα may be possible with current dosing regimens.

TABLE 9
Ab1 and variant binding to recombinant TNFα (rTNFα) versus
mTNFα-expressing CHO cells

| | EC50 (M) | | | | |
|---|---|---|---|---|---|
| | Recombinant | | | | Ratio |
| Antibody | TNFα | AVG. | mTNFα | AVG. | (mTNFα/rTNFα) |
| Ab1 | 7.13E−11 | 9.24E−11 | 6.95E−10 | 6.32E−10 | 6.8 |
| | 2.19E−10 | | 7.82E−10 | | |
| | 1.16E−11 | | 4.18E−10 | | |
| | 9.76E−11 | | | | |
| | 6.31E−11 | | | | |
| AF-M2630 | 2.20E−10 | 1.60E−10 | 2.45E−09 | 1.66E−09 | 10.4 |
| | 1.24E−10 | | 1.78E−09 | | |
| | 1.35E−10 | | 7.57E−10 | | |
| AF-B2631 | 8.70E−11 | 8.70E−11 | 4.10E−10 | 3.42E−10 | 3.9 |
| | | | 3.43E−10 | | |
| | | | 2.74E−10 | | |
| AF-M2631 | 1.75E−10 | 3.11E−10 | 7.12E−10 | 6.72E−10 | 2.2 |
| | 4.47E−10 | | 6.31E−10 | | |
| AF-B2637 | 1.50E−10 | 1.50E−10 | 2.42E−09 | 1.55E−09 | 10.3 |
| | | | 1.49E−09 | | |
| | | | 7.39E−10 | | |
| AF-M2637 | 5.22E−10 | 4.64E−10 | 1.01E−08 | 7.58E−09 | 16.4 |
| | 3.22E−10 | | 1.19E−08 | | |
| | 5.47E−10 | | 8.01E−10 | | |

### Example 8. Cell Based Potency Assays with Human TNFα

The ability of Ab1 and variants to inhibit the binding of soluble human TNFα to TNFR was characterized using cell-based potency assays. Two reporter cell lines were employed: (1) the THP1-Blue™ NF-κB monocyte cell line that expresses TNFR, FcR, and mTNFα and FcRn under certain conditions, all of which may play a role in trafficking of TNFα antibodies and TNFα/Ig immune complexes and (2) the HEK-Blue™ cell line that is more sensitive to TNFα stimulation than the THP1-Blue™ cell line.

### Materials and Methods

Human TNFα Dose Response with HEK-Blue™ TNFα and THP1-Blue™ Cells

HEK-Blue™ TNFα cells were maintained in DMEM, 4.5 g/l glucose, 2 mM L-glutamine, 10% (v/v) heat-inactivated fetal bovine serum, 100 U/mL penicillin, 100 μg/mL streptomycin, 100 μg/mL Normocin™. THP1 Blue™ cells were maintained in RPMI 1640 media with 10% heat inactivated FBS. One day prior to the experiment, HEK-Blue cells were resuspended at 2.8E05 cells/mL and 180 μL (˜5E04 cells) was aliquoted to each well of a 96-well cell culture plate while THP1 Blue cells were resuspended at 1E06 cells/mL and 100 μL (1E05 cells) was aliquoted to each well of a 96-well cell culture plate. Next, 20 μL of human TNFα (Genscript, MW 52.2 kDa) was added at 2 ng/mL final concentration and was diluted serially, 2-fold. At the conclusion of the incubation, 20 μL of culture supernatant was added to 180 μL of QUANTI-Blue™ Solution (prepared per manufacturer's instructions) in a Corning 3797 96-well microplate and was incubated at 37° C. for <1 h. Expression of the reporter protein SEAP was quantitated at 650 nm using a spectrophotometer.

### THP-1 Potency Assay

THP1-Blue™ NF-κB cells (InvivoGen) were maintained in RPMI 1640 media with 10% heat inactivated FBS (Gibco). One day prior to the experiment, the cells were resuspended at 1E06 cells/mL and 100 μL (1E05 cells) was aliquoted to each well of a 96-well cell culture plate. Separately, antibodies were serially diluted 5-fold, beginning at 3.6 μM, mixed with 60 pM TNFα (Genscript) in a 96-well microplate and incubated 1 hr at 25° C. Subsequently, 20 μL of the dilution series (10 pM TNFα final concentration) was added to the cells and incubated for 20-24 h at 37° C. in 5% CO₂. At the conclusion of the incubation, 20 μL of culture supernatant was added to 180 μL of QUANTI-Blue™ Solution (InvivoGen) in a separate microplate and was incubated at 37° C. for less than 1 h. Expression of the reporter protein SEAP was quantitated at 650 nm using a spectrophotometer.

### HEK-Blue™ TNFα Potency Assay

HEK-Blue™ TNFα cells were maintained in DMEM, 4.5 g/l glucose, 2 mM L-glutamine, 10% (v/v) heat-inactivated fetal bovine serum, 100 U/mL penicillin, 100 μg/mL streptomycin, 100 μg/mL Normocin™. One day prior to the experiment, the cells were resuspended at 2.8E05 cells/mL and 180 μL (˜5E04 cells) was aliquoted to each well of a 96-well cell culture plate. Separately, antibodies were serially diluted 5-fold, beginning at 3 μM, mixed with 50 pM TNFα (Genscript) in a Corning 3797 96-well microplate and incubated 1 h at 25° C. Subsequently, 20 μL of the dilution series (5 pM TNFα final concentration) was added to the cells and incubated for 20-24 h at 37° C. in 5% CO₂. At the conclusion of the incubation, 20 μL of culture supernatant was added to 180 μL of QUANTI-Blue™ Solution (prepared per manufacturer's instructions) in a Corning 3797 96-well microplate and was incubated at 37° C. for <1 h. Expression of the reporter protein SEAP was quantitated at 650 nm using a spectrophotometer.

### Results

### Recombinant Human TNFα is a Potent Stimulator of HEK-Blue™ TNFα Cells.

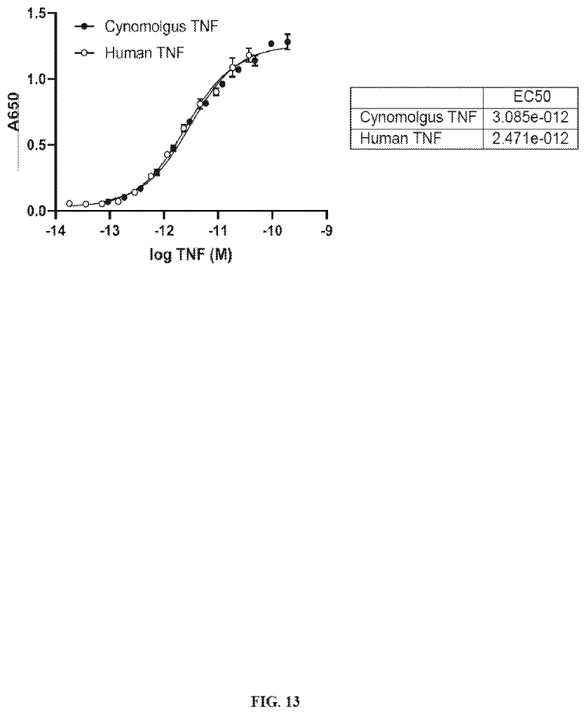

The HEK-Blue™ TNFα cell line responds to human TNFα by monitoring the activation of the AP-1/NF-κB pathway. The cells were derived from the human embryonic kidney 293 cell line by stable transfection with a secreted alkaline phosphatase (SEAP) reporter gene under the control of the IFN-β minimal promoter fused to five AP-1 and five NF-κB binding sites. Stimulation of the cells with TNFα leads to activation of AP1/NF-κB and the subsequent expression of SEAP (InvivoGen product materials). To gauge the sensitivity of the cell line to TNFα stimulation, the cells were treated with varying concentrations of human TNFα. The cells responded to human TNFα in a dose-dependent and saturable manner (FIG. 13). The EC50 for stimulation of the cells with human TNFα was 2.5 pM.

Stimulation of HEK-Blue Cells with Human TNFα is Inhibited by Ab1 and pH Switch Variants.

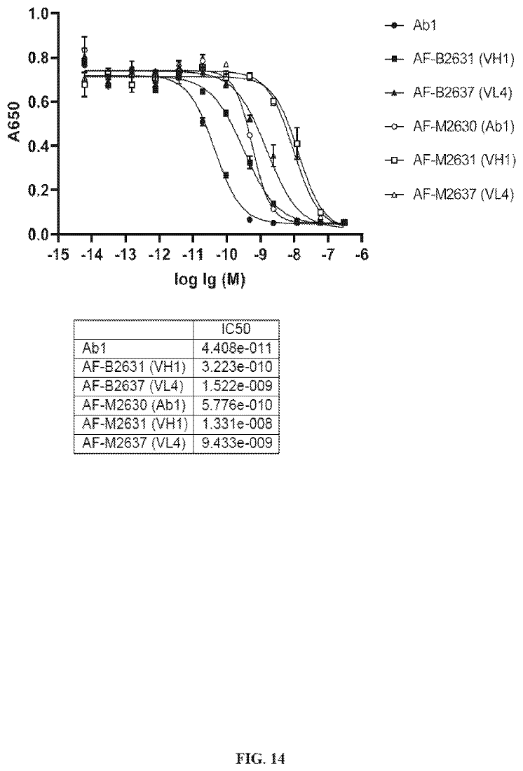

The ability of Ab1 and variants to inhibit the TNFα stimulation of the cells was evaluated. The antibodies were titrated with 5 pM human TNFα and the mixture was incubated with the cells for 20 h. Ab1 and the variants displayed ˜300-fold range of potencies against soluble human TNFα (FIG. 14). Ab1 (closed circles) was the most potent (IC₅₀₌₄₄ pM) while the monovalent pH switch variant AF-M2631 (open squares) was the least potent (IC₅₀₌₁₃ nM). The bivalent form of each variant (FIG. 14, closed symbols) was more potent than the corresponding monovalent format (FIG. 14, open symbols) for all three variants.

### TNFα Stimulation of THP1 Monocytes is Inhibited by Ab1 and pH Switch Variants

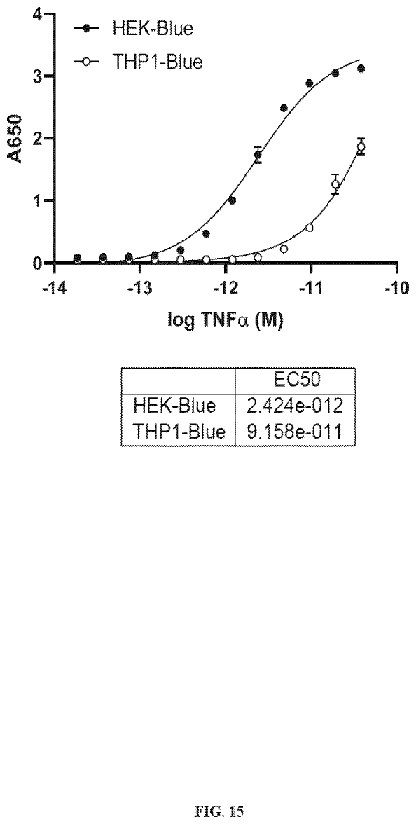

Although the HEK-Blue cells provided a sensitive assay for characterizing the relative potencies of the variants, we were interested in exploring the potencies on a potentially more relevant cell line. Monocytes and the monocyte cell line THP1 express TNFR, FcR (Fleit et al., J Leuk Biol. (1991) 49:556-65), and under certain circumstances, TNFα (Moreire-Tabaka et al., PLoS ONE (2012) 7: e34184. Doi: 10.1371/journal.pone.0034184 and FcRn (Zhu et al., J Immunol. (2001) 166:3266-76). Similar to the HEK-Blue™ TNFα cell line, THP1-Blue™ NF-κB cells were engineered to monitor the NF-κB signal transduction pathway by stable integration of an NF-κB-inducible SEAP promoter construct. Consequently, the cells allow monitoring of NF-κB activation by quantitating SEAP activity in the cell culture supernatant. To gauge the TNFα sensitivity of the THP1-Blue™ NF-κB cell line relative to the HEK-Blue™ TNFα cells, both cell lines were treated with varying concentrations of human TNFα. The THP1-Blue cells responded to human TNFα but were much less sensitive than the HEK-Blue cells (FIG. 15). While the HEK-Blue response was both dose-dependent and saturable (EC₅₀=2.4 pM), the THP1-Blue response was not saturable in the concentration range tested (EC₅₀>92 pM). Consequently, the EC₅₀ determination for THP1-Blue cells is likely an underestimation.

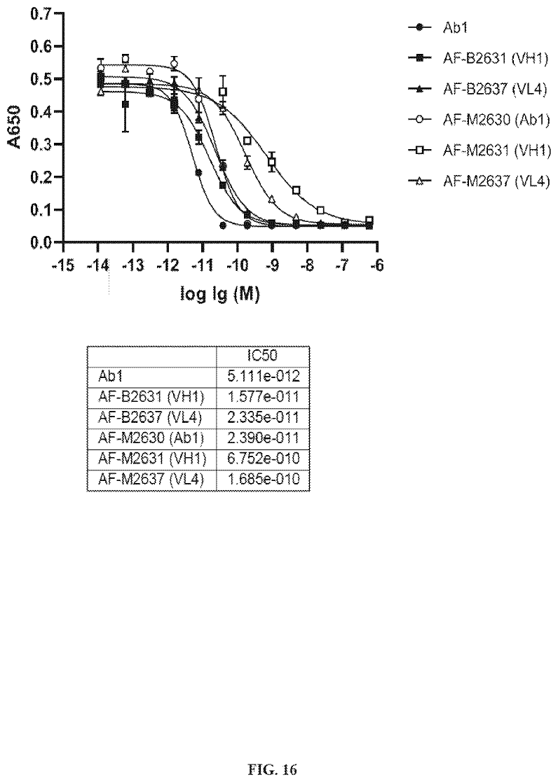

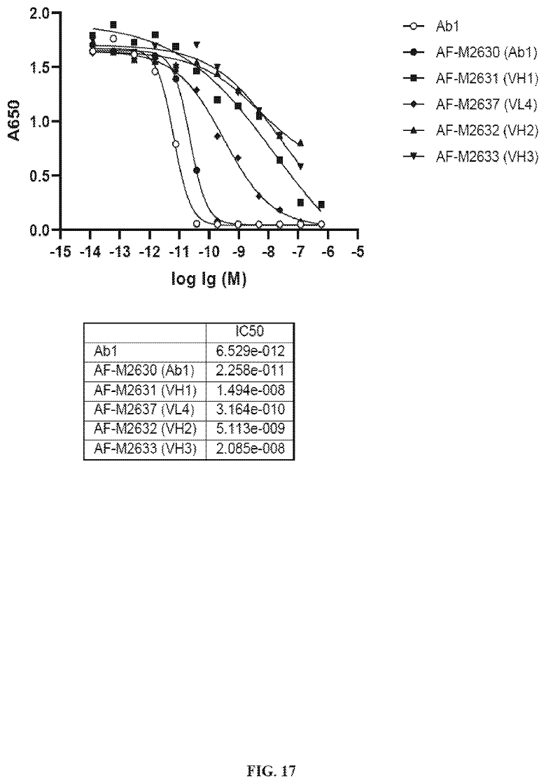

Nonetheless, the ability of Ab1 and variants to inhibit TNFα stimulation of the THP1-Blue cells was evaluated. The antibodies were titrated with 10 pM human TNFα and the mixture was incubated with the cells for 20 h. In the first experiment, Ab1 and the variants displayed a 130-fold range of potencies (FIG. 16). Ab1 (closed circles) was the most potent (IC₅₀=5.1 pM), while the monovalent pH switch variant AF-M2631 (open squares) was the least potent (IC₅₀=0.7 nM). Similar to the HEK-Blue assay, the bivalent form of each variant was more potent than the corresponding monovalent format for all three variants. Subsequently, additional variants were tested and Ab1 and certain other variants displayed a 780- to 3200-fold range of potencies (FIG. 17).

The THP1-Blue cell line was less sensitive to TNFα than the HEK-Blue reporter line (EC₅₀>100 pM vs 2.4 pM) and all the variants tested with both cell lines displayed at least 25-fold greater potency against TNFα stimulation of THP1 cells than against the HEK-Blue reporter line. Regardless of which reporter cell line was used, the bivalent constructs were more potent than the corresponding monovalent constructs. Depending on the reporter cell line used, the IC₅₀ of the pH switch variants, AF-M2631 and AF-M2637 was 4- to 5-fold (HEK-Blue cells) or >70-fold (THP1-Blue cells) below the Ab1 mean plasma trough level at week 48 (7.62 mg/mL, or 51 nM) observed after dosing at 40 mg every other week (Bodini et al., Scand J Gastroenterol. (2016) 51:1081-6).

### Example 9. Characterization of Binding to Murine TNFα

Prior to performing in vivo studies, the binding of monovalent and bivalent formats of Ab1 and certain pH switch variant antibodies to murine TNFα was characterized by ELISA.

### Materials and Methods

### Antibody Binding to Murine TNFα ELISA

A 96-well plate was coated with 50 μL of 2 μg/mL goat anti-human Fc (SouthernBiotech) in PBS for 1 h at 25° C. Subsequently, the plate was washed once (all wash steps were performed with PBS, 0.05% Tween 20) and was then blocked with 100 μL of PBS, 1% (w/v) BSA (Fisher Scientific) for 30 min at 25° C. Next, 50 μL of 1 μg/mL test article antibody diluted in PBS, 1% (w/v) BSA was added and incubated for 1 h at 25° C. The plate was washed four times and 50 μL of biotinylated murine TNFα (Acro Biosystems) diluted serially, 3-fold in PBS, 1% (w/v) BSA was incubated for 1 h at 25° C. The plate was washed four times and 50 μL of neutravidin-HRP (ThermoFisher Scientific) diluted 1:2000 in PBS, 1% (w/v) BSA was added for 1 h at 25° C. Alternatively, for prolonged dissociation at pH 6.0 the plate was submerged in 500 mL of PBS, 0.05% Tween 20, pH 6.0 for 1 h at 25° C. prior to the addition of neutravidin-HRP. To ensure thorough washing, buffer was tapped out of the plate every 15 min. The plate was washed four times and was developed using 50 μL/well of Ultra-TMB-ELISA substrate (ThermoFisher Scientific). The reaction was terminated by the addition of 50 μL/well of 2N H₂SO₄ and the absorbance at 450 nm was quantitated using a Spectramax Plus 384 microplate reader (Molecular Devices). The absorbance of the greatest dilution sample of each variant was subtracted as background.

### Results

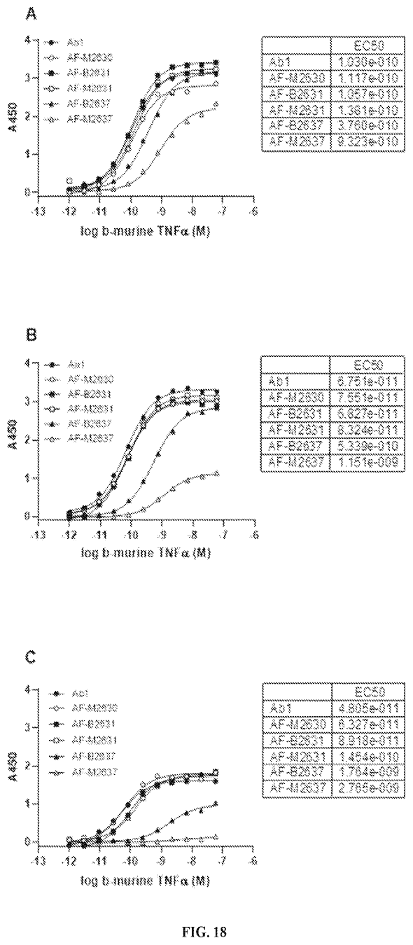

Ab1, AF-M2630 (monovalent Ab1), AF-B2631, AF-M2631, AF-B2637, and AF-M2637 were all characterized for binding to murine TNFα. First, the antibodies were captured with anti-human Fc antibody. Following titration of biotinylated mouse TNFα, the plates were washed, either at pH 7.4 or at pH 6.0, and subsequently, antibody binding was quantitated using neutravidin-HRP conjugate. All antibodies bound murine TNFα following a short pH 7.4 wash (FIG. 18A). The binding of the pH switch AF-M2637 was weakest (EC₅₀=0.9 nM) while the bivalent form of the same pH switch, AF-B2637, bound ˜2.5-fold more tightly (EC₅₀=0.4 nM). The binding of Ab1, AF-M2630, AF-B2631, and AF-M2631, were all very similar (EC₅₀=96-138 pM).

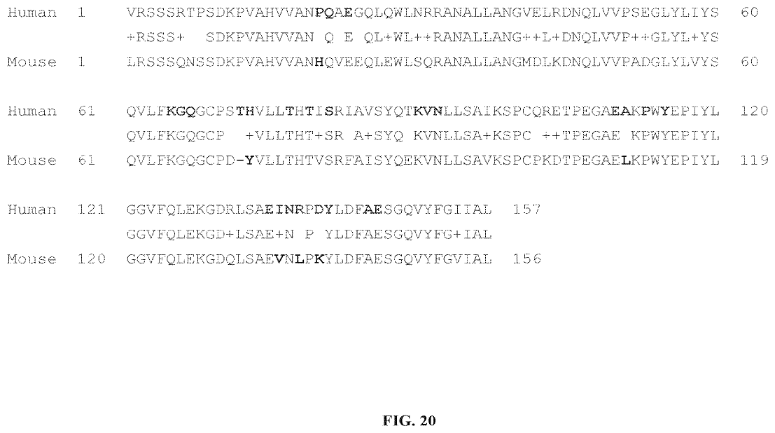

Next, the binding of all variants was characterized following prolonged washes at pH 7.4 and pH 6.0. The binding EC50 of all variants following the long pH 7.4 wash (FIG. 18B) were similar to the EC50 observed following the short pH 7.4, but the maximal binding signal of AF-M2637 was diminished ˜2-fold. As was observed with binding to human TNFα, Ab1 and monovalent Ab1 (AF-M2630) displayed similar EC50 following a long pH 6.0 wash (FIG. 18C) or a long pH 7.4 wash. The pH switch variant AF-M2631 and its corresponding bivalent form, AF-B2631, also did not demonstrate a significant reduction in EC50 following the pH 6.0 wash (FIG. 18C). Thus, the pH switch effect of AF-M2631 observed with human TNFα was not preserved with mouse TNFα. This was not entirely unexpected as the soluble extracellular domain of murine TNFα is only 79% identical (124/157 residues) to the soluble extra-cellular domain of human TNFα and it differs at 7/26 epitope residues identified by Hu et al., 2013 (FIG. 20). By contrast, following the pH 6.0 dissociation step the binding of AF-B2637 was diminished >3-fold and there was no detectable binding of AF-M2637 (FIG. 18C).

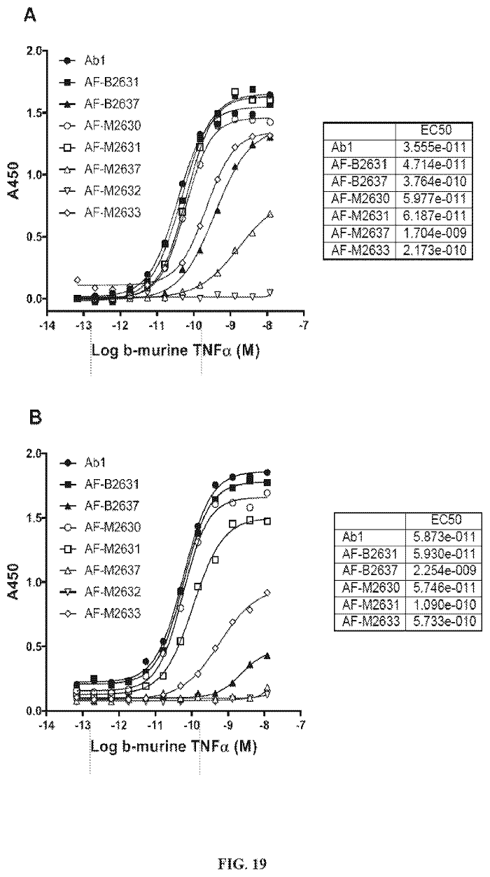

Additional pH switch variants (AF-M2632 and AF-M2633) were characterized for binding to murine TNFα. AF-M2633 bound with high affinity (EC50=2.17E-10) at pH 7.4, while no binding of AF-M2632 was observed (FIG. 19A). Following a prolonged pH 6.0 wash, AF-M2633 still bound murine TNFα with high affinity (FIG. 19B, EC50=5.73E-10). Thus, similar to AF-M2631, the pH switch effect of AF-M2633 observed with human TNFα was not preserved with mouse TNFα.

### Example 10. Anti-Drug Antibodies (ADA) are Formed Against Ab1, AF-M2630, AF-B2631, AF-M2631 and AF-B2637, but not Against the Monovalent pH Switch Variant AF-M2637

The immunogenicity of Ab1 and certain variants was characterized in mice. Previously, a robust anti-drug antibody (ADA) immune response against Ab1 has been observed following a single 4 mg/kg injection in mice (Arnoult et al., supra).

### Materials and Methods

### Murine Anti-Ab1 ELISA (ADA)

A 96-well plate was coated with 50 μL of 1 μg/mL of test article antibody in PBS for 1 h at 25° C. Subsequently, the plate was washed once (all wash steps were performed with PBS, 0.05% Tween 20) and was then blocked with 100 μL of PBS, 5% (w/v) dried milk for 1 h at 25° C. The plate was washed once and 50 μL of serum sample diluted 1:100 in PBS, 5% (w/v) dried milk was added for 1 h at 25° C. The plate was washed four times and 50 μL goat anti-mouse IgG, human adsorbed-HRP (SouthernBiotech) or goat anti-mouse IgM, human adsorbed-HRP (SouthernBiotech) diluted 1:4000 in PBS, 5% (w/v) dried milk was added and incubated for 1 h at 25′° C. The plate was washed four times and was developed using 50 μL/well of Ultra-TMB-ELISA substrate (ThermoFisher Scientific). The reaction was terminated by the addition of 50 μL/well of 2N H₂SO₄ and the absorbance at 450 nm was quantitated using a Spectramax Plus 384 microplate reader (Molecular Devices).

### Murine Immunogenicity Study

Experiments were performed by Explora BioLabs (San Diego, CA) and the study protocol and procedures involving the care and use of animals were approved by the Explora BioLabs' IACUC. Six-to ten-week old female C57BL/6 mice were obtained from Charles Rivers Laboratories. The animals were allowed to acclimate to the housing environment for at least three days prior to the initiation of the study and their general health was monitored prior to inclusion in the study. Body weights were measured during acclimation and the animals were randomized to obtain similar average body weight between treatment groups. Each treatment group contained six mice and serum was collected from three of the mice at each time point. Test articles were supplied at 0.8 mg/mL and were administered at 4 mg/kg via a single intravenous injection via the lateral tail vein on day 0. Blood samples were collected at pre-dose, and on days 1, 3, 6, 7, 9, 14 and 21. Blood samples were collected in serum separator tubes, allowed to clot at 25° C. for 20-30 min, and were processed to serum by centrifugation at 5000 rpm for 5 min. The samples were frozen and stored at −80° C.

### Results

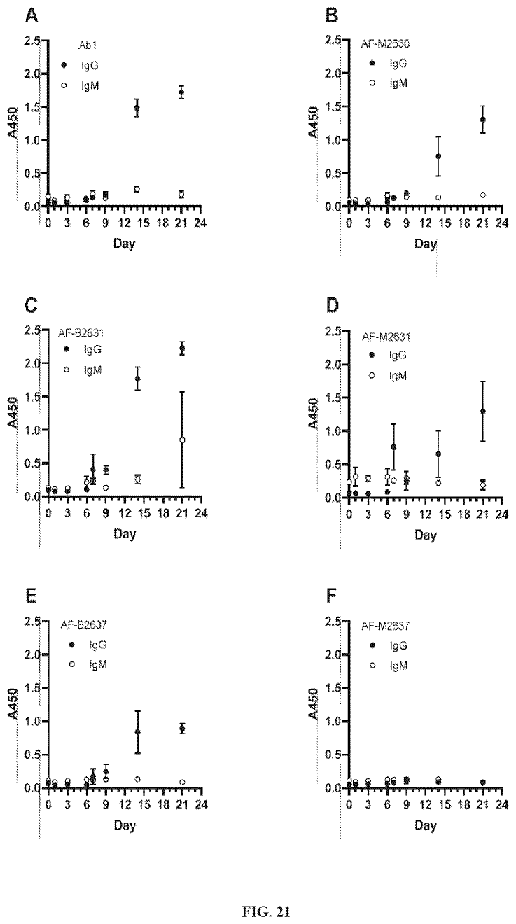

Mice were administered a single, intravenous 4 mg/kg dose of Ab1, AF-M2630, AF-B2631, AF-M2631, AF-B2637, and AF-M2637 and IgG and IgM anti-drug antibody (ADA) were monitored for 21 days. No detectable ADA (IgG or IgM) was observed over the 21 day duration of the experiment in the mice administered the monovalent pH switch variant AF-M2637 (FIG. 21F). By contrast, ADA (IgG) was detected by day 7 and increased significantly by day 14 in mice administered Ab1 and the other variants (FIG. 21A-E, AF-M2630, AF-B2631, AF-M2631 and AF-B2637). The increased IgG titer at day 14 in Ab1 (FIG. 21A) was greater than that observed at day 14 with AF-M2630 (FIG. 21B), AF-M2631 (FIG. 21D) or AF-B2637 (FIG. 21E). The IgG response was even more pronounced by day 21 in the Ab1 and AF-B2631 groups. The IgG response observed with AF-M2631 at day 7 and day 14 was driven predominantly by a large response from one of the three mice sampled on those two days. A modest IgM response was observed at day 14 in the Ab1 and AF-B2631 groups. Minimal IgM response was observed with AF-M2630, AF-M2631, AF-B2637, and AF-M2637, though we noted the assay background was higher in the AF-M2631 treatment group.

### Example 11. Monovalent pH Switch Variant AF-M2637 has a Slower Elimination Profile than Ab1

The immunogenicity of Ab1 and certain variants was characterized in mice. Previously, a robust anti-drug antibody (ADA) immune response against Ab1 has been observed following a single 4 mg/kg injection in mice (Arnoult et al., supra).

The pharmacokinetics of Ab1 and certain variants were characterized in mice. Previously, Ab1 has been observed to display an accelerated terminal elimination following a single 4 mg/kg injection in mice, consistent with an ADA response (Arnoult et al., supra).

### Materials and Methods

### Serum Ab1 ELISA (Pharmacokinetics)

A 96-well plate was coated with 50 μL of 1 μg/mL recombinant human TNFα (Genscript) in PBS for 1 h at 25° C. Subsequently, the plate was washed once (all wash steps were performed with PBS, 0.05% Tween 20) and was then blocked with 100 μL of PBS, 1% (w/v) BSA (Fisher Scientific) for 30 min at 25° C. The plate was washed once and 50 μL of serum sample diluted 1:10 and then serially three-fold in PBS, 1% (w/v) BSA was added for 1 h at 25° C. The plate was washed four times and 50 μL of goat anti-human kappa-HRP (SouthernBiotech) diluted 1:4000 in PBS, 1% (w/v) BSA was added for 1 h at 25° C. The plate was washed four times and was developed using 50 μL/well of Ultra-TMB-ELISA substrate (ThermoFisher Scientific). The reaction was terminated by the addition of 50 μL/well of 2N H₂SO₄ and the absorbance at 450 nm was quantitated using a Spectramax Plus 384 microplate reader (Molecular Devices). A standard curve was created for each test article, beginning at 6.5 ng/ml and diluting serially 3-fold in PBS containing 1% (w/v) BSA to 36.7 pg/mL. Serum antibody levels were determined as the average of all values in the linear range of the standard curve.

### Murine Pharmacokinetic Study

Experiments were performed by Explora BioLabs (San Diego, CA) and the study protocol and procedures involving the care and use of animals were approved by the Explora BioLabs' IACUC. Six-to ten-week old female C57BL/6 mice were obtained from Charles Rivers Laboratories. The animals were allowed to acclimate to the housing environment for at least three days prior to the initiation of the study and their general health was monitored prior to inclusion in the study. Body weights were measured during acclimation and the animals were randomized to obtain similar average body weight between treatment groups. Each treatment group contained six mice and serum was collected from three of the mice at each time point. Test articles were supplied at 0.8 mg/mL and were administered at 4 mg/kg via a single intravenous injection via the lateral tail vein on day 0. Blood samples were collected at pre-dose, and on days 1, 3, 6, 7, 9, 14 and 21. Blood samples were collected in serum separator tubes, allowed to clot at 25° C. for 20-30 min, and were processed to serum by centrifugation at 5000 rpm for 5 min. The samples were frozen and stored at −80° C.

### Results

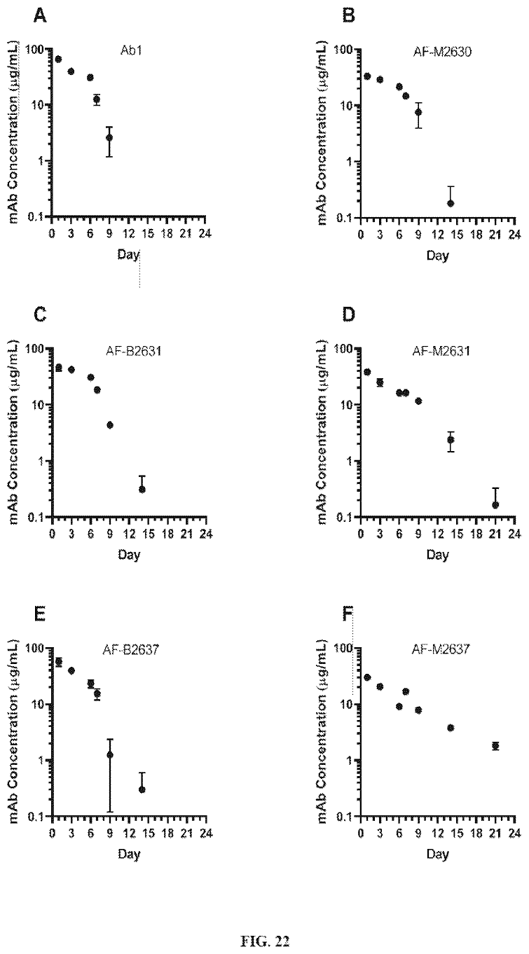

The relationship between ADA and antibody pharmacokinetics was characterized by measuring serum antibody concentrations over the 21 day duration of the experiment. AF-M2637 was present in the serum throughout the duration of the experiment with no acceleration of elimination being observed over 21 days (FIG. 22). These results are consistent with the lack of ADA response observed with this treatment group. The elimination profiles of Ab1, AF-M2630, AF-B2631 and AF-B2637 were similar (FIG. 22). All displayed rapid elimination of serum antibody with an acceleration observed by day 9 (Ab1, AF-B2631 and AF-B2637) or by day 14 (AF-M2630). AF-M2631 was eliminated somewhat slower (FIG. 22D). The timing of the accelerated elimination of Ab1, AF-M2630, AF-B2631 and AF-B2637 all coincided with the rise in ADA observed by day 14 (FIG. 21). In fact, Ab1 was undetectable in the serum by day 14, coinciding with the high titer ADA present in this group at day 14. Ab1, AF-M2630, AF-B2631 and AF-B2637 were all undetectable in serum at day 21 and levels of AF-M2631 were barely detectable. The pharmacokinetics of Ab1 and the variants were consistent with the ADA response. Accelerated terminal elimination was observed with all antibodies that generated an ADA response, but not with AF-M2637 for which no ADA was detected.

### Example 12. Characterization of Binding to Cynomolgus TNFα

Ab1 binds and neutralizes murine TNFα (e.g., SEQ ID NO: 84), cynomolgus monkey TNFα (e.g., SEQ ID NO: 82), and human TNFα (e.g., SEQ ID NO: 78). Additionally, Ab1 is immunogenic in all three species. The initial selection of therapeutic candidates with pH-dependent binding was based on screening against human TNFα for enhanced dissociation following a pH 6.0 wash step, but the candidates also displayed varying degrees of reduced binding affinity to human TNFα at pH 7.4, as summarized in Example 5. The binding activity of these variants against cynomolgus TNFα following wash steps at pH 7.4 and pH 6.0 is unknown.

Clinical candidates will be evaluated for immunogenicity in a cynomolgus model. Interpretation of the results will be facilitated by understanding the impact of the pH-dependent mutations on binding to cynomolgus TNFα. The objective of the studies outlined in this example was to characterize the binding, as well as the pH-dependent dissociation, of Ab1 and variants (pH switch, monovalent and bivalent formats) to cynomolgus TNFα.

### Materials and Methods

### Biotinylation of Cynomolgus TNFα

To biotinylate cynomolgus TNFα (ThermoFisher Scientific) 50 μg was resuspended in 100 μL of water (500 μg/mL final concentration). Immediately prior to use, 1 mg of EZ-Link Sulfo-NHS-LC-Biotin (ThermoFisher Scientific) was resuspended in 180 μL of water and was then diluted 1:4 in water. Next, 1.9 μL of the diluted EZ-Link Sulfo-NHS-LC-Biotin was added to 100 μL of the cynomolgus TNFα to label at a molar ratio of 5:1 (biotin:TNFα) and the reaction proceeded for 30 min at 25° C. at which time it was quenched by the addition of 2 μL of 1M Tris, pH 7.4. The reaction was diluted to 500 μL with PBS and excess unreacted biotin was removed by placing the material on a Zeba 5 mL spin desalting column, 7K MWCO (ThermoFisher Scientific) that had been prepped per manufacturer's directions (4×2.5 mL washes with PBS). Biotinylated cynomolgus TNFα was collected by centrifugation at 1000×g for 2 min and the final collected volume was measured in order to estimate the concentration of biotinylated TNFα (assuming 100% recovery). The final concentration was 80.6 μg/mL or 1.54 mM (assuming cynomolgus TNFα MW=52,500 Da).

### Cynomolgus TNFα Binding ELISA

A 96-well plate was coated with 50 μL of 2 μg/mL goat anti-human kappa antibody (SouthernBiotech) in PBS for 1 h at 25° C. Subsequently, the plate was washed once (all wash steps were performed with PBS, 0.05% Tween 20) and was then blocked with 100 μL of PBS, 1% (w/v) BSA (Fisher Scientific) for 1 h at 25° C. Next, 50 μL of 1 μg/mL test article antibody diluted in PBS, 1% (w/v) BSA was added and incubated for 1 h at 25° C. The plate was washed four times and 50 μL of biotinylated cynomolgus TNFα diluted serially, 3-fold in PBS, 1% (w/v) BSA was incubated for 1 h at 25° C. The plate was washed four times and 50 μL of neutravidin-HRP (ThermoFisher Scientific) diluted 1:2000 in PBS, 1% (w/v) BSA was added for 1 h at 25° C. Alternatively, for prolonged dissociation at pH 6.0 the plate was submerged in 500 mL of PBS, 0.05% Tween 20, pH 6.0 for 1 h at 25° C. prior to the addition of neutravidin-HRP. To ensure thorough washing, buffer was aspirated from the plate every 15 min, followed by re-submersion in 500 mL of wash buffer. The plate was washed four times and was developed using 50 μL/well of Ultra-TMB-ELISA substrate (ThermoFisher Scientific). The reaction was terminated by the addition of 50 μL/well of 2N H₂SO₄ and the absorbance at 450 nm was quantitated using a Spectramax Plus 384 microplate reader (Molecular Devices).

### Results

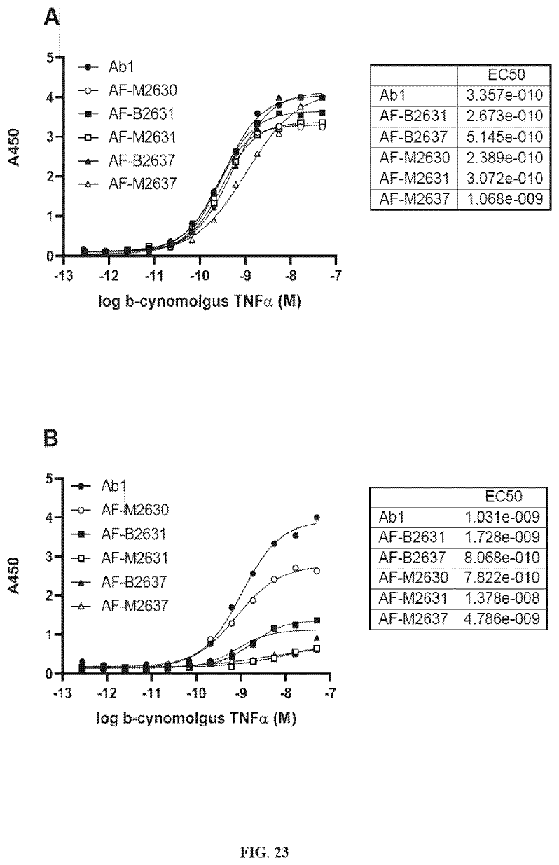

Ab1 and variants were captured on the ELISA plate, and subsequently the binding of biotinylated cynomolgus TNFα was quantitated using neutravidin-HRP conjugate. Ab1 and the variants displayed dose-dependent, saturable binding to cynomolgus TNFα (FIG. 23A) following a pH 7.4 wash step. Ab1 and all variants bound cynomolgus TNFα with high affinity in this ELISA format.

Next, the dissociation of the variants following a prolonged pH 6.0 wash step was evaluated. The pH switch variants displayed significantly less binding than Ab1, regardless of the antibody format/valency (FIG. 23B). Also, in all cases the monovalent variants displayed less binding than the corresponding bivalent construct (FIG. 23B, compare open symbols vs closed symbols). Very little binding was observed with the monovalent pH switch variants AF-M2631 and AF-M2637 (FIG. 23B).

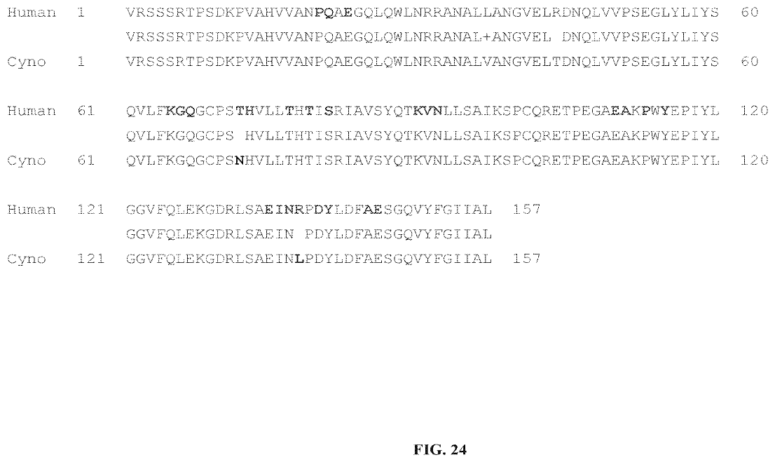

The similar binding of variants to cynomolgus TNFα relative to human TNFα is likely a result of the high sequence identity between the soluble TNFα domain of the species (153/157 residues; 97% identity). The soluble domain of cynomolgus TNFα differs from human TNFα at only two of twenty-six epitope residues identified by Hu et al., 2013 (FIG. 24).

Collectively, these data support the evaluation of the pH switch variants, particularly the monovalent format, in cynomolgus monkey immunogenicity experiments.

### Example 13. Cell Based Potency Assays with Cynomolgus TNFα

Next, the ability of Ab1 and variants to inhibit the binding of soluble cynomolgus TNFα to TNFR was characterized using the HEK-Blue™ cell-based potency assay, as described in Example 8.

### Materials and Methods

Cynomolgus TNFα Dose Response with HEK-Blue™ TNFα Cells

The dose response of cynomolgus TNFα stimulation of HEK-Blue™ cells was performed as described in Example 8, but cells were stimulated with 20 μL of cynomolgus TNFα (MW 52.5 kD), added at 5 ng/ml final concentration and diluted serially, 2-fold.

### HEK-Blue™ TNFα Potency Assay

The experiment was performed as described for human TNFα (Example 8), with one modification. Antibodies were serially diluted 5-fold, beginning at 6 μM, mixed with 50 pM cynomolgus TNFα in a 96-well microplate and incubated 1 h at 25° C. prior to addition to cells.

### Results

To gauge the sensitivity of the cell line to TNFα stimulation, the cells were treated with varying concentrations of cynomolgus TNFα. The cells responded to cynomolgus TNFα in a dose-dependent and saturable manner (FIG. 13). Recombinant cynomolgus TNFα was a potent stimulator of HEK-Blue™ TNFα cells (EC50=3.1 pM). For comparison, the EC50 for stimulation of the cells with human TNFα was 2.5 pM.

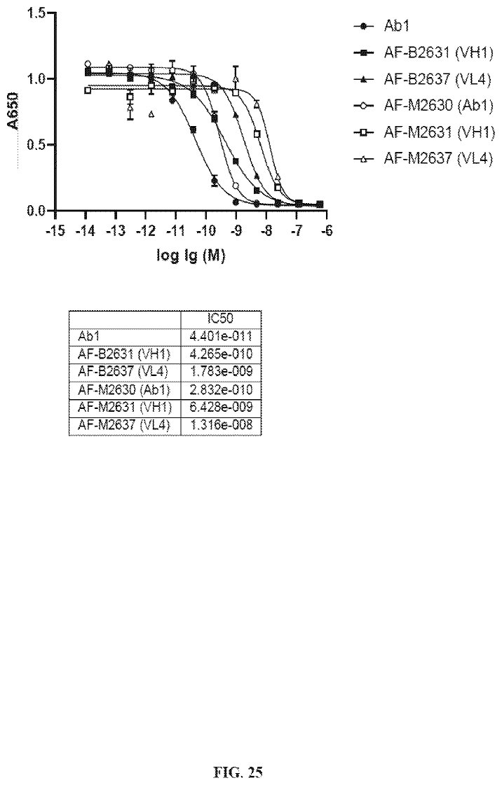

Next, the ability of Ab1 and variants to inhibit stimulation of the cells with cynomolgus TNFα was evaluated. The antibodies were titrated with 5 pM cynomolgus TNFα and the mixture was incubated with the cells for 20 h. Ab1 and the variants displayed a similar 300-fold range of potencies against cynomolgus TNFα (FIG. 25), similar to the range of potencies observed with human TNFα (FIG. 14). Ab1 (closed circles) was the most potent (IC₅₀=44 pM) while the monovalent pH switch variant AF-M2637 (open triangles) was the least potent (IC₅₀=13 nM). The bivalent form of each variant (FIG. 25, closed symbols) was more potent than the corresponding monovalent format (FIG. 25, open symbols) for all three variants. The variants inhibited stimulation of the cells with cynomolgus TNFα in a similar fashion to their inhibition of stimulation of the cells with human TNFα. The similar inhibition of TNFα from both species by Ab1, AF-M2630 and the pH switch variants is consistent with the high degree of sequence identity (97%) between the soluble domains of cynomolgus and human TNFα (FIG. 24). Combined with the binding data, the potency data indicates that cynomolgus monkeys may be a suitable non-human primate model to assess immunogenicity of the variants.

1-53. (canceled)

54. An anti-TNFα antibody or an antigen-binding portion thereof that binds to the same epitope of human TNFα as a reference antibody comprising:
a) a heavy chain (HC) that comprises the amino acid sequence of SEQ ID NO: 2 and a light chain (LC) that comprises the amino acid sequence of SEQ ID NO: 6;
b) an HC that comprises the amino acid sequence of SEQ ID NO: 72 and an LC that comprises the amino acid sequence of SEQ ID NO: 73;
c) an HC that comprises the amino acid sequence of SEQ ID NO: 74 and an LC that comprises the amino acid sequence of SEQ ID NO: 75; or
d) an HC that comprises the amino acid sequence of SEQ ID NO: 76 and an LC that comprises the amino acid sequence of SEQ ID NO: 77;
wherein said anti-TNFα antibody comprises HC and LC at least 90% identical to the HC and LC of the reference antibody, respectively; and
wherein said anti-TNFα antibody or antigen-binding portion i) is monovalent; and
ii) has a binding affinity for TNFα that is lower at pH 6.0 than at pH 7.4.

55. A monovalent anti-TNFα antibody that comprises heavy chain (HC) CDR1-3 and light chain (LC) CDR1-3 comprising:
a) SEQ ID NOs: 55, 56, 57, 58, 59, and 68, respectively;
b) SEQ ID NOs: 55, 56, 63, 58, 59, and 60, respectively;
c) SEQ ID NOs: 55, 56, 62, 58, 59, and 60, respectively;
d) SEQ ID NOs: 55, 56, 57, 58, 59, and 60, respectively;
e) SEQ ID NOs: 55, 56, 61, 58, 59, and 60, respectively;
f) SEQ ID NOs: 55, 56, 57, 64, 59, and 60, respectively;
g) SEQ ID NOs: 55, 56, 57, 58, 59, and 66, respectively;
h) SEQ ID NOs: 55, 56, 57, 58, 59, and 67, respectively;
i) SEQ ID NOs: 55, 56, 57, 65, 59, and 60, respectively;
j) SEQ ID NOs: 55, 56, 57, 58, 59, and 69, respectively;
k) SEQ ID NOs: 55, 56, 61, 64, 59, and 60, respectively;
l) SEQ ID NOs: 55, 56, 61, 58, 59, and 66, respectively;
m) SEQ ID NOs: 55, 56, 61, 58, 59, and 67, respectively;
n) SEQ ID NOs: 55, 56, 61, 58, 59, and 68, respectively;
o) SEQ ID NOs: 55, 56, 61, 65, 59, and 60, respectively;
p) SEQ ID NOs: 55, 56, 61, 58, 59, and 69, respectively;
q) SEQ ID NOs: 55, 56, 62, 58, 59, and 66, respectively;
r) SEQ ID NOs: 55, 56, 62, 58, 59, and 67, respectively;
s) SEQ ID NOs: 55, 56, 62, 58, 59, and 68, respectively;
t) SEQ ID NOs: 55, 56, 62, 65, 59, and 60, respectively;
u) SEQ ID NOs: 55, 56, 63, 58, 59, and 66, respectively;
v) SEQ ID NOs: 55, 56, 63, 58, 59, and 67, respectively;
w) SEQ ID NOs: 55, 56, 63, 58, 59, and 68, respectively;
x) SEQ ID NOs: 55, 56, 63, 65, 59, and 60, respectively;
y) SEQ ID NOs: 55, 56, 63, 58, 59, and 69, respectively; or
z) SEQ ID NOs: 55, 56, 86, 58, 59, and 87, respectively.

56. The monovalent anti-TNFα antibody of claim 55, wherein said antibody comprises a heavy chain variable domain (VH) and a light chain variable domain (VL), wherein said VH and VL comprise:
a) SEQ ID NOs: 4 and 46, respectively;
b) SEQ ID NOs: 28 and 8, respectively;
c) SEQ ID NOs: 22 and 8, respectively;
d) SEQ ID NOs: 4 and 8, respectively;
e) SEQ ID NOs: 16 and 8, respectively;
f) SEQ ID NOs: 4 and 34, respectively;
g) SEQ ID NOs: 4 and 38, respectively;
h) SEQ ID NOs: 4 and 42, respectively;
i) SEQ ID NOs: 4 and 50, respectively;
j) SEQ ID NOs: 4 and 54, respectively;
k) SEQ ID NOs: 16 and 34, respectively;
l) SEQ ID NOs: 16 and 38, respectively;
m) SEQ ID NOs: 16 and 42, respectively;
n) SEQ ID NOs: 16 and 46, respectively;
o) SEQ ID NOs: 16 and 50, respectively;
p) SEQ ID NOs: 16 and 54, respectively;
q) SEQ ID NOs: 22 and 38, respectively;
r) SEQ ID NOs: 22 and 42, respectively;
s) SEQ ID NOs: 22 and 46, respectively;
t) SEQ ID NOs: 22 and 50, respectively;
u) SEQ ID NOs: 28 and 38, respectively;
v) SEQ ID NOs: 28 and 42, respectively;
w) SEQ ID NOs: 28 and 46, respectively;
x) SEQ ID NOs: 28 and 50, respectively;
y) SEQ ID NOs: 28 and 54, respectively; or
z) SEQ ID NOs: 70 and 71, respectively.

57. The monovalent anti-TNFα antibody of claim 56, wherein said antibody comprises
a) a monovalent antigen-binding protein that comprises an HC comprising said VH and an LC comprising said VL; and
b) a truncated HC lacking the variable domain and CH1 domain;
wherein the antigen-binding protein HC and the truncated HC are capable of dimerization.

58. The monovalent anti-TNF antibody of claim 57, wherein
a) the antigen-binding protein HC and the truncated HC comprise knobs-into-holes modifications,
b) the antigen-binding protein HC comprises mutations T366S, L368A, and Y407A in the CH3 domain and the truncated HC comprises the mutation T366W in the CH3 domain, wherein the residues are numbered according to the Eu system,
c) the antigen-binding protein HC comprises the mutation Y349C and the truncated HC comprises the mutation S354C, wherein the residues are numbered according to the Eu system, or
d) the antigen-binding protein HC comprises mutations Y349C, T366S, L368A, and Y407A and the truncated HC comprises the mutations S354C and T366W, wherein the residues are numbered according to the Eu system.

59. The monovalent anti-TNFα antibody of claim 57, wherein the antigen-binding protein HC, the antigen-binding protein LC, and the truncated HC comprise:
a) SEQ ID NOs: 10, 44, and 12, respectively;
b) SEQ ID NOs: 30, 6, and 12, respectively;
c) SEQ ID NOs: 24, 6, and 12, respectively;
d) SEQ ID NOs: 10, 6, and 12, respectively;
e) SEQ ID NOs: 18, 6, and 12, respectively;
f) SEQ ID NOs: 10, 32, and 12, respectively;
g) SEQ ID NOs: 10, 36, and 12, respectively;
h) SEQ ID NOs: 10, 40, and 12, respectively;
i) SEQ ID NOs: 10, 48, and 12, respectively;
j) SEQ ID NOs: 10, 52, and 12, respectively;
k) SEQ ID NOs: 18, 32, and 12, respectively;
l) SEQ ID NOs: 18, 36, and 12, respectively;
m) SEQ ID NOs: 18, 40, and 12, respectively;
n) SEQ ID NOs: 18, 44, and 12, respectively;
o) SEQ ID NOs: 18, 48, and 12, respectively;
p) SEQ ID NOs: 18, 52, and 12, respectively;
q) SEQ ID NOs: 24, 36, and 12, respectively;
r) SEQ ID NOs: 24, 40, and 12, respectively;
s) SEQ ID NOs: 24, 44, and 12, respectively;
t) SEQ ID NOs: 24, 48, and 12, respectively;
u) SEQ ID NOs: 30, 36, and 12, respectively;
v) SEQ ID NOs: 30, 40, and 12, respectively;
w) SEQ ID NOs: 30, 44, and 12, respectively;
x) SEQ ID NOs: 30, 48, and 12, respectively; or
y) SEQ ID NOs: 30, 52, and 12, respectively.

60. The monovalent anti-TNFα antibody of claim 56, wherein said antibody comprises
a) a single-chain variable fragment (scFv) that comprises said VH and said VL, linked to an Fc monomer domain; and
b) a truncated HC lacking the variable domain and CH1 domain;
wherein the Fc monomer domain linked to the scFv, and the truncated HC, are capable of dimerization.

61. The monovalent anti-TNF antibody of claim 60, wherein
a) the Fc monomer linked to the scFv, and the truncated HC, comprise knobs-into-holes modifications;
b) the Fc monomer linked to the scFv comprises mutations T366S, L368A, and Y407A in the CH3 domain and the truncated HC comprises the mutation T366W in the CH3 domain, wherein the residues are numbered according to the Eu system;
c) the Fc monomer linked to the scFv comprises the mutation Y349C and the truncated HC comprises the mutation S354C, wherein the residues are numbered according to the Eu system; or
d) the Fc monomer linked to the scFv comprises mutations Y349C, T366S, L368A, and Y407A and the truncated HC comprises the mutations S354C and T366W, wherein the residues are numbered according to the Eu system.

62. A monovalent anti-TNFα antibody that comprises an HC and an LC comprising:
a) SEQ ID NOs: 2 and 44, respectively;
b) SEQ ID NOs: 26 and 6, respectively;
c) SEQ ID NOs: 20 and 6, respectively;
d) SEQ ID NOs: 14 and 6, respectively;
e) SEQ ID NOs: 2 and 32, respectively;
f) SEQ ID NOs: 2 and 36, respectively;
g) SEQ ID NOs: 2 and 40, respectively;
h) SEQ ID NOs: 2 and 48, respectively;
i) SEQ ID NOs: 2 and 52, respectively;
j) SEQ ID NOs: 14 and 32, respectively;
k) SEQ ID NOs: 14 and 36, respectively;
l) SEQ ID NOs: 14 and 40, respectively;
m) SEQ ID NOs: 14 and 44, respectively;
n) SEQ ID NOs: 14 and 48, respectively;
o) SEQ ID NOs: 14 and 52, respectively;
p) SEQ ID NOs: 20 and 36, respectively;
q) SEQ ID NOs: 20 and 40, respectively;
r) SEQ ID NOs: 20 and 44, respectively;
s) SEQ ID NOs: 20 and 48, respectively;
t) SEQ ID NOs: 26 and 36, respectively;
u) SEQ ID NOs: 26 and 40, respectively;
v) SEQ ID NOs: 26 and 44, respectively;
w) SEQ ID NOs: 26 and 48, respectively; or
x) SEQ ID NOs: 26 and 52, respectively.

63. The monovalent anti-TNFα antibody or antigen-binding portion of claim 54,
wherein said antibody or antigen-binding portion
a) has a binding affinity for human TNFα that is lower at pH 6.0 than at pH 7.4;
b) binds to human TNFα with a K_{D} of 50 nM or less at pH 7.4 and has a kais of 2e-004 s⁻¹ or greater at pH 6.0;
c) inhibits TNFα stimulation of monocytes;
d) does not form large immune complexes;
e) binds membrane-associated TNFα;
f) is less immunogenic in vivo than an antibody comprising an HC that comprises SEQ ID NO: 2 and an LC that comprises SEQ ID NO: 6;
g) has a longer half-life in vivo than an antibody comprising an HC that comprises SEQ ID NO: 2 and an LC that comprises SEQ ID NO: 6; or
h) any combination of a)-g).

64. A bispecific binding molecule having the binding domain of an anti-TNFα antibody of claim 54 and the binding domain of a second, distinct antibody.

65. An immunoconjugate comprising an anti-TNFα antibody or antigen-binding portion of claim 54 linked to a therapeutic agent.

66. Isolated nucleic acid molecule(s) comprising nucleotide sequences that encode the heavy and light chains of the anti-TNFα antibody or antigen-binding portion of claim 54.

67. Vector(s) comprising the isolated nucleic acid molecule(s) of claim 66, wherein the vector(s) further comprise expression control sequence(s) linked operatively to the isolated nucleic acid molecule(s).

68. A host cell comprising a nucleotide sequence that encodes the heavy chain sequence(s), and a nucleotide sequence that encodes the light chain sequence, of the anti-TNFα antibody or antigen-binding portion of claim 54.

69. A method for producing an anti-TNFα antibody or an antigen-binding portion thereof, comprising providing the host cell of claim 68, culturing said host cell under conditions suitable for expression of the antibody or antigen-binding portion, and isolating the resulting antibody or antigen-binding portion.

70. A pharmaceutical composition comprising the anti-TNFα antibody or antigen-binding portion of claim 54 and a pharmaceutically acceptable excipient.

71. A method for treating an autoimmune or inflammatory condition in a patient in need thereof, comprising administering to said patient a therapeutically effective amount of the anti-TNFα antibody or antigen-binding portion of claim 54.

72. The method of claim 71, wherein the autoimmune or inflammatory condition is rheumatoid arthritis, psoriatic arthritis, plaque psoriasis, ankylosing spondylitis, axial spondyloarthritis, Crohn's disease, ulcerative colitis, hidradenitis suppurativa, polyarticular juvenile idiopathic arthritis, panuveitis, or Alzheimer's disease.

73. The method of claim 71, wherein the patient is treated with an additional therapeutic agent, wherein the additional therapeutic agent is methotrexate.
